SEQUENCE LISTING (8 sequences)

SEQ ID NO: 1 - DNA, 62 bp, synthetic construct
  source 1..62: mol_type = other DNA
  modified_base 51..52: mod_base = OTHER; note = Thymidine-succinyl hexamide CED phosphoramidite
agacaatcaaccatttggggtggacagccttgacctctagacttcggcattttttttttttt

SEQ ID NO: 2 - DNA, 112 bp, synthetic construct
  source 1..112: mol_type = other DNA
  modified_base 101..102: mod_base = OTHER; note = Thymidine-succinyl hexamide CED phosphoramidite
cgggatccttatcgtcatcgtcgtacagatcccgacccatttgctgtccaccagtcatgctagccataccatgatgatgatgatgatgagaaccccgcattttttttttttt

SEQ ID NO: 3 - DNA, 19 bp, synthetic construct
  source 1..19: mol_type = other DNA
atgcggggttctcatcatc

SEQ ID NO: 4 - DNA, 20 bp, synthetic construct
  source 1..20: mol_type = other DNA
cgggatccttatcgtcatcg

SEQ ID NO: 5 - DNA, 20 bp, synthetic construct
  source 1..20: mol_type = other DNA
aatgatacggcgaccaccga

SEQ ID NO: 6 - DNA, 24 bp, synthetic construct
  source 1..24: mol_type = other DNA
caagcagaagacggcatacgagat

SEQ ID NO: 7 - DNA, 21 bp, synthetic construct
  source 1..21: mol_type = other DNA
atcatccgtcggaccgcgagc

SEQ ID NO: 8 - DNA, 21 bp, synthetic construct
  source 1..21: mol_type = other DNA
  misc_feature 1..2: note = DNA
  misc_feature 3: note = RNA
  misc_feature 4..5: note = DNA
  misc_feature 6: note = RNA
  misc_feature 7..9: note = DNA
  misc_feature 10: note = RNA
  misc_feature 11..13: note = DNA
  misc_feature 14: note = RNA
  misc_feature 15..16: note = DNA
  misc_feature 17: note = RNA
  misc_feature 18..20: note = DNA
  misc_feature 21: note = RNA
attattcgttggatcgtgagt

(19) United States
(12) Patent Application Publication
(10) Pub. No.: US 2026/0250758 A1
(43) Pub. Date: Aug. 27, 2026

(54) LIBRARIES FOR METHYLATION ANALYSIS

(71) Applicant: Twist Bioscience Corporation, South San Francisco, CA (US)

(72) Inventors: Bryan N. HÖGLUND, Carlsbad, CA (US); Holly CORBITT, Portland, OR (US); Leonardo ARBIZA, San Francisco, CA (US); Kristin D. BUTCHER, San Francisco, CA (US); Brenton I.M. GRAHAM, San Francisco, CA (US); Ramsey Ibrahim ZEITOUN, San Francisco, CA (US)

(21) Appl. No.: 18/852,993

(22) PCT Filed: Mar. 31, 2023
(86) PCT No.: PCT/US2023/017191

§ 371 Date: Sep. 30, 2024

Related U.S. Application Data
(60) Provisional application No. 63/326,647, filed on Apr. 1, 2022.

(51) Int. Cl. C12Q 1/6869; C12N 15/10; C12Q 1/34; C12Q 1/6806; C12Q 1/6827

(52) U.S. Cl. CPC C12Q 1/6869; C12N 15/1093; C12Q 1/34; C12Q 1/6806; C12Q 1/6827; C12Q 2600/156; C12Q 2600/166; G01N 2333/978

(57) ABSTRACT

Provided herein are compositions and methods for identifying post-transcriptional nucleic acid modifications. Further provided herein are synthetic blocking libraries. Further provided herein are methods for designing synthetic blocking libraries, and application towards methylome analysis.

### CROSS-REFERENCE

This application claims the benefit of U.S. provisional patent application No. 63/326,647 filed on Apr. 1, 2022 which is incorporated by reference in its entirety.

### BACKGROUND

Nucleic acid analysis with high fidelity and low cost has a central role in biotechnology and medicine, and in basic biomedical research. While various methods are known for analyzing complex nucleic acid samples via hybridization-based processes, these techniques often suffer from scalability, automation, speed, accuracy, and cost.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are methods for detection of methylated nucleic acids, comprising: (a) contacting a probe library comprising a plurality of polynucleotide probes with a plurality of genomic fragments comprising methylated nucleic acids; (b) enriching at least one genomic fragment that binds to at least one polynucleotide probe in the probe library to generate at least one enriched target polynucleotide; and (c) sequencing the at least one enriched target polynucleotide. Further provided herein are methods wherein the method further comprises deamination of cytosine in the plurality of genomic fragments prior to step (a). Further provided herein are methods wherein deamination comprises treatment with bisulfite or one or more enzymes. Further provided herein are methods wherein the enzyme is APOBEC (“apolipoprotein B mRNA editing enzyme, catalytic polypeptide-like”). Further provided herein are methods wherein the one or more enzymes are APOBEC and TET2. Further provided herein are methods wherein the probe library is configured to hybridize to at least one genomic fragment comprising a CpG island. Further provided herein are methods wherein the probe library is configured to hybridize to at least one genomic fragment comprising 5-methylcytosine or 5-hydroxymethlycytosine. Further provided herein are methods wherein the probe library comprises at least 5000 polynucleotide probes. Further provided herein are methods wherein the probe library comprises at least 1 million polynucleotide probes. Further provided herein are methods wherein the probe library comprises at least 10 million polynucleotide probes. Further provided herein are methods wherein the polynucleotide probes are 80-250 bases in length. Further provided herein are methods wherein the polynucleotide probes are double stranded. Further provided herein are methods wherein the polynucleotide probes are configured to bind to both sense and antisense regions of a target polynucleotide. Further provided herein are methods wherein the polynucleotide probes are configured to bind to both treated and untreated genomic fragments. Further provided herein are methods wherein the library is present in at least 5 fold molar excess over the plurality of genomic fragments. Further provided herein are methods wherein the polynucleotide probes comprise at least one detectable label. Further provided herein are methods wherein the polynucleotide probes collectively comprise at least 1 million bases. Further provided herein are methods wherein the polynucleotide probes collectively comprise at least 10 million bases. Further provided herein are methods wherein the polynucleotide probes collectively comprise at least 100 million bases. Further provided herein are methods wherein the polynucleotide probes collectively target one or more of CpG islands, CpG shores, CpG shelves and CpG open seas. Further provided herein are methods wherein the polynucleotide probes collectively target at least 3 million CpG sites. Further provided herein are methods wherein the polynucleotide probes collectively target at least 6 million CpG sites. Further provided herein are methods wherein the polynucleotide probes collectively target at least 80% of all CpG sites in the human genome. Further provided herein are methods wherein the polynucleotide probes collectively target at least 25 million bases comprising one or more regulatory features involved in methylation. Further provided herein are methods wherein the regulatory features comprise CTCF binding sites, enhancers, open chromatin regions, promoters or transcription factor binding sites. Further provided herein are methods wherein the polynucleotide probes are not attached to a solid support during enriching. Further provided herein are methods wherein binding comprises hybridization. Further provided herein are methods wherein at least some of the polynucleotide probes are configured to bind to BRCA1 proximal enhancer region. Further provided herein are methods wherein the polynucleotide probes are configured to bind to CpG sites associated with at least one cancer. Further provided herein are methods wherein the polynucleotide probes are configured to bind to CpG sites associated with at least 20 cancers. Further provided herein are methods wherein sequencing comprises sequencing by synthesis, nanopore sequencing, or SMRT sequencing. Further provided herein are methods wherein at least 5 million CpG sites are sequenced with at least 10× coverage. Further provided herein are methods wherein the method further comprises contacting the library with salmon sperm in step (a). Further provided herein are methods wherein contacting occurs for no more than 4 hours. Further provided herein are methods wherein contacting occurs at a temperature of 60-70 degrees C. Further provided herein are methods wherein at least some of genomic fragments comprise at least one polynucleotide adapter. Further provided herein are methods wherein the at least one polynucleotide adapter comprises at least one index sequence. Further provided herein are methods wherein the at least one index sequence is 8-16 bases in length. Further provided herein are methods wherein the method further comprises contacting the library with one or more universal blockers in step (a). Further provided herein are methods wherein the plurality of genomic fragments comprise cell-free DNA. Further provided herein are methods wherein the plurality of genomic fragments comprise ctDNA. Further provided herein are methods wherein the plurality of genomic fragments are 100-200 bases in length. Further provided herein are methods wherein the plurality of genomic fragments each comprise at least one adapter. Further provided herein are methods wherein the adapter comprises a unique molecular identifier (UMI). Further provided herein are methods wherein the adapter comprises at least one methylated base. Further provided herein are methods wherein all cytosines in the adapter are methylated. Provided herein are methylome detection systems comprising: a probe library provided herein; one or more enzymes configured to distinguish methylated from unmethylated bases; a plurality of sample nucleic acids; and optionally a methylation control. Further provided herein are systems wherein the methylation control comprises a plurality of polynucleotides comprising at least eight different amounts of methylation. Further provided herein are systems wherein the methylation control comprises at least 24 different sequences. Further provided herein are systems wherein the methylation control comprises at least 24 different sequences having no homology to sequences in the Refseq database. Further provided herein are systems wherein the methylation control comprises polynucleotides having an average length of 150-175 bases. Further provided herein are systems wherein the methylation control comprises polynucleotides having an average length of about 167 bases. Further provided herein are systems wherein the sample nucleic acids comprise cell-free DNA. Further provided herein are systems wherein the plurality of genomic fragments comprise ctDNA.

### BRIEF DESCRIPTION OF THE DRAWINGS

FIG. 1A depicts a workflow for targeted methylome analysis. Methylation sequencing involves enzymatic or chemical methods of converting unmethylated cytosines to uracil through deamination, while leaving methylated cytosines intact. During amplification, uracil is paired with adenine on the complementary strand, leading to the inclusion of thymine in the original position of the unmethylated cytosine. The end product is asymmetric, yielding two different double stranded DNA molecules after conversion (top row); the same process for methylated DNA leads to yet additional sets of sequences (bottom row).

FIG. 1B depicts a workflow for enzymatic conversion of unmethylated cytosines to identify sites of methyl-cytosine (5mC) and hydroxymethyl-cytosine (5hmC).

FIG. 2A depicts a conversion rate comparison for bisulfite (left) and enzymatic (right) conversion. Conversion rates, measured as the percentage of cytosines converted to thymine in non-CpG sites, were >99.5% for both library conversion methods. The y-axis is labeled 90-100% at 2% intervals.

FIG. 2B depicts coverage by target GC content for bisulfite and enzymatic conversion. Both library conversion approaches are compatible with the blocking libraries described herein, although improved hybrid selection metrics are observed for libraries prepared with the enzymatic conversion approach. High GC target regions are associated with lower coverage when using the bisulfite conversion method (left), while a less severe bias is observed when using the enzymatic conversion method (right). The y-axis is labeled as “target read counts” from 0-300 at 50 count intervals. The x-axis is labeled GC content of target (%) from 20-100 at 20% intervals.

FIG. 2C depicts a quality control step after using the EM-seq conversion method for library preparation. The average peak length is approximately 375 bp. The y-axis is labeled 0-250 at 50 fluorescent unit intervals; the x-axis is labeled at 50, 300, 500, 1000, and 10380 base pair intervals.

FIG. 2D depicts a comparison of conversion rates (percent) for enzymatic (left) and bisulfite (right) methods. The y-axis is labeled conversion rate from 99.5-100.0 at 0.1% intervals.

FIG. 2E depicts a comparison of library yields (ng/μL) for enzymatic and bisulfite methods. The x-axis is labeled (left to right, with numbers representing library concentration in ng/microliter): bisulfite control (8.8); bisulfite-1 (51.7); bisulfite-2 (101); enzymatic (112). The y-axis is labeled as the concentration of the DNA library (ng/microliter) from 0-120 at 20 ng/microliter intervals.

FIG. 2F depicts a comparison of library product lengths (bp) for bisulfite methods. The x-axis is labeled (left to right, with numbers representing average sizes (base pairs)): bisulfite control (287); bisulfite-1 (338); bisulfite-2 (346). The y-axis is labeled as the average size of the DNA library (base pairs) from 0-600 at 100 base pair intervals.

FIG. 2G depicts a comparison of library product lengths (bp) for an enzymatic method. The x-axis is labeled bisulfite control (548 average bp size). The y-axis is labeled as the average size of the DNA library (base pairs) from 0-600 at 100 base pair intervals.

FIG. 2H depicts a plot of the percentage methylation of cytosines in enzymatic conversion method vs. percentage methylation of cytosines in bisulfite conversion method (left), with r2=0.96. The number of CpGs detected for the bisulfite method (left two bars) and enzymatic method (right two bars) are shown on the right. 15% more CpGs were detected with the enzymatic method. The left graph y-axis is labeled percentage methylation of cytosines in enzymatic conversion method, and the x-axis is labeled percentage methylation of cytosines in bisulfite conversion method, from 0.00 to 1.00 at 0.25 intervals. The right graph y-axis is labeled the number of detected CpGs from 0 to 1.5×10⁷ at 0.5×10 intervals, and the x-axis is labeled replicates of control cfDNA (the two left bars are bisulfite method, and the two right bars are enzymatic method).

FIG. 2I depicts percent off bait sequencing metrics for enzymatic and bisulfite methods. The y-axis is labeled Pct Off Bait (0-100% at 20% intervals), the x-axis is labeled library kit/expected methylation fraction from 0-1 at 0.25 unit intervals. Circular data points represent data points generated from 4 h fast hybridization, squares represent data points generated from a standard 16 hour hybridization.

FIG. 2J depicts fold-80 base penalty sequencing metrics for enzymatic and bisulfite methods. The y-axis is labeled fold-80 base penalty (1.2-2.2 at 0.2 intervals), the x-axis is labeled library kit/expected methylation fraction from 0-1 at 0.25 unit intervals. Circular data points represent data points generated from 4 h fast hybridization, squares represent data points generated from a standard 16 hour hybridization.

FIG. 3A depicts a reduction in off-target for 1.28 Mb (left pair of bars) and 1.52 Mb (right pair of bars) custom methylation panels generated through two design pipelines. The y-axis is labeled Off Target (%) from 0-60 at 10% intervals. The left bar in each set used design 1, the right bar in each set used design 2.

FIG. 3B depicts improved picard metrics with panels (1.28 Mb and 1.52 Mb) designed against both the plus and minus strands. The figure to the right show the Fold-80 (uniformity, y-axis labeled 1.0-2.4 at 0.2 unit intervals) and Hs Library Size (number of unique molecules, y-axis labeled 0.0-2.5 at 0.5 unit intervals) for two panels designed against the plus stand only (left bars) or the plus and minus strands (Plus/Minus shown as the right bars).

FIGS. 4A-4D depict Picard Metrics using a synthetic blocking library of design 2 at various fast wash buffer 1 temperatures. Hybrid capture was performed using different sized custom methylation panels and 200 ng of library (NA12878; Coriell) and a 4-hour hybridization time with variation in the fast wash buffer 1 temperature (left to right: room temperature, 55, 60, 63, 66, 70 degrees). Custom methylation panels were designed using no stringency filters to best determine how off-target is impacted. FIG. 4A depicts the percentage of off-target molecules (y-axis is labeled 0-100 at 20 unit intervals, x-axis panels left to right: 0.04 Mb, 1.28 Mb, 3.00 Mb).

FIG. 4B depicts uniformity represented by the Fold-80 metric (y-axis is labeled 1.0-3.5 at 0.5 unit intervals, x-axis panels left to right: 0.04 Mb, 1.28 Mb, 3.00 Mb) and shows decreases as the fast wash buffer 1 temperature (left to right: room temperature (RT), 55, 60, 63, 66, 70 degrees) goes up, but starts to increase at temperatures higher than ~66° C. FIG. 4C depicts coverage at 30× (y-axis is labeled 0-100 at 20 unit intervals, x-axis panels left to right: 0.04 Mb, 1.28 Mb, 3.00 Mb) initially increases as the fast wash buffer 1 temperature (left to right: room temperature, 55, 60, 63, 66, 70 degrees) increases, but starts to decrease as the temperature increases over 66° C. FIG. 4D depicts various sequencing metrics as a function of wash buffer temperature (y-axis labeled qualitative values, x-axis labeled wash buffer 1 temperature: RT, 55, 60, 63, 66, 70 degrees C.).

FIG. 5 depicts performance of a synthetic blocking library of design 2 with two methylome targeting enrichment panels. Use of such libraries while decreasing hybridization time leads to a rescue of the off-target metric. Hybrid captures were performed using a 1.28 Mb and 1.52 Mb custom methylation panel and 200 ng of library (NA12878, Coriell) with a fast wash buffer 1 temperature of 63° C. for either 2 hr or 4 hr hybridization times. Custom methylation panels were designed using no stringency filter to best determine how off-target is impacted. Left bars in each set represent reaction with no synthetic blocking library, while the right bars in each set represent reactions 40 ug using design 2. Left graph: 1.28 Mb panel (y-axis labeled off target (%) 10-60 at 10% intervals); right graph: 1.52 Mb panel (y-axis labeled off target (%) 10-60 at 10% intervals).

FIG. 6 depicts off-target metrics using a 2-hour hybridization time with the fast hybridization system and three custom methylation panels ranging in different sizes, represented by color. Fast wash buffer 1 temperature is at 63 degrees C. The y-axis is labeled percent off bait from 0-90% at 10% intervals. The x-axis shows variable amounts of blocking library design 2 added to the system (left to right: 0, 5, 25, 50, 100 micrograms). Genomic DNA used in this figure includes NA12878 (Coriell). Panels are labeled as anchorV1 (open circles); Massie (low stringency, *); 3 Mb (+).

FIG. 7 depicts off-target metrics using a 16-hour hybridization time with the standard hybridization system and three custom panels ranging in different sizes, represented by color. Wash buffer 1 temperature is at 63 degrees C. The y-axis is labeled percent off bait from 0 to 60% at 10% intervals. The x-axis shows variable amounts of blocking library design 2 added to the system. Thermo Cot-1 mass input is labeled as circles (0 micrograms), diamonds (5 micrograms), or X's (40 micrograms). Genomic DNA used in this figure includes NA12878 (Coriell), EpiScope® Methylated HCT116 gDNA (Takara®), and EpiScope® Unmethylated HCT116 DKO gDNA (Takara®). The left half of the graph depicts data using the NEBNext protocol with NA12878 and the x-axis is labeled (left to right) 0, 5, 25, 40, 50, 60, 80, and 100 micrograms. Panels are labeled as anchorV1 (open circles, diamonds or X's); Massie (low stringency, *); 50 Mb (+). The right half of the graph is labeled TotalPure and split into four different conditions (NA12878; 012 methyl/unmethylated; 0.5 methyl/unmethylated-blend; and 0.8 methyl/unmethylated blend. The left data points in each set of conditions represents 0 micrograms blocking design 2 added, and the right data points in each set of conditions represents 40 micrograms blocking design 2 added.

FIG. 8 depicts off-target metrics using two different hybridization times in the fast hybridization system. Three custom methylation panels are used with varying amounts of blocking library design 2 (“methylation enhancer”). The left three sets of conditions were performed with the fast hybridization buffer for 2 h, and the right three sets of conditions were performed with the fast hybridization buffer for 4 hr. The y-axis is labeled 0-50% at 5% intervals; the x-axis is labeled (left to right): 1.23 Mb (Genecast-V3-2), 1.28 Mb (Massie), 1.52 Mb (AnchorV1) for each set of conditions. Methylation enhancer input is labeled as circles (0 micrograms), diamonds (40 micrograms), or X's (100 micrograms).

FIG. 9A depicts graphs of off-Target (%, left, y-axis labeled 0-25% at 5 unit intervals)) and fold-80 base penalty (right, y-axis labeled 1.0-2.0 at 0.2 unit intervals) obtained in the presence or absence of methylation enhancer (library 2) for 1.0 Mb and 1.5 Mb libraries. The left bar in each set represents 0 microliter methylation enhancer volume input and the right bar in each set represents 2 microliter methylation enhancer volume input.

FIG. 9B depicts graphs of 30× coverage (%, left, y-axis labeled 0-100 at 20% intervals) and duplication rate (%, right, y-axis labeled 0-10 at 2% intervals) obtained in the presence or absence of methylation enhancer (library 2) for 1.0 Mb and 1.5 Mb libraries. The left bar in each set represents 0 microliter methylation enhancer volume input and the right bar in each set represents 2 microliter methylation enhancer volume input.

FIG. 9C depicts graphs of off-Target (%, left, y-axis labeled 0-35 at 5% intervals), fold-80 base penalty (middle, y-axis labeled 1.0-2.0 at 0.2 unit intervals, and mean target coverage (x reads, y-axis labeled 60-130 at 10 unit intervals) obtained in the presence or absence of methylation enhancer for three different library sizes (1 Mb, 1.5 Mb, and 50 Mb). The left bar in each pair represents 0 microliters of methylation enhancer added, the right bar in each set represents 2 microliters of methylation enhancer added.

FIG. 9D depicts a graph of Off-target percent for various panel sizes and different amounts of methylation enhancer mass input (micrograms). The y-axis is labeled 0-70 at 10% intervals, and the x-axis is labeled (left to right) with panel sizes 1 Mb, 1.5 Mb, 3 Mb, and 50 Mb. The bars for each panel correspond to 0, 5, 25, 50, and 100 micrograms of methylation enhancer mass input.

FIG. 10 depicts detection of DMRs (Differentially Methylated Regions). DMRs were captured, ranging from 0 to 100% methylation, with minimal or no impact on sequencing metrics, including 30× coverage and uniformity (fold-80 base penalty). Left to right: 30× coverage (%, y-axis labeled 0-100 at 20 unit intervals); fold-80 base penalty (y-axis labeled 1-2.25 at 0.25 unit intervals); percent off bait (%, y-axis labeled 1-60 at 10 unit intervals), duplicate rate (%, y-axis labeled 1-5 at 1 unit intervals). The bars on the x-axis (left to right) are labeled <5%, 25%, 50%, 75%, and 100%.

FIG. 11 depicts a graph of methylation detection in the CCND2 locus. Windows show 100, 75, 50, 25, and 0% methylation (top to bottom). The lower window shows gene, targets, and CpG islands.

FIG. 12A depicts a size of a target region within a custom panel and its relationship to Picard Metrics for custom panels covering target sizes of 0.5 Mb, 3 Mb, and 50 Mb. From left to right: off-target (%, y-axis labeled 0-30 at 5 unit intervals); fold-80 base penalty (y-axis labeled 1.0-2.0 at 0.2 unit intervals); 30× coverage (%, y-axis labeled 0-100 at 20 unit intervals), duplicate rate (%, y-axis labeled 0-14 at 2 unit intervals). The bars on the x-axis (left to right) are labeled 0.5, 3, and 50 Mb panel sizes.

FIG. 12B depicts coverage by Target GC Content for Hypo- (top graph) and Hypermethylated (bottom graph) gDNA Libraries Prepared with Enzymatic and Bisulfite Conversion Techniques. Enzymatic (teal) and bisulfite (grey) conversion library preparation methods were used to make libraries from hypo- and hypermethylated human control human cell lines. Capture was performed using a custom 1.5 Mb panel and a single-plex reaction. The Y-axis is labeled mean target coverage from 0-200 at 100 unit intervals; the x-axis is labeled GC content of target (%) from 30-80 at 10 unit intervals.

FIG. 13 depicts a schematic for fragmenting a sample, end repair, A-tailing, ligating universal adapters, and adding barcodes to the adapters via PCR amplification to generate a sequencing library. Additional steps optionally include enrichment, additional rounds of amplification, and/or sequencing (not shown).

FIG. 14 depicts an image of a plate having 256 clusters, each cluster having 121 loci with polynucleotides extending therefrom.

FIG. 15A depicts a plot of polynucleotide representation (polynucleotide frequency versus abundance, as measured absorbance) across a plate from synthesis of 29,040 unique polynucleotides from 240 clusters, each cluster having 121 polynucleotides.

FIG. 15B depicts a plot of measurement of polynucleotide frequency versus abundance absorbance (as measured absorbance) across each individual cluster, with control clusters identified by a box.

FIG. 16 illustrates a computer system.

FIG. 17 is a block diagram illustrating an architecture of a computer system.

FIG. 18 is a diagram demonstrating a network configured to incorporate a plurality of computer systems, a plurality of cell phones and personal data assistants, and Network Attached Storage (NAS).

FIG. 19 is a block diagram of a multiprocessor computer system using a shared virtual address memory space.

FIG. 20A depicts a pie graph showing targets of a 123 Mb methylome probe design covering 3.97 million CpG sites in the human genome. The pie graph is labeled with 8% CpG shelves, 21% CpG shores, 57% CpG open seas (interCGI), and 15% CpG islands (CGIs). The graphic of a genetic locus under the pi graph is labeled open sea (interCGI), CpG shelf, CpG shore, CpG island, CpG shore, CpG shelf, and open sea (interCGI).

FIG. 20B depicts a graph of different target features in a 123 Mb methylome probe design, showing the total number of base pairs covered in the methylome for each feature. Targets were allowed to be in more than one category to account for different transcripts. Bars are labeled (left to right): enhancers fantom (8,459,549); genes promoters (54,385,728); genes 1to5 kb (49,252,541); genes introns (90,059,139); genes exons (51,290,394); genes SUTRs (21,743,694); genes 3UTRs (10,810,132).

FIG. 21A depicts NGS performance metrics of a 123 Mb methylome probe design including aligned coverage depth (upper left), mean bait coverage (upper right), percent target bases at 30× (lower left), and zero coverage targets percent (lower right). Upper left (aligned_cov_depth(x), y-axis labeled 50-250 at 50 unit intervals); upper right (mean_bait_coverage(x), y-axis is labeled 0-150 at 50 interval units); lower left (PCT_target_bases_30×, y-axis labeled 0.0-1.0 at 0.2 unit intervals), and lower right (zero_cvg_targets_pct, y-axis labeled 0.000-0.010). The x-axis in each graph is labeled (left right): 100×, 150×, 200×, and 250×.

FIG. 21B depicts NGS performance metrics of a 123 Mb methylome probe design including percent off bait (upper left), fold80 base penalty (upper right), percent duplicates removed (lower left), and the unique number of molecules in the library (lower right).

FIG. 21C depicts percent target bases vs. depth of coverage for a 123 Mb methylome probe design. The y-axis is labeled PCT_TARGET_BASES from 0-1.0 at 0.2 unit intervals; the x-axis is labeled depth of coverage at 1×, 10×, 20×, 30×, 40×, 50×, and 100×.

FIG. 21D depicts NGS sequencing metrics for single plex (left bars) and 8-plex samples (right bars). Upper left: Off-target (%), (y-axis is labeled from 0-25 at 5 unit intervals; x-axis is labeled as 50×, 100×, 150×, and 250×); upper right: Fold-80 base penalty (y-axis is labeled from 1.0-1.8 at 0.2 unit intervals; x-axis is labeled as 50×, 100×, 150×, and 250×); lower left: 30× coverage (%), (y-axis is labeled from 0-100 at 20 unit intervals; x-axis is labeled as 50×, 100×, 150×, and 250×); lower right: Zero Coverage Targets (%), (y-axis labeled from 0.00-1.00 at 0.25 unit intervals; x-axis labeled as 50×, 100×, 150×, and 250×).

FIG. 21E depicts NGS sequencing metrics for single plex (left bars in each set) and 8-plex samples (right bars in each set). Upper left: All Dupes (%), (y-axis is labeled from 0-12 at 3 unit intervals; x-axis is labeled as 50×, 100×, 150×, and 250×); upper right: HS Library Size (y-axis is labeled from 0.00-1 at 0.25 unit intervals; x-axis is labeled as 50×, 100×, 150×, and 250×); lower left: AT Dropout (%), (y-axis is labeled from 0-15 at 5 unit intervals; x-axis is labeled as 50×, 100×, 150×, and 250×); lower right: GC Dropout (%), (y-axis labeled from 0-3 at 1 unit intervals; x-axis labeled as 50×, 100×, 150×, and 250×).

FIG. 22 depicts NGS sequencing metrics (left to right: fold enrichment, uniformity, on-target, and off-bait) for a targeted methylation panel described herein (1; left bar in each graph) vs. a commercially available comparator kit (2; right bar in each graph). Fold Enrichment (y-axis is labeled 0-1600 at 400 unit intervals); uniformity (0-5 at 1 unit intervals); On-target (40-65 at 5 unit intervals); Off-bait (0-0.5 at 0.1 unit intervals).

FIG. 23A depicts methylation vs. regions of interest for a methylation panel targeting cfDNA of tumor or control samples. The y-axis is labeled methylation from 0.00 to 1.00 at 0.25 unit intervals. The x-axis is labeled regions of interest.

FIG. 23B depicts methylation vs. regions of interest for a methylation panel targeting cfDNA of tumor or control samples. The y-axis is labeled methylation from 0.00 to 1.00 at 0.25 unit intervals. The x-axis is labeled regions of interest.

FIG. 24 depicts graphs of sequencing metrics obtained for wheat using a synthetic blocking panel. Left graph: Off-target (%), y-axis labeled 0-40 at 5 unit intervals, x-axis labeled Wheat enhancer mass input (0, 40, 120 micrograms); middle graph: Off-target (%), y-axis labeled 0-40 at 10 unit intervals, x-axis labeled total blocker input (5, 40 micrograms; left bar in each set was thermo cot-1, right bar in each set was a synthetic wheat-specific library described herein); right graph: 20× coverage (%), y-axis labeled 40-70 at 10 unit intervals, x-axis is labeled total blocker input (5, 40 micrograms; left bar in each set was thermo cot-1, right bar in each set was a synthetic wheat-specific library described herein). The dashed line indicates a no cot blocker control (mean, n=2).

FIG. 25A depicts a plot of aligned coverage vs. four different sequencing coverage depths. The y-axis is labeled 0-200 at 50 unit intervals. The x-axis is labeled (left to right) 100×, 150×, 200×, and 250×. Values for each sequencing depth were 91.1 (100×), 136.7 (150×), 182.2 (200×), 227.8 (250×).

FIG. 25B depicts a plot of mean target coverage vs. four different sequencing coverage depths. The y-axis is labeled 0-100 percent at 10 unit intervals. The x-axis is labeled (left to right) 100×, 150×, 200×, and 250×. Values for each sequencing depth were 39.4 (100×), 57.16 (150×), 74.27 (200×), 90.52 (250×).

FIG. 25C depicts plots of percent target bases at 30× (top graph) and 50× (bottom graph) vs. four different sequencing coverage depths. The top graph y-axis is labeled 60-100 percent at 10 unit intervals. The top graph x-axis is labeled (left to right) 100×, 150×, 200×, and 250×. Top graph values for each sequencing depth were 71.58 (100×), 90.13 (150×), 95.43 (200×), 97.30 (250×). The bottom graph y-axis is labeled 0-100 percent at 50 unit intervals. The bottom graph x-axis is labeled (left to right) 100×, 150×, 200×, and 250×. Bottom graph values for each sequencing depth were 24.06 (100×), 61.16 (150×), 80.32 (200×), 89.01 (250×).

FIG. 25D depicts plots for NovaSeq flow cell runs.

FIG. 26A depicts a plot of CpG methylation percent vs. sequencing coverage depth. The y-axis is labeled 0-100 percent at 10 unit intervals. The x-axis is labeled (left to right) 100×, 150×, 200×, and 250×.

FIG. 26B depicts a plot of CpG methylation percent vs. sequencing coverage depth for sample cg20185525. The y-axis is labeled 0-100 percent at 10 unit intervals. The x-axis is labeled (left to right) 100×, 150×, 200×, and 250×.

FIG. 26C depicts a plot of CpG methylation percent vs. sequencing coverage depth for sample cg05769317. The y-axis is labeled 0-100 percent at 10 unit intervals. The x-axis is labeled (left to right) 100×, 150×, 200×, and 250×.

FIG. 27 depicts a plot of the methylation fraction for the BRCA1 proximal enhancer region for 100, 150, 200, and 250× coverage. The y-axis is labeled percent methylation from 50-100 percent at 10 unit intervals. The x-axis represents CpG sites.

FIG. 28A depicts a plot correlating % methylation calls obtained using a library described herein vs. a comparator array. X and Y axis are labeled 0.00-1.00 at 0.25 intervals.

FIG. 28B depicts a plot correlating % methylation calls obtained using a library described herein vs. a comparator array. The data set has been down sampled to 100,000 sites for clarity. X and Y axis are labeled 0.00-1.00 at 0.25 intervals.

FIG. 29 depicts a plot of density vs. methylation calls (%) for a library described herein (“methylome NGS”) vs. an array-based comparator. The y-axis is labeled 0-4 at 1 unit intervals. The x-axis is labeled methylation calls (%) 0.00-1.00 at 0.25 unit intervals. Vertical dashed lines correspond to the array method (short dash) and methylome NGS (long dash).

FIG. 30A depicts a plot of methylation percent vs. genomic position for both libraries described herein and an array comparator. The y-axis is labeled methylation 0-100%; the x-axis is labeled CpG methylation sites from 9,000 to 10,000 at 250 base intervals. The dashed box represents an expanded view shown in FIG. 30B.

FIG. 30B depicts an expanded plot of methylation percent vs. genomic position for both libraries described herein and an array comparator. The y-axis is labeled methylation 0-100% at 10% intervals; the x-axis is labeled CpG methylation sites from 9,100 to 9,250 at 50 base intervals.

FIG. 31A depicts a plot of discordance of methylation percent by method (methylome NGS vs. array-based comparator). The y-axis is labeled count from 0-150,000 at 50,000 unit intervals. The x-axis is labeled 0.0%-100% at 10 unit intervals. The mean abs DELTA=8.45%, represented by the dashed line. For each set of bars, the left bar represents the array comparator and the right bar represents methylome-NGS.

FIG. 31B depicts a plot of discordance of methylation percent. The y-axis is labeled count from 0-3×10⁵ at 10⁵ unit intervals. The x-axis is labeled abs_DELTA from 0%-100% at 10 unit intervals. The mean abs_DELTA is represented by the dashed line.

FIG. 32A depicts a plot of methylation calls for the BRCA1 promoter region: ENCODE Accession: EH38E1864119 (chr17:43125311-43125660) for both array-based comparator and methylome NGS methods.

FIG. 32B depicts visual representations of per-site methylation trends. Merge context=metrics from individual cytosines in a CpG to be merged, producing per-CpG metrics rather than per cytosine metrics. Gray reads represent +strand un-methylated C calls (blue) and methylated C calls (red). Green reads represent −strand un-methylated C calls (blue) and methylated C calls (red).

FIG. 33A depicts sample to sample correlations for eight sample replicates of data from methylation analysis of NA12878. The values represent Pearson correlations.

FIG. 33B depicts sample to sample plots from eight sample replicates of data using methylation analysis of NA12878. Each plot y-axis is labeled 0 to 100 at 20 unit increments, and each plot x-axis is labeled 0 to 100 at 25 unit increments.

FIG. 34 depicts a workflow for a methylation system comprising genomic DNA (or cfDNA), methylation controls (optional), NGS library preparation and enzymatic conversion (addition of adapters and barcodes), Target enrichment with blockers solution, universal blockers, methylation enhancer, converted libraries, custom methylation panel, and control panel (optional), and a next generation sequencing platform.

FIGS. 35A-35B depict Picard Metrics when using a Methylome Panel Libraries prepared from 200 ng of gDNA input (NA12878, Coriell) with the NEBNext EM-seq kit. Target enrichment was performed using 200 ng of gDNA library input, a 63° C. Fast Wash 1 Buffer temperature, and 16-hour hybridization reactions. Sequencing was performed with the Illumina NovaSeq platform, using NovaSeq V1.5 chemistry kits and 150 bp paired-end reads. Data was downsampled to various aligned coverage (100×, 150×, 200×, 250×) relative to probe territory and analyzed with BWA-meth/MethylDackel and Picard HsMetrics. Figure show common picard metrics (FIG. 35A), and the number of samples that can be multiplexed on a Novaseq 6000 S4 flow cell based on sequencing coverage depth (FIG. 35B).

FIG. 36A depicts eight Specific Levels of Methylation with 3 Distinct Numbers of CpG Sites per Sequence. Within each methylation level containing a unique sequence, the material is made up of either low (2, left series), medium (8, middle series) or high (18, right series) CpG sites. Each pool of controls contains different ratios of methylation levels in order to symbolize 0%, 3%, 6%, 12%, 25%, 50%, 75% or 100%. This is achieved by mixing unique sequences that are 0% methylated material (lighter color) and 100% methylated material (darker color).

FIG. 36B depicts Measured vs. Expected % Methylation of Each Methylation Level Using a Methylation Detection System. 1 ul Methylation controls were spiked into 200 ng gDNA then taken through NEBNext EM-seq library preparation and Targeted Methylation sequencing workflow. Sequencing was performed on an Illumina 550 instrument using 2×150 paired end reads. Quantitation of each methylation level was measured using a BWA-meth/methyldackel analysis workflow to determine the amount of methylation for each sequence.

FIG. 37A depicts Picard Metrics from cfDNA Captured with a Cancer Specific Methylation Panel Libraries were prepared from 10 ng or 30 ng cfDNA input (BioIVT) with the NEBNext EM-seq kit using standard adapters or methylated UMI adapters. Target enrichment was performed using the Alliance Pan Cancer Methylation Panel (Twist Bioscience). Sequencing was performed on a Illumina Nextseq 2000 platform, using 2×100 paired-end reads. Data was downsampled to 1000× aligned coverage relative to probe territory and analyzed with BWA-meth/MethylDackel and Picard HsMetrics. Consistent performance between UMI and non-UMI containing libraries can be seen across coverage uniformity, percent on-target rate, HS library size, and percent targets at 30× coverage.

FIG. 37B depicts mean Target Coverage using UMI for Deduplication Comparison of mean depth over panel targets using either position-based deduplication (as offered in GATK MarkDuplicates) or UMI-informed dupsmarking, which splits reads into families based on both duplex UMIs and mapping positions and then marks duplicates on the basis of this information. After dupsmarking with both strategies we measured the increase in mean-target coverage that could be obtained by avoiding collapse of coincidental library fragments.

FIG. 38 depicts an analysis workflow comprising alignment of reads to converted genome while preserving UMI information.

### DETAILED DESCRIPTION

Described herein are compositions and methods for analysis of post-transcriptional modifications. In some instances, post-transcriptional modifications comprise methylation. Methods and compositions described herein in some instances provide improved methylated base calling and sequencing results over array-based methods. Further described herein are hybridization compositions and methods. Hybridization and/or capture of specific sequence fragments from complex sample mixtures using polynucleotide probes in some instances comprises use of a blocking reagent. Traditionally, such blocking reagents (e.g., cot-1, salmon sperm, or other blocking reagent) comprise highly repetitive sequence regions and are employed to prevent hybridization of one or more polynucleotide probes to off-target regions. However, such reagents are not tuned to specific sample mixtures, and may result in lower efficiency for such sample mixtures. Additionally, isolation of suitable hybridization reagents from various organisms can be time consuming, expensive, and/or provide low purity reagents. Described herein are synthetic blocking libraries of polynucleotides configured to improve efficient and sequencing metrics of enrichment methods which provide advantages over use of traditional blocking reagents. Further described herein are synthetic blocking libraries which are configured to bind to polynucleotide samples which have been treated with reagents to identify post-transcriptional base modifications (e.g., bisulfite to identify methylation via C->T conversion). Blocking libraries described herein are in some instances used for any hybridization-based application.

### Definitions

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein, the terms “preselected sequence”, “predefined sequence” or “predetermined sequence” are used interchangeably. The terms mean that the sequence of the polymer is known and chosen before synthesis or assembly of the polymer. In particular, various aspects of the invention are described herein primarily with regard to the preparation of nucleic acids molecules, the sequence of the oligonucleotide or polynucleotide being known and chosen before the synthesis or assembly of the nucleic acid molecules.

The term nucleic acid encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. The length of polynucleotides, when provided, are described as the number of bases and abbreviated, such as nt (nucleotides), bp (bases), kb (kilobases), Mb (megabases) or Gb (gigabases).

Provided herein are methods and compositions for production of synthetic (i.e. de novo synthesized or chemically synthesizes) polynucleotides. In some instances, polynucleotides comprise polynucleotide probes. The term oligonucleic acid, oligonucleotide, oligo, and polynucleotide are defined to be synonymous throughout. Libraries of synthesized polynucleotides described herein may comprise a plurality of polynucleotides collectively encoding for one or more genes or gene fragments. In some instances, the polynucleotide library encodes for sense strands, antisense strands, or both sense strands and antisense strands of one or more sequences. In some instances, the polynucleotide library encodes for sequences to identify methylation patterns. In some instances, the polynucleotide library encodes for sequences to identify methylation patterns which reflect chemical changes to one or more methylated or unmethylated bases. In some instances, the polynucleotide library comprises coding or non-coding sequences. In some instances, the polynucleotide library encodes for a plurality of cDNA sequences. Reference gene sequences from which the cDNA sequences are based may contain introns, whereas cDNA sequences exclude introns. Polynucleotides described herein may encode for genes or gene fragments from an organism. Exemplary organisms include, without limitation, prokaryotes (e.g., bacteria), eukaryotes (e.g., mice, rabbits, humans, plants, fungi, and non-human primates, bovine, porcine), or viruses. In some instances, the polynucleotide library comprises one or more polynucleotides, each of the one or more polynucleotides encoding sequences for multiple exons. Each polynucleotide within a library described herein may encode a different sequence, i.e., non-identical sequence. In some instances, each polynucleotide within a library described herein comprises at least one portion that is complementary to sequence of another polynucleotide within the library. Polynucleotide sequences described herein may be, unless stated otherwise, comprise DNA or RNA. A polynucleotide library described herein may comprise at least 10, 20, 50, 100, 200, 500, 1,000, 2,000, 5,000, 10,000, 20,000, 30,000, 50,000, 100,000, 200,000, 500,000, 1,000,000, or more than 1,000,000 polynucleotides. A polynucleotide library described herein may have no more than 10, 20, 50, 100, 200, 500, 1,000, 2,000, 5,000, 10,000, 20,000, 30,000, 50,000, 100,000, 200,000, 500,000, or no more than 1,000,000 polynucleotides. A polynucleotide library described herein may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 polynucleotides. A polynucleotide library described herein may comprise about 370,000; 400,000; 500,000 or more different polynucleotides. A polynucleotide library described herein may comprise at least 100,000, 500,000, 1 million, 1.5 million, 2 million, 3, million, 4 million, 5 million, 6 million, 8 million, or at least 10 million polynucleotides. A polynucleotide library described herein may comprise about 100,000, 1 million, 1.5 million, 2 million, 3, million, 4 million, 5 million, 6 million, 8 million, or about 10 million polynucleotides. A polynucleotide library described herein may comprise 100,000-10 million, 100,000-5 million, 500,000-5 million, 1 million-5 million, 2 million-5 million, 3 million-10 million, 4 million-6 million, or 5 million to 10 million polynucleotides. In instances, polynucleotide are double stranded.

Methylation patterns (e.g., those targeted by polynucleotide/probe libraries described herein) may comprise one or more methylation features. In some instances, methylation features comprise CpG features. In some instances, methylation features comprise one or more of CpG islands, CpG shores, CpG shelves and CpG open seas. In some instances, methylation patterns comprise at least 25%, 50%, 60%, 70%, 75%, 80%, 85%, 90%, 95%, or at least 98% of all CpG sites in the human genome. In some instances, methylation patterns comprise 25%-100%, 25-80%, 25-75%, 25-50%, 40-95%, 40-80%, 50-90%, 50-95%, 50-80%, 75-100%, 75-95%, 75-90%, or 75-85% of all CpG sites in the human genome. Polynucleotides in some instances are configured to target (hybridize) to one or more regulatory features of a genome. In some instances, regulatory features are involved in methylation. In some instances, regulatory features comprise CTCF binding sites, enhancers, open chromatin regions, promoters or transcription factor binding sites. In some instances, a methylation pattern is associated with a disease or condition. In some instances, the disease is cancer. In some instances, polynucleotides are configured to bind to CpG sites associated with at least one cancer. In some instances, polynucleotides are configured to bind to CpG sites associated with at least 2, 3, 5, 10, 15, 20, 25, or at least 30 different cancers.

### Synthetic Blocking Libraries

Described herein are synthetic blocking libraries (or hybridization reagents) comprising polynucleotides (polynucleotide library). In some instances such blocking libraries are configured to reduce undesired hybridization to sequences in a complex sample mixture (e.g., genome or collection of genomes). In some instances, blocking libraries are configured to bind to modified genomes. In some instances, blocking libraries comprise at least one modification relative to a genomic DNA. In some instances the at least one modification comprises a different abundance of one or more polynucleotides relative to an abundance in the genome. In some instances, modified genomes comprise post-transcriptional modifications identified through a conversion process. In some instances, the post-transcriptional modification comprises methylation (e.g., 5-methylcytosine, 5-hydroxymethylcytosine, or other modification). In some instances, blocking libraries are configured to bind to samples from specific organisms, such as humans or plants. In some instances, organisms comprise highly repetitive genetic elements, such as those found in polyploid species.

Hybridization reagents used for blocking (including synthetic blocking libraries) may contain repetitive sequences. For example, cot-1 comprises a fraction of repetitive, rapidly annealing polynucleotides length 50-300 bases isolated from human placental DNA. Such sequences generally include Alu and Kpn family members. In some instances, a synthetic blocking library described herein has a c0t value (e.g., c0t−1). Such c0t values in some instances represent a DNA concentration (mol/L)×renaturation time (in see)×a buffer factor. Faster renaturation results in lower c0t values. Lower c0t values generally correspond with a sample having a higher number of repetitive sequences. In some instances, a blocking library described herein comprises a c0t value of no more than 3, 2.8, 2.5, 2.2, 2.0, 1.8, 1.6, 1.4, 1.3, 1.2, 1.1, 1.0. 0.8, or no more than 0.5. In some instances, a blocking library described herein comprises a c0t value of about 3, 2.8, 2.5, 2.2, 2.0, 1.8, 1.6, 1.4, 1.3, 1.2, 1.1, 1, 0.8, or about 0.5. In some instances, a blocking library described herein comprises a c0t value of 0.1-3, 0.2-3, 0.5-3, 0.5-2, 0.5-1.5, 0.8-1.5, 1-3, or 1-2. In some instances, c0t values for polynucleotides are measured by placing the polynucleotides in a buffer, heating until they denature, and then allowing the polynucleotides to cool and reanneal. In some instances, the reannealing process is monitored using spectroscopy or other method. In some instances, polynucleotides comprise no more than 10%, 9%, 8%, 7%, 6%, 5%, 4%, 3%, 2%, or no more than 1% repetitive sequences. In some instances, polynucleotides comprise 0.001-10%, 0.01-10%, 0.1-10%, 1-10%, 2-10%, 3-10%, 5-10%, 7-10%, 0.1-4%, 0.01-3%, 0.1-3%, 1%-3%, or 2%-10% repetitive sequences. In some instances, a repetitive sequence comprises at least 5, 10, 15, 20, 25, 30, 35, 50, 100, 200, or more than 500 bases repeated in a genome or polynucleotide library.

### Methylome Analysis

Analysis of the methylome may provide important information on biological processes for a given genomic sample. Provided herein are hybridization reagents (polynucleotide blocking libraries) configured to reduce off-target binding during hybridization methods (such as those where one or more bases are converted to other bases). In some instances, methylated bases in a genomic sample are identified by either (a) conversion of a methylated base to a different base, or (b) conversion of a non-methylated base to a different base. Such conversions in some instances are performed on whole genomes or genomic fragments. The resulting sequences are then compared to a reference sequence (obtained without conversion/treatment) to identify which bases are methylated. In some instances, a conversion method (or process) comprises treatment with a deamination reagent. In some instances, a conversion method comprises treatment with bisulfite. In some instances, a conversion method comprises treatment with a reagent to protect methylcytosines (e.g., TET2 for oxidation), followed by treatment with an enzyme to deaminate unprotected cytosines (e.g., APOBEC). Libraries provided herein in some instances are configured to bind with both treated and untreated genomic DNA. Additional reagents which differentiate methylated and non-methylated bases are also consistent with the methods disclosed herein. In some instances, unmethylated cytosines are converted to uracil. In some instances, PCR amplification of these uracil-containing modified genomes results in conversion of uracil to thymine. In some instances, methods described herein comprise fragmentation of a sample comprising nucleic acids (e.g., genomic DNA), A-tailing, ligation of universal adapters, methylation conversion (oxidation and deamination), and amplification/barcode addition. In some instances, the method further comprises sequencing. Samples in some instances comprise genomic DNA. In some instances, samples comprise cell-free DNA. In some instances, cell-free DNA comprises circulating tumor DNA (ctDNA). In some instances, ctDNA comprises fragments of 50-300, 50-250, 75-200, 100-150, 100-175, 125-200, 125-175, 150-200, 150-180, 160-180, 150-170 or 100-200 bases in length. In some instances, ctDNA comprises fragments having an average length of 50-300, 50-250, 75-200, 100-150, 100-175, 125-200, 125-175, 150-200, 150-180, 160-180, 150-170 or 100-200 bases in length.

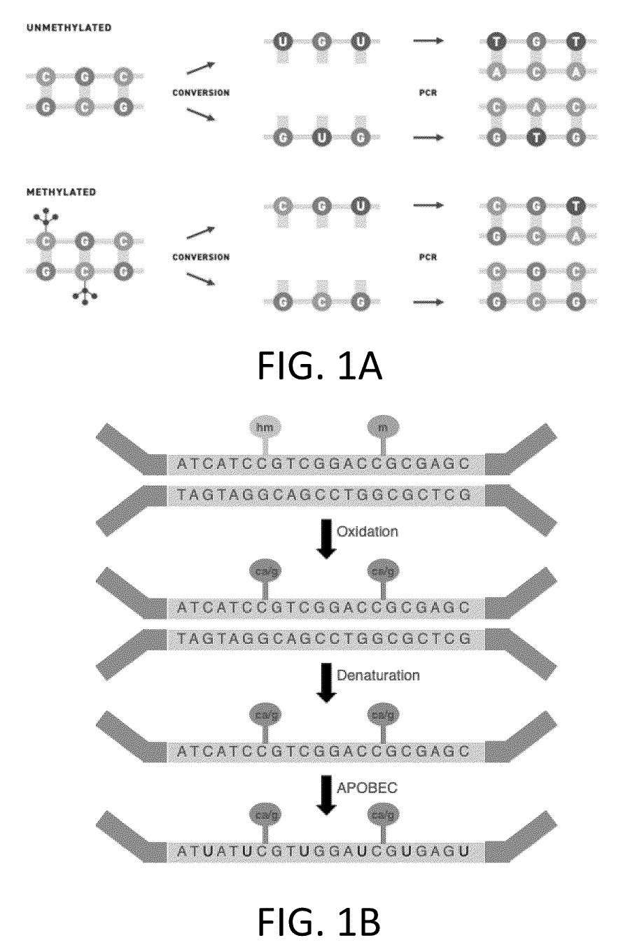

Polynucleotide libraries described herein may be used to capture or enrich all or portions of a nucleic acid sample comprising methylations (e.g., panels, probes). In some instances, polynucleotide libraries are used with synthetic polynucleotide blockers described herein. In some instances, polynucleotides are configured to hybridize with sense strand of a region to be enriched/captured, an antisense strand of a region to be enriched/captured, or both. In some instances, polynucleotides are configured to hybridize with a sequence corresponding to a “post” methylation conversion sequence (enzymatic or chemical). In some instances, a region may be targeted or enriched with polynucleotides targeting a “non-methylated” or “methylated” sequence. In some instances, a region may be targeted or enriched with polynucleotides targeting a treated and untreated genomic sample. In some instances, a region may be targeted or enriched with polynucleotides targeting a “unmethylated” or “methylated” sequence, and the reverse complement of each sequence (e.g., the antisense strand). This in some instances results in capture of both target nucleic acids comprising both “unmethylated” and “methylated” DNA. In some instances, a region is targeted or enriched by at least 2, 3, 4, or more than 4 different polynucleotides described herein. In some instances, a region is targeted or enriched by 3 or 4 polynucleotides described herein. In a non-limiting example, the sequences shown in left side of FIG. 1 are enriched by use of any one of the polynucleotides comprising the sequences on the right side (e.g., at least 1, 2, 3, 4, 5, 6, 7, or 8 sequences). In some instances, a region is targeted or enriched by 4 polynucleotides.

Any method which distinguishes methylated bases from non-methylated bases may be used with the methods described herein (conversion methods). In some instances, a method described herein comprises a conversion method. In some instances, unmethylated cytosines are converted to uracil with a reagent, such as bisulfite. In some instances, a conversion method comprises treatment with a reagent to protect methylcytosines (e.g., TET2, other enzyme or chemical other reagent for oxidation), followed by treatment with a reagent to deaminate unprotected cytosines (e.g., APOBEC, other deamination enzyme, or deamination chemical reagent). In some instances, a conversion method comprises a TET family enzyme. In some instances, a conversion method comprises a TET family enzyme and a chemical reagent. In some instances, a conversion method comprises a TET family enzyme and a chemical reagent configured to deaminate. In some instances, a conversion method comprises Tet-assisted pyridine borane sequencing (TAPS), TAPSβ, or Chemical-assisted pyridine borane sequencing (CAPS). In some instances, a conversion method comprises treatment with an oxidizing reagent that oxidizes both 5-methylcytosine (5mC) and 5-hydroxymethylcytosine (5hmC) to 5-carboxylcytosine (5caC) (e.g., ten-eleven translocation (Tet1) or other oxidizing enzyme or reagent). In some instances, a conversion method comprises treatment with a reducing reagent (e.g., pyridine borane) which reduces 5caC to dihydrouracil, a uracil derivative that a polymerase (PCR or isothermal polymerase) converts to thymine. In some instances, a conversion method comprises treatment with a transferase which labels 5hmC with a sugar. In some instances, a conversion method comprises treatment with 0-glucosyltransferase which labels 5hmC with glucose and protects 5hmC from the oxidation and reduction reactions. In some instances, a conversion method comprises treatment with an oxidizing agent which specifically oxidizes 5hmC (e.g., potassium perruthenate, other oxidizing enzyme or chemical reagent). In some instances, enzymes or chemical reagents are substituted to mimic or provide the same reactivity (e.g., chemical oxidant replaced with oxidizing enzyme). In some instances, one or more enzymes in a conversion method is replaced by one or more chemical reagents. In some instances, one or more chemical reagents in a conversion method is replaced by one or more enzymes. In some instances, two or more conversion methods are used to differentiate locations and types of base modifications. In some instances, hybridization reagents do not comprise 5-methylcytosine or 5-hydroxymethylcytosine.

Hybridization reagents for blocking may comprise polynucleotides having sequences (genomic sequences) derived from genomic DNA. In some instances, the genomic sequence is derived from placental DNA. In some instances, at least 25%, 50%, 75%, 80%, 85%, 90%, 95%, 97%, or at least 99% of the cytosine bases of the plurality of polynucleotides are replaced with uracil or thymine relative to the reference sequence. In some instances, 20-95%, 25-50%, 25-75%, 25-80%, 50-85%, 50-90%, 60-95%, 80-97%, or 25-99% of the cytosine bases of the plurality of polynucleotides are replaced with uracil or thymine relative to the reference sequence. In some instances, at least 25%, 50%, 75%, 80%, 85%, 90%, 95%, 97%, or at least 99% of the cytosine bases are not methylated in the genomic DNA. In some instances, 25-95%, 25-75%, 25-50%, 50-75%, 50-80%, 50-85%, 50-90%, 75-95%, 25-97%, or 25-99% of the cytosine bases are not methylated in the genomic DNA.

Provided herein are systems for methylation (or methylome) detection/analysis. In some instances a methylome detection system comprises a polynucleotide probe library described herein configured to positions of potential methylation; one or more enzymes configured to distinguish methylated from unmethylated bases; a plurality of sample nucleic acids; and optionally a methylation control. In some instances, the methylation control comprises a plurality of polynucleotides comprising at least eight different amounts of methylation. In some instances, the methylation control comprises at least 8, 16, 24, 36, 48, 64, or at least 128 different sequences. In some instances, the methylation control comprises at least 8, 16, 24, 36, 48, 64, or at least 128 different sequences having no homology to sequences in the Refseq database. In some instances, the methylation control comprises polynucleotides having an average length of 100-300, 125-250, 125-200, 125-150, 150-200, 150-190, 160-180, or 150-175 bases. In some instances, the methylation control comprises polynucleotides having an average length of about 167 bases. In some instances, the sample nucleic acids comprise cell-free DNA. In some instances, the plurality of genomic fragments comprise ctDNA.

### Design of Synthetic Blocking Libraries

Described herein are synthetic blocking derived from a source sequence. Source sequences (e.g., “input genome”) in some instances comprise one or more sequences which interfere or negatively affect an enrichment/capture process during hybridization. In some instances, off-target reads identified from a previous experiment are used as source sequences. In some instances, source sequences are generated from a genome which has been modified (e.g., bisulfite/enzymatic conversion). In some instances, source sequences are generated directly from a reference genome. In some instances, use of synthetic blocking libraries results in improved sequencing outcomes compared to naturally derived blocking agents (e.g., blocking reagents obtained from the organism). Synthetic blocking libraries in some instances are generated from both positive and negative strands of a source sequence. However, the blocking polynucleotide in the library corresponding to each strand need not be identical. In some instances, one or more computer algorithm steps are performed to generate sequences for the polynucleotides comprising a synthetic blocking library. Source sequences are in some instances derived from any organism, including but not limited to rodents (e.g., mouse, rat, hamster), porcine, bovine, primates (monkey, human), bacteria, fungi, plant, virus, or other organism. In some instances, source sequences are derived from plants of agricultural origin, such as grasses (wheat, barley, com, rice), fruits, vegetables, or other agricultural plant. In some instances, source sequences are derived from food crops. In some instances, food crops include but are not limited to wheat, onion, barley, rye, oat, com, soybeans, rice, sweet potato, cassava, yam, plantain, or potato. In some instances, the organism is diploid. In some instances, the organism is polyploid. In some instances, the organism comprises at least 3, 4, 5, 6, 7, 8, 9, 10, 20, 30, 40, 50, or 60 complete sets of chromosomes.

In a first step, computer algorithms may be used to generate sequences for synthetic blocking library designs. In some instances, sequences to be blocked in the source sequences are determined (e.g., repetitive, low complexity, or specific types of sequences) using software to count k-mers of a given size along the source sequences. In some instances, k-mers which are oligonucleotide sequences of a given length in the genome are currently computed for all sequences of a given length found within the input genome. In some instances, the given length is about 5, 10, 15, 20, 25, 30, 35, 40, 45, 50 or about 55 bases. In some instances, the given length is 5-50, 10-40, 10-50, 15-50, 15-40, 20-40, or 25-50 bases. In some instances, k-mers are computed to enable collapsing k-mers that differ by one or more mutations into a single “k-mer” entity for which all counts are added together, and/or to include counts for k-mers different or varying size.

In a second step, k-mers may be filtered. In some instances, k-mers are filtered for those with at least N=a given number of copies in the input genome. N is tuned or includes different numbers of copies, or various different k-mer sizes depending on application (e.g., lower copy numbers for large regions that still yield off-target at values of N<200, e.g., N=2 or higher). In some instances, N is 2, 5, 10, 20, 50, 80, 100, 120, 150, 180, 200, 250, 300, 400, or about 500. In some instances, N is 2-200, 2-250, 5-100, 50-300, 100-300, 200-300 or 150-300. In some instances, filtering enables tuning a desired stringency and/or total sequences manufactured. In some instances, k-mers are clustered using a variety sequence clustering algorithms to reduce the number of targets.

In a third step, k-mers may be mapped. In some instances, k-mers are mapped back to the source sequence (e.g., genome) through alignment to determine original location. In some instances, the original k-mer software or inhouse software was used to scan the source sequence and determine the exact origin in the input genome of k-mer sequences kept from the previous step. In some instances, tolerance for mismatches is adjusted (edit distance, difference of 0 or more variations in the genome sequence relative to the k-mer), size, or other criteria for determining a match that reduce or generalize the specificity to determined sequences. In some instances, the edit distance is about 0, 1, 2, 3, 4, 5, 10, or more than 10 variations. In some instances, a variation comprises a substitution (e.g., A>G, A>C, A>T, G>A, etc.), insertion (e.g., A>AT, G>CT, etc.), or deletion (AT>T, GC>C, etc.). In other instances, mutation tolerance comprises variant tolerance. In some instances methods described herein analyze variation in a genome in addition to mutation.

Polynucleotides which form the synthetic blocking library may be of any given length. In some instances, a given length for the polynucleotides to be synthesized are designed, capturing the sequence centered the middle of the original k-mer location using the input source sequences. In some instances, In some instances, this was adjusted by varying the size or mix of sizes of oligonucleotides synthesized which can modulate the strength, or the uniformity of the effect for different type of sequences. In some instances, additional steps included one or more of clustering or additionally filtering sequences to reduce number of targets, improving balancing of effect across all or subsets of the sources of off-target sequences, different nucleotide content across sequences, or other metrics which vary across the original population of detected k-mers or their relation to each other. In some instances, polynucleotides in the blocking library are about 50, 80, 90, 100, 110, 120, 130, 140, 150, 170, 190, 200, or about 300 bases in length. In some instances, polynucleotides in the blocking library are no more than 50, 80, 90, 100, 110, 120, 130, 140, 150, 170, 190, 200, or no more than 300 bases in length. In some instances, polynucleotides in the blocking library are at least 50, 80, 90, 100, 110, 120, 130, 140, 150, 170, 190, 200, or at least 300 bases in length. In some instances, polynucleotides in the blocking library comprise an average length of 50-300, 75-300, 100-200, 75-150 75-200, 100-150, or 80-150 bases. In some instances, polynucleotides in the blocking library are 50-300, 75-300, 100-200, 75-150 75-200, 100-150, or 80-150 bases in length. In some instances, synthetic blocking libraries comprise at least 1000, 2000, 5000, 10,000, 20,000, 50,000, 100,000, or at least 200,000 polynucleotides. In some instances, synthetic blocking libraries comprise about 1000, 2000, 5000, 10,000, 20,000, 50,000, 100,000, or about 200,000 polynucleotides. In some instances, synthetic blocking libraries comprise 1000-10,000, 5000-10,000, 10,000-100,000, 50,000-500,000, or 250,000-1 million polynucleotides. In some instances polynucleotides comprise a universal primer region. In some instances, each of the plurality of polynucleotides is present in an amount within 10%, 20%, 50%, 100%, 200%, 500%, 1000%, 10,000% or 100,000% of the mean representation.

### Universal Adapters

Provided herein are universal adapters. In some instances, the universal adapters disclosed herein may comprise a universal polynucleotide adapter comprising a first strand and a second strand. In some instances, a first strand comprises a first primer binding region, a first non-complementary region, and a first yoke region. In some instances, a second strand comprises a second primer binding region, a second non-complementary region, and a second yoke region. In some instances, a primer binding region allows for PCR amplification of a polynucleotide adapter. In some instances, a primer binding region allows for PCR amplification of a polynucleotide adapter and concurrent addition of one or more barcodes to the polynucleotide adapter. In some instances, the first yoke region is complementary to the second yoke region. In some instances, the first non-complementary region is not complementary to the second non-complementary region. In some instances, the universal adapter is a Y-shaped or forked adapter. In some instances, one or more yoke regions comprise nucleobase analogues that raise the Tm between a first yoke region and a second yoke region. Primer binding regions as described herein may be in the form of a terminal adapter region of a polynucleotide. In some instances, a universal adapter comprises one index sequence. In some instances, a universal adapter comprises one unique molecular identifier. In some instances, universal adapters are configured for use with barcoded primers, wherein after ligation, barcoded primers are added via PCR.

A universal (polynucleotide) adapter may be shortened relative to a typical barcoded adapter (e.g., full-length “Y adapter”). For example, a universal adapter strand is 20-45 bases in length. In some instances, a universal adapter strand is 25-40 bases in length. In some instances, a universal adapter strand is 30-35 bases in length. In some instances, a universal adapter strand is no more than 50 bases in length, no more than 45 bases in length, no more than 40 bases in length, no more than 35 bases in length, no more than 30 bases in length, or no more than 25 bases in length. In some instances, a universal adapter strand is about 25, 27, 30, 32, 34, 36, 38, 40, 42, 44, 46, 48, 50, 52, 54, 56, 58, or about 60 bases in length. In some instances, a universal adapter strand is about 60 base pairs in length. In some instances, a universal adapter strand is about 58 base pairs in length. In some instances, a universal adapter strand is about 52 base pairs in length. In some instances, a universal adapter strand is about 33 base pairs in length.

A universal adapter may be modified to facilitate ligation with a sample polynucleotide. For example, the 5′ terminus is phosphorylated. In some instances, a universal adapter comprises one or more non-native nucleobase linkages such as a phosphorothioate linkage. For example, a universal adapter comprises a phosphorothioate between the 3′ terminal base, and the base adjacent to the 3′ terminal base. A sample polynucleotide in some instances comprises nucleic acid from a variety of sources, such as DNA or RNA of human, bacterial, plant, animal, fungal, or viral origin. An adapter-ligated sample polynucleotide in some instances comprises a sample polynucleotide (e.g., sample nucleic acid) with adapters universal adapters ligated to both the 5′ and 3′ end of the sample polynucleotide to form an adapter-ligated polynucleotide. A duplex sample polynucleotide comprises both a first strand (forward) and a second strand (reverse).

Universal adapters may contain any number of different nucleobases (DNA, RNA, etc.), nucleobase analogues, or non-nucleobase linkers or spacers. For example, an adapter comprises one or more nucleobase analogues or other groups that enhance hybridization (Tm) between two strands of the adapter. In some instances, nucleobase analogues are present in the yoke region of an adapter. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), xenonucleic acids (XNAs) morpholino backbone-modified bases, minor grove binders (MGBs), spermine, G-clamps, or a anthraquinone (Uaq) caps. In some instances, adapters comprise one or more nucleobase analogues selected from Table 1.

TABLE 1

| Base | A | T | G | C | U |
|---|---|---|---|---|---|
| Locked Nucleic Acid (LNA) | 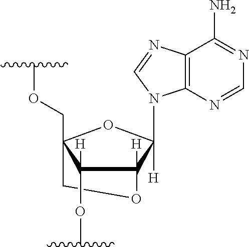 [SMILES: *OC[C@@]12COC([C@H](n3cnc4c(N)ncnc43)O1)[C@H]2O*] | 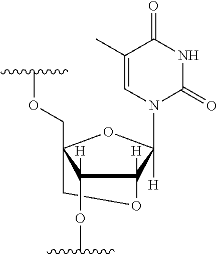 [SMILES: *OC[C@@]12COC([C@H](n3cc(C)c(=O)[nH]c3=O)O1)[C@H]2O*] | 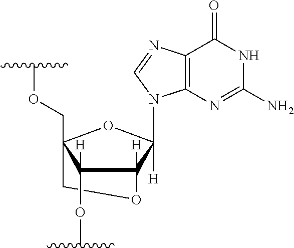 [SMILES: *OC[C@@]12COC([C@H](n3cnc4c(=O)[nH]c(N)nc43)O1)[C@H]2O*] | 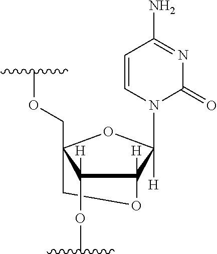 [SMILES: *OC[C@@]12COC([C@H](n3ccc(N)nc3=O)O1)[C@H]2O*] | 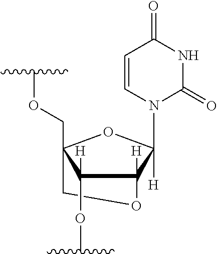 [SMILES: *OC[C@@]12COC([C@H](n3ccc(=O)[nH]c3=O)O1)[C@H]2O*] |
| Bridged Nucleic Acid* (BNA) | 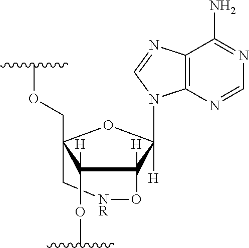 | 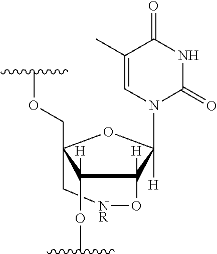 | 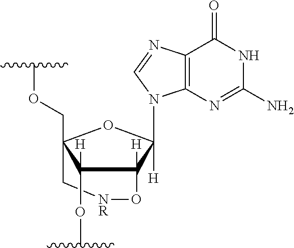 | 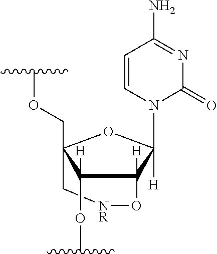 | 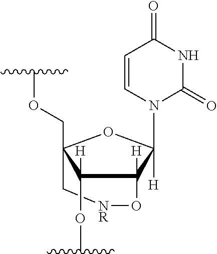 |

*R is H or Me.

Universal adapters may comprise any number of nucleobase analogues (such as LNAs or BNAs), depending on the desired hybridization Tm. For example, an adapter comprises 1 to 20 nucleobase analogues. In some instances, an adapter comprises 1 to 8 nucleobase analogues. In some instances, an adapter comprises at least 1, 2, 3, 4, 5, 6, 7, 8, 9, 10, 11, 12, or at least 12 nucleobase analogues. In some instances, an adapter comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the adapter. For example, an adapter comprises at least 1%, 2%, 5%, 10%, 12%, 18%, 24%, 30%, or more than 30% nucleobase analogues. In some instances, adapters (e.g., universal adapters) described herein comprise methylated nucleobases, such as methylated cytosine.

### Barcoded Primers

Polynucleotide primers may comprise defined sequences, such as barcodes (or indices). Barcodes can be attached to universal adapters, for example, using PCR and barcoded primers to generate barcoded adapter-ligated sample polynucleotides. Primer binding sites, such as universal primer binding sites, facilitate simultaneous amplification of all members of a barcode primer library, or a subpopulation of members. In some instances, a primer binding site comprises a region that binds to a flow cell or other solid support during next generation sequencing. In some instances, a barcoded primer comprises a P5 (5′-AATGATACGGCGACCACCGA-3′) or P7 (5′-CAAGCAGAAGACGGCATACGAGAT-3′) sequence. In some instances, primer binding sites are configured to bind to universal adapter sequences, and facilitate amplification and generation of barcoded adapters. In some instances, barcoded primers are no more than 60 bases in length. In some instances, barcoded primers are no more than 55 bases in length. In some instances, barcoded primers are 50-60 bases in length. In some instances, barcoded primers are about 60 bases in length. In some instances, barcodes described herein comprise methylated nucleobases, such as methylated cytosine.

The number of unique barcodes available for a barcode set (collection of unique barcodes or barcode combinations configured to be used together to unique define samples) may depend on the barcode length. In some instances, a Hamming distance is defined by the number of base differences between any two barcodes. In some instances, a Levenshtein distance is defined by the number changes needed to change one barcode into another (insertions, substitutions, or deletions). In some instances, barcode sets described herein comprise a Levenshtein distance of at least 2, 3, 4, 5, 6, 7, or at least 8. In some instances, barcode sets described herein comprise a Hamming distance of at least 2, 3, 4, 5, 6, 7, or at least 8.

Barcodes may be incorrectly associated with a different sample than they were assigned. In some instances, incorrect barcodes are occur from PCR errors (e.g., substitution) during library amplification. In some instances, entire barcodes “hop” or are transferred from one sample polynucleotide to another. Such transfers in some instances result from cross-contamination of free adapters or primers during a library generation workflow. In some instances a group of barcodes (barcode set) is chosen to minimize “barcode hopping”. In some instances, barcode hopping (for a single barcode) for a barcode set described herein is no more than 7%, 5%, 4%, 3%, 2%, 1%, 0.5%, or no more than 0.1%. In some instances, barcode hopping (for a single barcode) for a barcode set described herein is 0.1-6%, 0.1-5%, 0.2-5%, 0.5-5%, 1-7%, 1-5%, or 0.5-7%. In some instances, barcode hopping (for two barcodes) for a barcode set described herein is no more than 0.7%, 0.5%, 0.4%, 0.3%, 0.2%, 0.1%, 0.05%, or no more than 0.1%. In some instances, barcode hopping (for two barcodes) for a barcode set described herein is 0.01-0.6%, 0.01-0.5%, 0.02-0.5%, 0.05-0.5%, 0.1-0.7%, 0.1-0.5%, or 0.05-0.7%.

Barcoded primers comprise one or more barcodes. In some instances, the barcodes are added to universal adapters through PCR reaction. Barcodes are nucleic acid sequences that allow some feature of a polynucleotide with which the barcode is associated to be identified. In some instances, a barcode comprises an index sequence. In some instances, index sequences allow for identification of a sample, or unique source of nucleic acids to be sequenced. A barcode or combination of barcodes in some instances identifies a specific patient. A barcode or combination of barcodes in some instances identifies a specific sample from a patient among other samples from the same patient. After sequencing, the barcode (or barcode region) provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, a barcode is positioned on the 5′ and the 3′ sides of a sample polynucleotide. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, or more 512 barcoded libraries are used. In some instances, at least 400, 500, 800, 1000, 2000, 5000, 10,000, 12,000, 15,000, 18,000, 20,000, or at 25,000 barcodes are used. Barcoded primers or adapters may comprise unique molecular identifiers (UMI). Such UMIs in some instances uniquely tag all nucleic acids in a sample. In some instances, at least 60%, 70%, 80%, 90%, 95%, or more than 95% of the nucleic acids in a sample are tagged with a UMI. In some instances, at least 85%, 90%, 95%, 97%, or at least 99% of the nucleic acids in a sample are tagged with a unique barcode, or UMI. Barcoded primers in some instances comprise an index sequence and one or more UMI. UMIs allow for internal measurement of initial sample concentrations or stoichiometry prior to downstream sample processing (e.g., PCR or enrichment steps) which can introduce bias. In some instances, UMIs comprise one or more barcode sequences. In some instances, each strand (forward vs. reverse) of an adapter-ligated sample polynucleotide possesses one or more unique barcodes. Such barcodes are optionally used to uniquely tag each strand of a sample polynucleotide. In some instances, a barcoded primer comprises an index barcode and a UMI barcode. In some instances, after amplification with at least two barcoded primers, the resulting amplicons comprise two index sequences and two UMIs. In some instances, after amplification with at least two barcoded primers, the resulting amplicons comprise two index barcodes and one UMI barcode. In some instances, each strand of a universal adapter-sample polynucleotide duplex is tagged with a unique barcode, such as a UMI or index barcode. In some instances, UMIs are present on one or more adapters ligated to sample nucleic acids. In some instances, adapters comprise at least one methylated base. In some instances, adapters comprise at 1, 2, 3, 4, 5, 10, or at least 15 methylated base. In some instances, all cytosines in the adapter are methylated.

Barcoded primers in a library comprise a region that is complementary to a primer binding region on a universal adapter. For example, universal adapter binding region is complementary to primer region of the universal adapter, and universal adapter binding region is complementary to primer region of the universal adapter. Such arrangements facilitate extension of universal adapters during PCR, and attach barcoded primers. In some instances, the Tm between the primer and the primer binding region is 40-65 degrees C. In some instances, the Tm between the primer and the primer binding region is 42-63 degrees C. In some instances, the Tm between the primer and the primer binding region is 50-60 degrees C. In some instances, the Tm between the primer and the primer binding region is 53-62 degrees C. In some instances, the Tm between the primer and the primer binding region is 54-58 degrees C. In some instances, the Tm between the primer and the primer binding region is 40-57 degrees C. In some instances, the Tm between the primer and the primer binding region is 40-50 degrees C. In some instances, the Tm between the primer and the primer binding region is about 40, 45, 47, 50, 52, 53, 55, 57, 59, 61, or 62 degrees C.

### Hybridization Blockers

Blockers may contain any number of different nucleobases (DNA, RNA, etc.), nucleobase analogues (non-canonical), or non-nucleobase linkers or spacers. In some instances, blockers comprise universal blockers. Such blockers may in some instances are described as a “set”, wherein the set comprises two or more blockers configured to prevent unwanted interactions with the same adapter sequence. In some instances, universal blockers prevent adapter-adapter interactions independent of one or more barcodes present on at least one of the adapters. For example, a blocker comprises one or more nucleobase analogues or other groups that enhance hybridization (Tm) between the blocker and the adapter. In some instances, a blocker comprises one or more nucleobases which decrease hybridization (Tm) between the blocker and the adapter (e.g., “universal” bases). In some instances, a blocker described herein comprises both one or more nucleobases which increase hybridization (Tm) between the blocker and the adapter and one or more nucleobases which decrease hybridization (Tm) between the blocker and the adapter.

Described herein are hybridization blockers comprising one or more regions which enhance binding to targeted sequences (e.g., adapter), and one or more regions which decrease binding to target sequences (e.g., adapter). In some instances, each region is tuned for a given desired level of off-bait activity during target enrichment applications. In some instances, each region can be altered with either a single type of chemical modification/moiety or multiple types to increase or decrease overall affinity of a molecule for a targeted sequence. In some instances, the melting temperature of all individual members of a blocker set are held above a specified temperature (e.g., with the addition of moieties such as LNAs and/or BNAs). In some instances, a given set of blockers will improve off bait performance independent of index length, independent of index sequence, and independent of how many adapter indices are present in hybridization.

Blockers may comprise moieties which increase and/or decrease affinity for a target sequencing, such as an adapter. In some instances, such specific regions can be thermodynamically tuned to specific melting temperatures to either avoid or increase the affinity for a particular targeted sequence. This combination of modifications is in some instances designed to help increase the affinity of the blocker molecule for specific and unique adapter sequence and decrease the affinity of the blocker molecule for repeated adapter sequence (e.g., Y-stem annealing portion of adapter). In some instances, blockers comprise moieties which decrease binding of a blocker to the Y-stem region of an adapter. In some instances, blockers comprise moieties which decrease binding of a blocker to the Y-stem region of an adapter, and moieties which increase binding of a blocker to non-Y-stem regions of an adapter.

Blockers (e.g., universal blockers) and adapters may form a number of different populations during hybridization. In a population ‘A’ in some instances comprises blockers correctly bound to non-index regions of the adapters. In a population ‘B’, a region of the blockers is bound to the “yoke” region of the adapter, but a remaining portion of the blocker does not bind to an adjacent region of the adapter. In a population ‘C’, two blockers unproductively dimerize. In a population ‘D’, blockers are unbound to any other nucleic acids. In some instances, when the number of DNA modifications that decrease affinity in the Y-stem annealing region of the blocker are increased, the populations ‘A’ & ‘D’ dominate and either have the desired or minimal effect. In some instances, as the number of DNA modifications that decrease affinity in the Y-stem annealing region of the blocker are decreased, the populations ‘B’ & ‘C’ dominate and have undesired effects where daisy-chaining or annealing to other adapters can occur (‘B’) or sequester blockers where they are unable to function properly (‘C’).

The index on both single or dual index adapter designs may be either partially or fully covered by universal blockers that have been extended with specifically designed DNA modifications to cover adapter index bases. In some instances, such modifications comprise moieties which decrease annealing to the index, such as universal bases. In some instances, the index of a dual index adapter is partially covered (or is overlapped) by one or more blockers. In some instances, the index of a dual index adapter is fully covered by one or more blockers. In some instances, the index of a single index adapter is partially covered by one or more blockers. In some instances, the index of a single index adapter is fully covered by one or more blockers. In some instances, a blocker overlaps an index sequence by at least 1, 2, 3, 4, 5, 6, 7, 8, 9, 10, 11, 12, 13, 14, 15, 20 or more than 20 bases. In some instances, a blocker overlaps an index sequence by no more than 1, 2, 3, 4, 5, 6, 7, 8, 9, 10, 11, 12, 13, 14, 15, 20, or no more than 25 bases. In some instances, a blocker overlaps an index sequence by about 1, 2, 3, 4, 5, 6, 7, 8, 9, 10, 11, 12, 13, 14, 15, 20 or about 30 bases. In some instances, a blocker overlaps an index sequence by 1-5, 1-3, 2-5, 2-8, 2-10, 3-6, 3-10, 4-10, 4-15, 1-4 or 5-7 bases. In some instances, a region of a blocker which overlaps an index sequences comprises at least one 2-deoxyinosine or 5-nitroindole nucleobase.

One or two blockers may overlap with an index sequence present on an adapter. In some instances, one or two blockers combined overlap with at least 1, 2, 3, 4, 5, 6, 7, 8, 9, 10, 11, 12, 13, 14, 15, 20 or more than 20 bases of the index sequence. In some instances, one or two blockers combined overlap with no more than 1, 2, 3, 4, 5, 6, 7, 8, 9, 10, 11, 12, 13, 14, 15, 20 or no more than 20 bases of the index sequence. In some instances, one or two blockers combined overlap with about 1, 2, 3, 4, 5, 6, 7, 8, 9, 10, 11, 12, 13, 14, 15, 20 or about 20 bases of the index sequence. In some instances, one or two blockers combined overlap by 1-5, 1-3, 2-5, 2-8, 2-10, 3-6, 3-10, 4-10, 4-15, 1-4 or 5-7 bases of the index sequence. In some instances, a region of a blocker which overlaps an index sequences comprises at least one 2-deoxyinosine or 5-nitroindole nucleobase.

In a first arrangement, the length of the adapter index overhang may be varied. When designed from a single side, the adapter index overhang can be altered to cover from 0 to n of the adapter index bases from either side of the index. This allows for the ability to design such adapter blockers for both single and dual index adapter systems.

In a second arrangement, the adapter index bases are covered from both sides. When adapter index bases are covered from both sides, the length of the covering region of each blocker can be chosen such that a single pair of blockers is capable of interacting with a range of adapter index lengths while still covering a significant portion of the total number of index bases. As an example, take two blockers that have been designed with 3 bp overhangs that cover the adapter index. In the context of 6 bp, 8 bp, or 10 bp adapter index lengths, these blockers will leave 0 bp, 2 bp, or 4 bp exposed during hybridization, respectively.

In a third arrangement, modified nucleobases are selected to cover index adapter bases. Examples of these modifications that are currently commercially available include degenerate bases (i.e., mixed bases of A, T, C, G), 2′-deoxylnosine, & 5-nitroindole.

In a forth arrangement, blockers with adapter index overhangs bind to either the sense (i.e., ‘top’) or anti-sense (i.e., ‘bottom’) strand of a next generation sequencing library.

In a fifth arrangement, blockers are further extended to cover other polynucleotide sequences (e.g., a poly-A tail added in a previous biochemical step in order to facilitate ligation or other method to introduce a defined adapter sequence, unique molecular identifier for bioinformatic assignment following sequencing, etc.) in addition to the standard adapter index bases of defined length and composition. These types of sequences can be placed in multiple locations of an adapter and in this case the most widely utilized case (i.e., unique molecular index next to the genomic insert) is presented. Other positions for the unique molecular identifier (e.g., next to adapter index bases) could also be addressed with similar approaches.

In a sixth arrangement, all of the previous arrangements are utilized in various combinations to meet a targeted performance metric for off-bait performance during target enrichment under specified conditions.

Blockers may comprise moieties, such as nucleobase analogues. Nucleobase analogues and other groups include but are not limited to locked nucleic acids (LNAs), bicyclic nucleic acids (BNAs), C5-modified pyrimidine bases, 2′-O-methyl substituted RNA, peptide nucleic acids (PNAs), glycol nucleic acid (GNAs), threose nucleic acid (TNAs), inosine, 2′-deoxylnosine, 3-nitropyrrole, 5-nitroindole, xenonucleic acids (XNAs) morpholino backbone-modified bases, minor grove binders (MGBs), spermine, G-clamps, or a anthraquinone (Uaq) caps. In some instances, nucleobase analogues comprise universal bases, wherein the nucleobase has a lower Tm for binding to a cognate nucleobase. In some instances, universal bases comprise 5-nitroindole or 2′-deoxyInosine. In instances, blockers comprise spacer elements that connect two polynucleotide chains. In some instances, blockers comprise one or more nucleobase analogues selected from Table 1. In some instances, such nucleobase analogues are added to control the Tm of a blocker. Blockers may comprise any number of nucleobase analogues (such as LNAs or BNAs), depending on the desired hybridization Tm. For example, a blocker comprises 20 to 40 nucleobase analogues. In some instances, a blocker comprises 8 to 16 nucleobase analogues. In some instances, a blocker comprises at least 1, 2, 3, 4, 5, 6, 7, 8, 9, 10, 11, 12, or at least 12 nucleobase analogues. In some instances, a blocker comprises about 1, 2, 3, 4, 5, 6, 7, 8, 9, 10, 11, 12, 13, 14, 15, or about 16 nucleobase analogues. In some instances, the number of nucleobase analogous is expressed as a percent of the total bases in the blocker. For example, a blocker comprises at least 1%, 2%, 5%, 10%, 12%, 18%, 24%, 30%, or more than 30% nucleobase analogues. In some instances, the blocker comprising a nucleobase analogue raises the Tm in a range of about 2° C. to about 8° C. for each nucleobase analogue. In some instances, the Tm is raised by at least or about 1° C., 2° C., 3° C., 4° C., 5° C., 6° C., 7° C., 8° C., 9° C., 10° C., 12° C., 14° C., or 16° C. for each nucleobase analogue. Such blockers in some instances are configured to bind to the top or “sense” strand of an adapter. Blockers in some instances are configured to bind to the bottom or “anti-sense” strand of an adapter. In some instances a set of blockers includes sequences which are configured to bind to both top and bottom strands of an adapter. Additional blockers in some instances are configured to the complement, reverse, forward, or reverse complement of an adapter sequence. In some instances, a set of blockers targeting a top (binding to the top) or bottom strand (or both) is designed and tested, followed by optimization, such as replacing a top blocker with a bottom blocker, or a bottom blocker with a top blocker. In some instances, a blocker is configured to overlap fully or partially with bases of an index or barcode on an adapter. A set of blockers in some instances comprise at least one blocker overlapping with an adapter index sequence. A set of blockers in some instances comprise at least one blocker overlapping with an adapter index sequence, and at least one blocker which does not overlap with an adapter sequence. A set of blockers in some instances comprise at least one blocker which does not overlap with a yoke region sequence. A set of blockers in some instances comprise at least one blocker which does not overlap with a yoke region sequence and at least one blocker which overlaps with a yoke region sequence. A sets of blockers in some instances comprises 2, 3, 4, 5, 6, 7, 8, 9, 10, or more than 10 blockers.

Blockers may be any length, depending on the size of the adapter or hybridization Tm. For example, blockers are 20 to 50 bases in length. In some instances, blockers are 25 to 45 bases, 30 to 40 bases, 20 to 40 bases, or 30 to 50 bases in length. In some instances, blockers are 25 to 35 bases in length. In some instances blockers are at least 25, 26, 27, 28, 29, 30, 31, 32, 33, 34, or at least 35 bases in length. In some instances, blockers are no more than 25, 26, 27, 28, 29, 30, 31, 32, 33, 34, or no more than 35 bases in length. In some instances, blockers are about 25, 26, 27, 28, 29, 30, 31, 32, 33, 34, or about 35 bases in length. In some instances, blockers are about 50 bases in length. A set of blockers targeting an adapter-tagged genomic library fragment in some instances comprises blockers of more than one length. Two blockers are in some instances tethered together with a linker. Various linkers are well known in the art, and in some instances comprise alkyl groups, polyether groups, amine groups, amide groups, or other chemical group. In some instances, linkers comprise individual linker units, which are connected together (or attached to blocker polynucleotides) through a backbone such as phosphate, thiophosphate, amide, or other backbone. In an exemplary arrangement, a linker spans the index region between a first blocker that each targets the 5′ end of the adapter sequence and a second blocker that targets the 3′ end of the adapter sequence. In some instances, capping groups are added to the 5′ or 3′ end of the blocker to prevent downstream amplification. Capping groups variously comprise polyethers, polyalcohols, alkanes, or other non-hybridizable group that prevents amplification. Such groups are in some instances connected through phosphate, thiophosphate, amide, or other backbone. In some instances, one or more blockers are used. In some instances, at least 4 non-identical blockers are used. In some instances, a first blocker spans a first 3′ end of an adaptor sequence, a second blocker spans a first 5′ end of an adaptor sequence, a third blocker spans a second 3′ end of an adaptor sequence, and a fourth blockers spans a second 5′ end of an adaptor sequence. In some instances a first blocker is at least 20, 21, 22, 23, 24, 25, 26, 27, 28, 29, 30, 31, 32, 33, 34, or at least 35 bases in length. In some instances a second blocker is at least 20, 21, 22, 23, 24, 25, 26, 27, 28, 29, 30, 31, 32, 33, 34, or at least 35 bases in length. In some instances a third blocker is at least 20, 21, 22, 23, 24, 25, 26, 27, 28, 29, 30, 31, 32, 33, 34, or at least 35 bases in length. In some instances a fourth blocker is at least 20, 21, 22, 23, 24, 25, 26, 27, 28, 29, 30, 31, 32, 33, 34, or at least 35 bases in length. In some instances, a first blocker, second blocker, third blocker, or fourth blocker comprises a nucleobase analogue. In some instances, the nucleobase analogue is LNA.

The design of blockers may be influenced by the desired hybridization Tm to the adapter sequence. In some instances, non-canonical nucleic acids (for example locked nucleic acids, bridged nucleic acids, or other non-canonical nucleic acid or analog) are inserted into blockers to increase or decrease the blocker's Tm. In some instances, the Tm of a blocker is calculated using a tool specific to calculating Tm for polynucleotides comprising a non-canonical amino acid. In some instances, a Tm is calculated using the Exiqon™ online prediction tool. In some instances, blocker Tm described herein are calculated in-silico. In some instances, the blocker Tm is calculated in-silico, and is correlated to experimental in-vitro conditions. Without being bound by theory, an experimentally determined Tm may be further influenced by experimental parameters such as salt concentration, temperature, presence of additives, or other factor. In some instances, Tm described herein are in-silico determined Tm that are used to design or optimize blocker performance. In some instances, Tm values are predicted, estimated, or determined from melting curve analysis experiments. In some instances, blockers have a Tm of 70 degrees C. to 99 degrees C. In some instances, blockers have a Tm of 75 degrees C. to 90 degrees C. In some instances, blockers have a Tm of at least 85 degrees C. In some instances, blockers have a Tm of at least 70, 72, 75, 77, 80, 82, 85, 88, 90, or at least 92 degrees C. In some instances, blockers have a Tm of about 70, 72, 75, 77, 80, 82, 85, 88, 90, 92, or about 95 degrees C. In some instances, blockers have a Tm of 78 degrees C. to 90 degrees C. In some instances, blockers have a Tm of 79 degrees C. to 90 degrees C. In some instances, blockers have a Tm of 80 degrees C. to 90 degrees C. In some instances, blockers have a Tm of 81 degrees C. to 90 degrees C. In some instances, blockers have a Tm of 82 degrees C. to 90 degrees C. In some instances, blockers have a Tm of 83 degrees C. to 90 degrees C. In some instances, blockers have a Tm of 84 degrees C. to 90 degrees C. In some instances, a set of blockers have an average Tm of 78 degrees C. to 90 degrees C. In some instances, a set of blockers have an average Tm of 80 degrees C. to 90 degrees C. In some instances, a set of blockers have an average Tm of at least 80 degrees C. In some instances, a set of blockers have an average Tm of at least 81 degrees C. In some instances, a set of blockers have an average Tm of at least 82 degrees C. In some instances, a set of blockers have an average Tm of at least 83 degrees C. In some instances, a set of blockers have an average Tm of at least 84 degrees C. In some instances, a set of blockers have an average Tm of at least 86 degrees C. Blocker Tm are in some instances modified as a result of other components described herein, such as use of a fast hybridization buffer and/or hybridization enhancer.

The molar ratio of blockers to adapter targets may influence the off-bait (and subsequently off-target) rates during hybridization. The more efficient a blocker is at binding to the target adapter, the less blocker is required. Blockers described herein in some instances achieve sequencing outcomes of no more than 20% off-target reads with a molar ratio of less than 20:1 (blocker:target). In some instances, no more than 20% off-target reads are achieved with a molar ratio of less than 10:1 (blocker:target). In some instances, no more than 20% off-target reads are achieved with a molar ratio of less than 5:1 (blocker:target). In some instances, no more than 20% off-target reads are achieved with a molar ratio of less than 2:1 (blocker:target). In some instances, no more than 20% off-target reads are achieved with a molar ratio of less than 1.5:1 (blocker:target). In some instances, no more than 20% off-target reads are achieved with a molar ratio of less than 1.2:1 (blocker:target). In some instances, no more than 20% off-target reads are achieved with a molar ratio of less than 1.05:1 (blocker:target).

The universal blockers may be used with panel libraries of varying size. In some embodiments, the panel libraries comprises at least or about 0.01, 0.02, 0.03, 0.04, 0.05, 0.06, 0.07, 0.08, 0.09, 1.0, 2.0, 4.0, 8.0, 10.0, 12.0, 14.0, 16.0, 18.0, 20.0, 22.0, 24.0, 26.0, 28.0, 30.0, 40.0, 50.0, 60.0, or more than 60.0 megabases (Mb).

Blockers as described herein may improve on-target performance. In some embodiments, on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% for various index designs. In some embodiments, the on-target performance is improved by at least or about 5%, 10%, 15%, 20%, 25%, 30%, 35%, 40%, 45%, 50%, 55%, 60%, 65%, 70%, 75%, 80%, 85%, 90%, 95%, or more than 95% is improved for various panel sizes.

### Hybridization Buffers

Any number of buffers may be used with the hybridization methods described herein. For example, a buffer comprises numerous chemical components, such as polymers, solvents, salts, surfactants, or other component. In some instances, hybridization buffers decrease the hybridization times (e.g., “fast” hybridization buffers) required to achieve a given sequencing result or level of quality. Such components in some instances lead to improved hybridization outcomes, such as increased on-target rate, improved sequencing outcomes (e.g., sequencing depth or other metric), or decreased off-target rates. Such components may be introduced at any concentration to achieve such outcomes. In some instances, buffer components are added in specific order. For example, water is added first. In some instances, salts are added after water. In some instances, salts are added after thickening agents and surfactants. In some instances, hybridization buffers such as “fast” hybridization buffers described herein are used in conjunction with universal blockers and liquid polymer additives. In some instances, use of fast hybridization buffers reduces hybridization times to no more than 4, 3, 2, 1, 0.5, 0.2, or 0.1 hours.

Hybridization buffers described herein may comprise solvents, or mixtures of two or more solvents. In some instances, a hybridization buffer comprises a mixture of two solvents, three solvents or more than three solvents. In some instances, a hybridization buffer comprises a mixture of an alcohol and water. In some instances, a hybridization buffer comprises a mixture of a ketone containing solvent and water. In some instances, a hybridization buffer comprises a mixture of an ethereal solvent and water. In some instances, a hybridization buffer comprises a mixture of a sulfoxide-containing solvent and water. In some instances, a hybridization buffer comprises a mixture of am amide-containing solvent and water. In some instances, a hybridization buffer comprises a mixture of an ester-containing solvent and water. In some instances, hybridization buffers comprise solvents such as water, ethanol, methanol, propanol, butanol, other alcohol solvent, or a mixture thereof. In some instances, hybridization buffers comprise solvents such as acetone, methyl ethyl ketone, 2-butanone, ethyl acetate, methyl acetate, tetrahydrofuran, diethyl ether, or a mixture thereof. In some instances, hybridization buffers comprise solvents such as DMSO, DMF, DMA, HMPA, or a mixture thereof. In some instances, hybridization buffers comprise a mixture of water, HMPA, and an alcohol. In some instances, two solvents are present at a 1:1, 1:2, 1:3, 1:4, 1:5, 1:8, 1:9, 1:10, 1:20, 1:50, 1:100, or 1:500 ratio.

Hybridization buffers described herein may comprise polymers. Polymers include but are not limited to thickening agents, polymeric solvents, dielectric materials, or other polymer. Polymers are in some instances hydrophobic or hydrophilic. In some instances, polymers are silicon polymers. In some instances, polymers comprise repeating polyethylene or polypropylene units, or a mixture thereof. In some instances, polymers comprise polyvinylpyrrolidone or polyvinylpyridine. In some instances, polymers comprise amino acids. For example, in some instances polymers comprise proteins. In some instances, polymers comprise casein, milk proteins, bovine serum albumin, or other protein. In some instances, polymers comprise nucleotides, for example, DNA or RNA. In some instances, polymers comprise polyA, polyT, Cot-1 DNA, or other nucleic acid. In some instances, polymers comprise sugars. For example, in some instances a polymer comprises glucose, arabinose, galactose, mannose, or other sugar. In some instances, a polymer comprises cellulose or starch. In some instances, a polymer comprises agar, carboxyalkyl cellulose, xanthan, guar gum, locust bean gum, gum karaya, gum tragacanth, gum Arabic. In some instances, a polymer comprises a derivative of cellulose or starch, or nitrocellulose, dextran, hydroxyethyl starch, ficoll, or a combination thereof. In some instances, mixtures of polymers are used in hybridization buffers described herein. In some instances, hybridization buffers comprise Denhardt's solution. Polymers described herein may be present at any concentration suitable for reducing off-target binding. Such concentrations are often represented as a percent by weight, percent by volume, or percent weight per volume. For example, a polymer is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a polymer is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a polymer is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a polymer is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a polymer is present at 0.005%-0.1%. In some instances, a polymer is present at 0.05%-0.1%. In some instances, a polymer is present at 0.005%-0.6%. In some instances, a polymer is present at 1%-30%, 5%-25%, 10%-30%, 15%-30%, or 1%-15%. Liquid polymers may be present as a percentage of the total reaction volume. In some instances, a polymer is about 10%, 20%, 30%, 40%, 50%, 60%, 75%, or about 90% of the total volume. In some instances, a polymer is at least 10%, 20%, 30%, 40%, 50%, 60%, 75%, or at least 90% of the total volume. In some instances, a polymer is no more than 10%, 20%, 30%, 40%, 50%, 60%, 75%, or no more than 90% of the total volume. In some instances, a polymer is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a polymer is 25%-45% of the total volume. In some instances, hybridization buffers described herein are used in conjunction with universal blockers and liquid polymer additives.

Hybridization buffers described herein may comprise salts such as cations or anions. For example, hybridization buffer comprises a monovalent or divalent cation. In some instances, a hybridization buffer comprises a monovalent or divalent anion. Cations in some instances comprise sodium, potassium, magnesium, lithium, tris, or other salt. Anions in some instances comprise sulfate, bisulfite, hydrogensulfate, nitrate, chloride, bromide, citrate, ethylenediaminetetraacetate, dihydrogenphosphate, hydrogenphosphate, or phosphate. In some instances, hybridization buffers comprise salts comprising any combination of anions and cations (e.g. sodium chloride, sodium sulfate, potassium phosphate, or other salt). In some instance, a hybridization buffer comprises an ionic liquid. Salts described herein may be present at any concentration suitable for reducing off-target binding. Such concentrations are often represented as a percent by weight, percent by volume, or percent weight per volume. For example, a salt is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a salt is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a salt is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a salt is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a salt is present at 0.005%-0.1%. In some instances, a salt is present at 0.05%-0.1%. In some instances, a salt is present at 0.005%-0.6%. In some instances, a salt is present at 1%-30%, 5%-25%, 10%-30%, 15%-30%, or 1%-15%. Liquid polymers may be present as a percentage of the total reaction volume. In some instances, a salt is about 10%, 20%, 30%, 40%, 50%, 60%, 75%, or about 90% of the total volume. In some instances, a salt is at least 10%, 20%, 30%, 40%, 50%, 60%, 75%, or at least 90% of the total volume. In some instances, a salt is no more than 10%, 20%, 30%, 40%, 50%, 60%, 75%, or no more than 90% of the total volume. In some instances, a salt is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a salt is 25%-45% of the total volume.

Hybridization buffers described herein may comprise surfactants (or emulsifiers). For example, a hybridization buffer comprises SDS (sodium dodecyl sulfate), CTAB, cetylpyridinium, benzalkonium tergitol, fatty acid sulfonates (e.g., sodium lauryl sulfate), ethyloxylated propylene glycol, lignin sulfonates, benzene sulfonate, lecithin, phospholipids, dialkyl sulfosuccinates (e.g., dioctyl sodium sulfosuccinate), glycerol diester, polyethoxylated octyl phenol, abietic acid, sorbitan monoester, perfluoro alkanols, sulfonated polystyrene, betaines, dimethyl polysiloxanes, or other surfactant. In some instances, a hybridization buffer comprises a sulfate, phosphate, or tetralkyl ammonium group. Surfactants described herein may be present at any concentration suitable for reducing off-target binding. Such concentrations are often represented as a percent by weight, percent by volume, or percent weight per volume. For example, a surfactant is present at about 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or about 30%. In some instances, a surfactant is present at no more than 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or no more than 30%. In some instances, a surfactant is present in at least 0.0001%, 0.0002%, 0.0005%, 0.0008%, 0.001%, 0.002%, 0.005%, 0.008%, 0.01%, 0.02%, 0.05%, 0.08%, 0.1%, 0.2%, 0.5%, 0.8%, 1%, 1.2%, 1.5%, 1.8%, 2%, 5%, 10%, 20%, or at least 30%. In some instances, a surfactant is present at 0.0001%-10%, 0.0002%-5%, 0.0005%-1.5%, 0.0008%-1%, 0.001%-0.2%, 0.002%-0.08%, 0.005%-0.02%, or 0.008%-0.05%. In some instances, a surfactant is present at 0.005%-0.1%. In some instances, a surfactant is present at 0.05%-0.1%. In some instances, a surfactant is present at 0.005%-0.6%. In some instances, a surfactant is present at 1%-30%, 5%-25%, 10%-30%, 15%-30%, or 1%-15%. Liquid polymers may be present as a percentage of the total reaction volume. In some instances, a surfactant is about 10%, 20%, 30%, 40%, 50%, 60%, 75%, or about 90% of the total volume. In some instances, a surfactant is at least 10%, 20%, 30%, 40%, 50%, 60%, 75%, or at least 90% of the total volume. In some instances, a surfactant is no more than 10%, 20%, 30%, 40%, 50%, 60%, 75%, or no more than 90% of the total volume. In some instances, a surfactant is 5%-75%, 5%-65%, 5%-55%, 10%-50%, 15%-40%, 20%-50%, 20%-30%, 25%-35%, 5%-35%, 10%-35%, or 20%-40% of the total volume. In some instances, a surfactant is 25%-45% of the total volume.

Buffers used in the methods described herein may comprise any combination of components. In some instances, a buffer described herein is a hybridization buffer. In some instances, a hybridization buffer described herein is a fast hybridization buffer. Such fast hybridization buffers allow for lower hybridization times such as less than 8 hours, 6 hours, 4 hours, 2 hours, 1 hour, 45 minutes, 30 minutes, or less than 15 minutes. Hybridization buffers described herein in some instances comprise a buffer described in Tables 2A-2G. In some instances, the buffers described in Tables 1A-1I may be used as fast hybridization buffers. In some instances, the buffers described in Tables 1B, 1C, and 1D may be used as fast hybridization buffers. In some instances, a fast hybridization buffer as described herein is described in Table 1B. In some instances, a fast hybridization buffer as described herein is described in Table 1C. In some instances, a fast hybridization buffer as described herein is described in Table 1D.

TABLE 2A
Buffers A

| Buffer | Volume | Buffer | Volume |
|---|---|---|---|
| Component | (mL) | Component | (mL) |
| Water | 5-300 | Water | 100-300 |
| DMF | 0-3 | DMSO | 0-3 |
| NaCl (5M) | 0.01-0.5 | NaCl (5M) | 0.01-0.5 |
| 20% SDS | 0.05-0.5 | 20% SDS | 0.05-0.5 |
| Tergitol | 0.2-3 | EDTA (1M) | 0-2 |
| (1% by weight) | | | |
| Denhardt's | 1-10 | Denhardt's | 1-10 |
| Solution (50X) | | Solution (50X) | |
| NaH₂PO₄ (5M) | 0.01-1.5 | NaH₂PO₄ (5M) | 0.01-1.5 |

TABLE 2B
Buffers B

| Buffer | Volume | Buffer | Volume |
|---|---|---|---|
| Component | (mL) | Component | (mL) |
| Water | 5-30 | Water | 5-30 |
| DMSO | 0.5-3 | DMSO | 0.5-3 |
| NaCl (5M) | 0.01-0.5 | NaCl (5M) | 0.01-0.5 |
| 20% SDS | 0.05-0.5 | 20% CTAB | 0.05-0.5 |
| EDTA (1M) | 0.05-2 | EDTA (1M) | 0.05-2 |
| Denhardt's | 1-10 | Denhardt's | 1-10 |
| Solution (50X) | | Solution (50X) | |
| NaH₂PO₄ (5M) | 0.01-1.5 | NaH₂PO₄ (5M) | 0.01-1.5 |

TABLE 2C
Buffers C

| Buffer | Volume | Buffer | Volume |
|---|---|---|---|
| Component | (mL) | Component | (mL) |
| Water | 5-30 | Water | 5-30 |
| DMSO | 0.5-3 | DMSO | 0.5-3 |
| NaCl (1M) | 0.01-0.5 | NaCl (5M) | 0.01-0.5 |
| 20% SDS | 0.05-0.5 | 20% SDS | 0.05-0.5 |
| TrisHCl (1M) | 0.01-2.5 | Dextran Sulfate (50%) | 0.05-2 |
| Denhardt's | 1-10 | Denhardt's | 1-10 |
| Solution (50X) | | Solution (50X) | |
| NaH₂PO₄ (5M) | 0.01-1.5 | NaH₂PO₄ (5M) | 0.01-1.5 |
| EDTA (0.5M) | 0.05-1.5 | EDTA (0.5M) | 0.05-1.5 |

TABLE 2D
Buffers D

| Buffer | Volume | Buffer | Volume |
|---|---|---|---|
| Component | (mL) | Component | (mL) |
| Water | 5-30 | Water | 5-30 |
| Methanol | 0.1-3 | DMSO | 0.5-3 |
| NaCl (1M) | 0.01-0.5 | NaCl (5M) | 0.01-0.5 |
| 20% Dextran Sulfate | 0.05-0.5 | 20% SDS | 0.05-0.5 |
| TrisHCl (1M) | 0.01-2.5 | hydroxyethyl | 0.05-2 |
| | | starch (20%) | |
| Denhardt's | 1-10 | Denhardt's | 1-10 |
| Solution (50X) | | Solution (50X) | |
| NaH₂PO₄ (1M) | 0.01-1.5 | NaH₂PO₄ (5M) | 0.01-1.5 |
| EDTA (0.5M) | 0.05-1.5 | EDTA (0.5M) | 0.05-1.5 |

TABLE 2E
Buffers E

| Buffer | Volume | Buffer | Volume |
|---|---|---|---|
| Component | (mL) | Component | (mL) |
| Water | 5-300 | Water | 5-300 |
| DMF | 0.1-30 | DMSO | 0.5-30 |
| NaCl (1M) | 0.01-0.5 | NaCl (5M) | 0.01-1.0 |
| hydroxyethyl | 0.01-2.5 | hydroxyethyl | 0.01-2.5 |
| starch (20%) | | starch (20%) | |
| Denhardt's | 1-10 | Denhardt's | 0.05-2 |
| Solution (50X) | | Solution (50X) | |
| NaH₂PO₄ (1M) | 0.01-1.5 | NaH₂PO₄ (5M) | 1-10 |

TABLE 2F
Buffers F

| Buffer | Volume | Buffer | Volume |
|---|---|---|---|
| Component | (mL) | Component | (mL) |
| Water | 50-300 | Water | 50-300 |
| DMF | 15-300 | DMSO | 15-300 |
| NaCl (5M) | 2-100 | NaCl (5M) | 2-100 |
| Denhardt's | 1-10 | saline-sodium | 1-50 |
| Solution (50X) | | citrate 20X | |
| Tergitol | 0.2-2.0 | 20% SDS | 0-2 |
| (1% by weight) | | | |

TABLE 2G
Buffers G

| Buffer | Volume | Buffer | Volume |
|---|---|---|---|
| Component | (mL) | Component | (mL) |
| Water | 5-30 | Water | 5-30 |
| Ethanol | 0-3 | Methanol | 0-3 |
| NaCl (1M) | 0.01-0.5 | NaCl (5M) | 0.01-0.5 |
| NaH₂PO₄ (5M) | 0.01-1.5 | NaH₂PO₄ (5M) | 0-2 |
| EDTA (0.5M) | 0-1.5 | EDTA (0.5M) | 1-10 |

TABLE 2H
Buffers H

| Buffer | Volume | Buffer | Volume |
|---|---|---|---|
| Component | (mL) | Component | (mL) |
| Water | 50-300 | Water | 10-300 |
| EDTA (0.5M) | 0-1.5 | NaCl (5M) | 0.01-0.5 |
| NaCl (5M) | 5-70 | 10% Triton X-100 | 0.05-0.5 |
| Tergitol | 0.2-2.0 | EDTA (1M) | 0-2 |
| (1% by weight) | | | |
| TrisHCl (1M) | 0.01-2.5 | TrisHCl (1M) | 0.1-5 |

TABLE 2I
Buffers I

| Buffer | Volume | Buffer | Volume |
|---|---|---|---|
| Component | (mL) | Component | (mL) |
| Water | 5-200 | Water | 10-200 |
| EDTA (0.5M) | 0-1.5 | NaCl (5M) | 0.01-0.5 |
| NaCl (5M) | 5-100 | Sodium Lauryl | 0.05-0.5 |
| | | sulfate (10%) | |
| CTAB (0.2M) | 0.05-0.5 | EDTA (1M) | 0-2 |

Buffers such as binding buffers and wash buffers are described herein. Binding buffers in some instances are used to prepare mixtures of sample polynucleotides and probes after hybridization. In some instances, binding buffers facilitate capture of sample polynucleotides on a column or other solid support. In some instances, the buffers described in Tables 2A-2I may be used as binding buffers. Binding buffers in some instances comprise a buffer described in Tables 2A, 2H, and 2I. In some instances, a binding buffer as described herein is described in Table 2A. In some instances, a binding buffer as described herein is described in Table 2H. In some instances, a binding buffer as described herein is described in Table 21. In some instances, the buffers described herein may be used as wash buffers. Wash buffers in some instances are used to remove non-binding polynucleotides from a column or solid support. In some instances, the buffers described in Tables 2A-2I may be used as wash buffers. In some instances, a wash buffer comprises a buffer as described in Tables 2E, 2F, and 2G. In some instances, a wash buffer as described herein is described in Table 2E. In some instances, a wash buffer as described herein is described in Table 2F. In some instances, a wash buffer as described herein is described in Table 2G. Wash buffers used with the compositions and methods described herein are in some instances described as a first wash buffer (wash buffer 1), second wash buffer (wash buffer 2), etc.

### Methods for Sequencing

Described herein are methods to improve the efficiency and accuracy of sequencing. Such methods comprise use of universal adapters comprising nucleobase analogues, and generation of barcoded adapters after ligation to sample nucleic acids. In some instances, a sample is fragmented, fragment ends are repaired, one or more adenines is added to one strand of a fragment duplex, universal adapters are ligated, and a library of fragments is amplified with barcoded primers to generate a barcoded nucleic acid library. Additional steps in some instances include enrichment/capture, additional PCR amplification, and/or sequencing of the nucleic acid library.

Sequencing may be used to analyze methylation patterns. In some instances, methods provided herein acquire sufficient reads to achieve at least 1, 2, 5, 10, 15, 20, 30, or at least 50× coverage target regions. In some instances, target regions comprise at least 0.1, 0.5, 1, 1.5, 2, 2.5, 3, 4, 5, 6, or at least 7 million CpG sites. In some instances, sequencing further comprises molecular counting using UMIs.

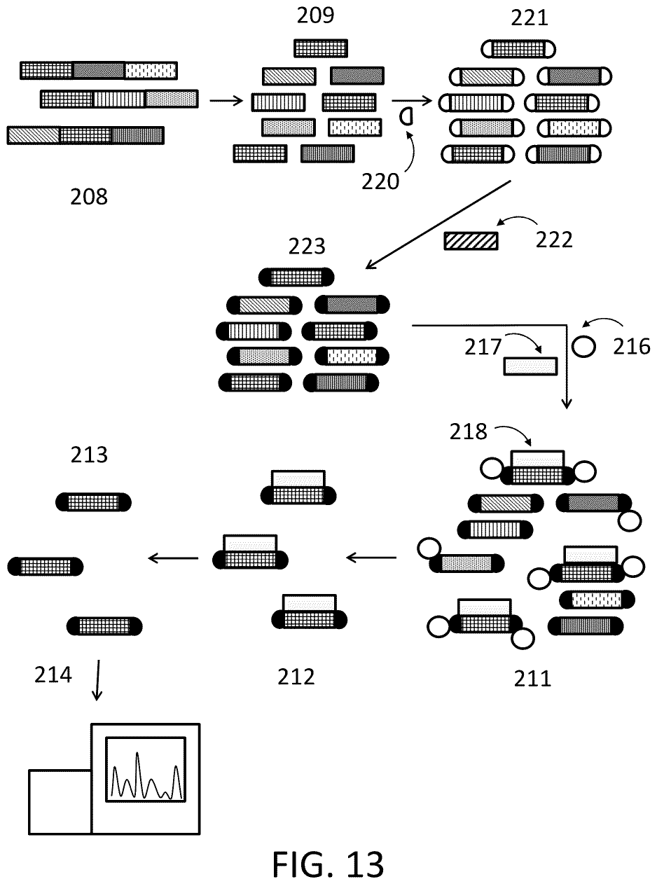

In a first step of an exemplary sequencing workflow (FIG. 13), a sample 208 comprising sample nucleic acids is fragmented by mechanical or enzymatic shearing to form a library of fragments 209. Universal adapters 220 are ligated to fragmented sample nucleic acids to form an adapter-ligated sample nucleic acid library 221. This library is then amplified with a barcoded primer library 222 (only one primer shown for simplicity) to generate a barcoded adapter-sample polynucleotide library 223. The library 223 is then optionally hybridized with target binding polynucleotides 217, which hybridize to sample nucleic acids, along with blocking polynucleotides 216 that prevent hybridization between probe polynucleotides 217 and adapters 220. Capture of sample polynucleotide-target binding polynucleotide hybridization pairs 212/218, and removal of target binding polynucleotides 217 allows isolation/enrichment of sample nucleic acids 213, which are then optionally amplified and sequenced 214. Various combinations of universal adapters and barcoded primers may be used. In some instances, barcoded primers comprise at least one barcode. In some instances, different types of barcodes are added to the sample nucleic acid using adapters or barcodes, or both. For example, a universal adapter comprises an index barcode, and after ligation is amplified with a barcoded primer comprising an additional index barcode. In some instances, a universal adapter comprises a unique molecular identifier barcode, and after ligation is amplified with a barcoded primer comprising an index barcode.

Barcoded primers may be used to amplify universal adapter-ligated sample polynucleotides using PCR, to generate a polynucleic acid library for sequencing. Such a library comprises barcodes after amplification in some instances. In some instances, amplification with barcoded primers results in higher amplification yields relative to amplification of a standard Y adapter-ligated sample polynucleotide library. In some instances, 2, 3, 4, 5, 6, 7, 8, 9, 10, 11, or 12 PCR cycles are used to amplify a universal adapter-ligated sample polynucleotide library. In some instances, no more than 2, 3, 4, 5, 6, 7, 8, 9, 10, 11, or no more than 12 PCR cycles are used to amplify a universal adapter-ligated sample polynucleotide library. In some instances, 2-12, 3-10, 4-9, 5-8, 6-10, or 8-12 PCR cycles are used to amplify a universal adapter-ligated sample polynucleotide library, thus generating amplicon products. Such libraries in some instances comprise fewer PCR-based errors. Without being bound by theory, reduced PCR cycles during amplification leads to fewer errors in resulting amplicon products. After amplification, such barcoded amplicon libraries are in some instances enriched or subjected to capture, additional amplification reactions, and/or sequencing. In some instances, amplicon products generated using the universal adapters described herein comprise about 30%, 15%, 10%, 7%, 5%, 3%, 2%, 1.5%, 1%, 0.5%, 0.1%, or 0.05% fewer errors than amplicon products generated from amplification of standard full-length Y adapters.

Described herein are methods wherein universal blockers are used to prevent off-target binding of capture probes to adapters ligated to genomic fragments, or adapter-adapter hybridization. Adapter blockers used for preventing off-target hybridization may target a portion or the entire adapter. In some instances, specific blockers are used that are complementary to a portion of the adapter that includes the unique index sequence. In cases where the adapter-tagged genomic library comprises a large number of different indices, it can be beneficial to design blockers which either do not target the index sequence, or do not hybridize strongly to it. For example, a “universal” blocker targets a portion of the adapter that does not comprise an index sequence (index independent), which allows a minimum number of blockers to be used regardless of the number of different index sequences employed. In some instances, no more than 8 universal blockers are used. In some instances, 4 universal blockers are used. In some instances, 3 universal blockers are used. In some instances, 2 universal blockers are used. In some instances, 1 universal blocker is used. In an exemplary arrangement, 4 universal blockers are used with adapters comprising at least 4, 8, 16, 32, 64, 96, or at least 128 different index sequences. In some instances, the different index sequences comprises at least or about 4, 6, 8, 10, 12, 14, 16, 18, 20, or more than 20 base pairs (bp). In some instances, a universal blocker is not configured to bind to a barcode sequence. In some instances, a universal blocker partially binds to a barcode sequence. In some instances, a universal blocker which partially binds to a barcode sequence further comprises nucleotide analogs, such as those that increase the Tm of binding to the adapter (e.g., LNAs or BNAs).

### Methylation Sequencing and Capture

Methylation sequencing involves enzymatic or chemical methods leading to the conversion of unmethylated cytosines to uracil through a series of events culminating in deamination, while leaving methylated cytosines intact. During amplification, uracils are paired with adenines on the complementary strand, leading to the inclusion of thymine in the original position of the unmethylated cytosine. There are identical sequences with each having unmethylated-cytosines in different positions. The end product is asymmetric, yielding two different double stranded DNA molecules after conversion; the same process for methylated DNA leads to yet additional sets of sequences.

Target enrichment can proceed by pre- or post-capture conversion. Post-capture conversion targets the original sample DNA, while pre-capture targets the four strands of converted sequences. While post-capture conversion presents fewer challenges for probe design, it often requires large quantities of starting DNA material as PCR amplification does not preserve methylation patterns and cannot be performed before capture. Therefore, pre-capture conversion is often the method of choice for low-input, sensitive applications such as cell free DNA.

Methods described herein may comprise treatment of a library with enzymes or bisulfite to facilitate conversion of cytosines to uracil. In some instances, adapters (e.g., universal adapters) described herein comprise methylated nucleobases, such as methylated cytosine.

Methods of measuring methylation may comprise use of hybridization reagents described herein. Provided herein are methods comprising one or more steps of: providing a plurality of sequences encoding one or more source polynucleotides derived from an organism, wherein the source polynucleotides comprise a C0t value; mapping the plurality of sequences onto a bisulfite or enzymatic deamination-treated reference genome to generate mapped sequences; and synthesizing a hybridization reagent, wherein the hybridization reagent comprises a plurality of modified polynucleotides comprising mapped sequences of the reference genome. In some instances, the method further comprises removal of mapped sequences comprising exome and refseq sequences prior synthesizing the hybridization reagent. Provided herein are methods comprising one or more of providing a plurality of sequences encoding one or more source polynucleotides derived from an organism, wherein the source polynucleotides comprise a Cot value; modifying the plurality of sequences, wherein modifying comprises replacement of at least one cytosine with uracil or thymine in the plurality of sequences to generate a plurality of modified sequences; and synthesizing a hybridization reagent, wherein the hybridization reagent comprises a plurality of modified polynucleotides comprising the plurality of modified sequences. In some instances the Cot value is no more than 5, 4, 3, 2.5, 2.25, 2, 1.75, 1.50 1.25, 1, or no more than 0.75. In some instances the Cot value is 0.01-4, 0.01-3, 0.01-2, 0.01-1.5, 0.1-3, 0.1-2.5, 0.1-2, 0.1-1.7, 0.1-1.5, or 0.1-1.25.

### De Novo Synthesis of Small Polynucleotide Populations for Amplification Reactions

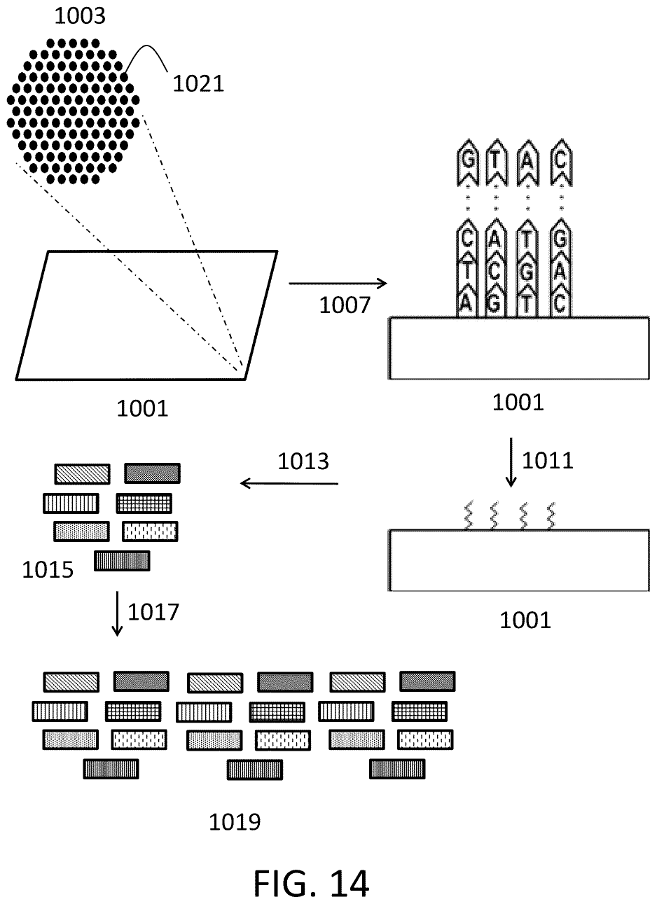

Described herein are methods of synthesis of polynucleotides from a surface, e.g., a plate (FIG. 14). In some instances, the polynucleotides are synthesized on a cluster of loci for polynucleotide extension, released and then subsequently subjected to an amplification reaction, e.g., PCR. An exemplary workflow of synthesis of polynucleotides from a cluster is depicted in FIG. 14. A silicon plate 1001 includes multiple clusters 1003. Within each cluster are multiple loci 1021. Polynucleotides are synthesized 1007 de novo on a plate 1001 from the cluster 1003. Polynucleotides are cleaved 1011 and removed 1013 from the plate to form a population of released polynucleotides 1015. The population of released polynucleotides 1015 is then amplified 1017 to form a library of amplified polynucleotides 1019.

Provided herein are methods where amplification of polynucleotides synthesized on a cluster provide for enhanced control over polynucleotide representation compared to amplification of polynucleotides across an entire surface of a structure without such a clustered arrangement. In some instances, amplification of polynucleotides synthesized from a surface having a clustered arrangement of loci for polynucleotides extension provides for overcoming the negative effects on representation due to repeated synthesis of large polynucleotide populations. Exemplary negative effects on representation due to repeated synthesis of large polynucleotide populations include, without limitation, amplification bias resulting from high/low GC content, repeating sequences, trailing adenines, secondary structure, affinity for target sequence binding, or modified nucleotides in the polynucleotide sequence.

Cluster amplification as opposed to amplification of polynucleotides across an entire plate without a clustered arrangement can result in a tighter distribution around the mean. For example, if 100,000 reads are randomly sampled, an average of 8 reads per sequence would yield a library with a distribution of about 1.5× from the mean. In some cases, single cluster amplification results in at most about 1.5×, 1.6×, 1.7×, 1.8×, 1.9×, or 2.0× from the mean. In some cases, single cluster amplification results in at least about 1.0×, 1.2×, 1.3×, 1.5×1.6×, 1.7×, 1.8×, 1.9×, or 2.0× from the mean.

Cluster amplification methods described herein when compared to amplification across a plate can result in a polynucleotide library that requires less sequencing for equivalent sequence representation. In some instances at least 10%, at least 20%, at least 30%, at least 40%, at least 50%, at least 60%, at least 70%, at least 80%, at least 90%, or at least 95% less sequencing is required. In some instances up to 10%, up to 20%, up to 30%, up to 40%, up to 50%, up to 60%, up to 70%, up to 80%, up to 90%, or up to 95% less sequencing is required. Sometimes 30% less sequencing is required following cluster amplification compared to amplification across a plate. Sequencing of polynucleotides in some instances is verified by high-throughput sequencing such as by next generation sequencing. Sequencing of the sequencing library can be performed with any appropriate sequencing technology, including but not limited to single-molecule real-time (SMRT) sequencing, polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. The number of times a single nucleotide or polynucleotide is identified or “read” is defined as the sequencing depth or read depth. In some cases, the read depth is referred to as a fold coverage, for example, 55 fold (or 55×) coverage, optionally describing a percentage of bases.

In some instances, amplification from a clustered arrangement compared to amplification across a plate results in less dropouts, or sequences which are not detected after sequencing of amplification product. Dropouts can be of AT and/or GC. In some instances, a number of dropouts are at most about 1%, 2%, 3%, 4%, or 5% of a polynucleotide population. In some cases, the number of dropouts is zero.

A cluster as described herein comprises a collection of discrete, non-overlapping loci for polynucleotide synthesis. A cluster can comprise about 50-1000, 75-900, 100-800, 125-700, 150-600, 200-500, or 300-400 loci. In some instances, each cluster includes 121 loci. In some instances, each cluster includes about 50-500, 50-200, 100-150 loci. In some instances, each cluster includes at least about 50, 100, 150, 200, 500, 1000 or more loci. In some instances, a single plate includes 100, 500, 10000, 20000, 30000, 50000, 100000, 500000, 700000, 1000000 or more loci. A locus can be a spot, well, microwell, channel, or post. In some instances, each cluster has at least 1×, 2×, 3×, 4×, 5×, 6×, 7×, 8×, 9×, 10×, or more redundancy of separate features supporting extension of polynucleotides having identical sequence.

Generation of Polynucleotide Libraries with Controlled Stoichiometry of Sequence Content

In some instances, the polynucleotide library is synthesized with a specified distribution of desired polynucleotide sequences. In some instances, adjusting polynucleotide libraries for enrichment of specific desired sequences results in improved downstream application outcomes.

One or more specific sequences can be selected based on their evaluation in a downstream application. In some instances, the evaluation is binding affinity to target sequences for amplification, enrichment, or detection, stability, melting temperature, biological activity, ability to assemble into larger fragments, or other property of polynucleotides. In some instances, the evaluation is empirical or predicted from prior experiments and/or computer algorithms. An exemplary application includes increasing sequences in a probe library which correspond to areas of a genomic target having less than average read depth.

Selected sequences in a polynucleotide library can be at least 10%, 20%, 30%, 40%, 50%, 60%, 70%, 80%, 90%, 95%, or more than 95% of the sequences. In some instances, selected sequences in a polynucleotide library are at most 10%, 20%, 30%, 40%, 50%, 60%, 70%, 80%, 90%, 95%, or at most 100% of the sequences. In some cases, selected sequences are in a range of about 5-95%, 10-90%, 30-80%, 40-75%, or 50-70% of the sequences.

Polynucleotide libraries can be adjusted for the frequency of each selected sequence. In some instances, polynucleotide libraries favor a higher number of selected sequences. For example, a library is designed where increased polynucleotide frequency of selected sequences is in a range of about 40% to about 90%. In some instances, polynucleotide libraries contain a low number of selected sequences. For example, a library is designed where increased polynucleotide frequency of the selected sequences is in a range of about 10% to about 60%. A library can be designed to favor a higher and lower frequency of selected sequences. In some instances, a library favors uniform sequence representation. For example, polynucleotide frequency is uniform with regard to selected sequence frequency, in a range of about 10% to about 90%. In some instances, a library comprises polynucleotides with a selected sequence frequency of about 10% to about 95% of the sequences.

Generation of polynucleotide libraries with a specified selected sequence frequency in some cases occurs by combining at least 2 polynucleotide libraries with different selected sequence frequency content. In some instances, at least 2, 3, 4, 5, 6, 7, 10, or more than 10 polynucleotide libraries are combined to generate a population of polynucleotides with a specified selected sequence frequency. In some cases, no more than 2, 3, 4, 5, 6, 7, or 10 polynucleotide libraries are combined to generate a population of non-identical polynucleotides with a specified selected sequence frequency.

In some instances, selected sequence frequency is adjusted by synthesizing fewer or more polynucleotides per cluster. For example, at least 25, 50, 100, 200, 300, 400, 500, 600, 700, 800, 900, 1000, or more than 1000 non-identical polynucleotides are synthesized on a single cluster. In some cases, no more than about 50, 100, 200, 300, 400, 500, 600, 700, 800, 900, 1000 non-identical polynucleotides are synthesized on a single cluster. In some instances, 50 to 500 non-identical polynucleotides are synthesized on a single cluster. In some instances, 100 to 200 non-identical polynucleotides are synthesized on a single cluster. In some instances, about 100, about 120, about 125, about 130, about 150, about 175, or about 200 non-identical polynucleotides are synthesized on a single cluster.

In some cases, selected sequence frequency is adjusted by synthesizing non-identical polynucleotides of varying length. For example, the length of each of the non-identical polynucleotides synthesized may be at least or about at least 10, 15, 20, 25, 30, 35, 40, 45, 50, 100, 150, 200, 300, 400, 500, 2000 nucleotides, or more. The length of the non-identical polynucleotides synthesized may be at most or about at most 2000, 500, 400, 300, 200, 150, 100, 50, 45, 35, 30, 25, 20, 19, 18, 17, 16, 15, 14, 13, 12, 11, 10 nucleotides, or less. The length of each of the non-identical polynucleotides synthesized may fall from 10-2000, 10-500, 9-400, 11-300, 12-200, 13-150, 14-100, 15-50, 16-45, 17-40, 18-35, and 19-25.

### Polynucleotide Probe Structures

Libraries of polynucleotide probes can be used to enrich particular target sequences in a larger population of sample polynucleotides. In some instances, polynucleotide probes each comprise a target binding sequence complementary to one or more target sequences, one or more non-target binding sequences, and one or more primer binding sites, such as universal primer binding sites. Target binding sequences that are complementary or at least partially complementary in some instances bind (hybridize) to target sequences. Primer binding sites, such as universal primer binding sites facilitate simultaneous amplification of all members of the probe library, or a subpopulation of members. In some instances, the probes or adapters further comprise a barcode or index sequence. Barcodes are nucleic acid sequences that allow some feature of a polynucleotide with which the barcode is associated to be identified. After sequencing, the barcode region provides an indicator for identifying a characteristic associated with the coding region or sample source. Barcodes can be designed at suitable lengths to allow sufficient degree of identification, e.g., at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or more bases in length. Multiple barcodes, such as about 2, 3, 4, 5, 6, 7, 8, 9, 10, or more barcodes, may be used on the same molecule, optionally separated by non-barcode sequences. In some instances, each barcode in a plurality of barcodes differ from every other barcode in the plurality at least three base positions, such as at least about 3, 4, 5, 6, 7, 8, 9, 10, or more positions. Use of barcodes allows for the pooling and simultaneous processing of multiple libraries for downstream applications, such as sequencing (multiplex). In some instances, at least 4, 8, 16, 32, 48, 64, 128, 512, 1024, 2000, 5000, or more than 5000 barcoded libraries are used. In some instances, the polynucleotides are ligated to one or more molecular (or affinity) tags such as a small molecule, peptide, antigen, metal, or protein to form a probe for subsequent capture of the target sequences of interest. In some instances, only a portion of the polynucleotides are ligated to a molecular tag. In some instances, two probes that possess complementary target binding sequences which are capable of hybridization form a double stranded probe pair. Polynucleotide probes or adapters may comprise unique molecular identifiers (UMI). UMIs allow for internal measurement of initial sample concentrations or stoichiometry prior to downstream sample processing (e.g., PCR or enrichment steps) which can introduce bias. In some instances, UMIs comprise one or more barcode sequences.

Probes described here may be complementary to target sequences which are sequences in a genome. Probes described here may be complementary to target sequences which are exome sequences in a genome. Probes described here may be complementary to target sequences which are intron sequences in a genome. In some instances, probes comprise a target binding sequence complementary to a target sequence (of the sample nucleic acid), and at least one non-target binding sequence that is not complementary to the target. In some instances, the target binding sequence of the probe is about 120 nucleotides in length, or at least 10, 15, 20, 25, 50, 75, 100, 110, 120, 125, 140, 150, 160, 175, 200, 300, 400, 500, or more than 500 nucleotides in length. The target binding sequence is in some instances no more than 10, 15, 20, 25, 50, 75, 100, 125, 150, 175, 200, or no more than 500 nucleotides in length. The target binding sequence of the probe is in some instances about 120 nucleotides in length, or about 10, 15, 20, 25, 40, 50, 60, 70, 80, 85, 87, 90, 95, 97, 100, 105, 110, 115, 117, 118, 119, 120, 121, 122, 123, 124, 125, 126, 127, 128, 129, 130, 135, 140, 145, 150, 155, 157, 158, 159, 160, 161, 162, 163, 164, 165, 166, 167, 168, 169, 170, 175, 180, 190, 200, 210, 220, 230, 240, 250, 300, 400, or about 500 nucleotides in length. The target binding sequence is in some instances about 20 to about 400 nucleotides in length, or about 30 to about 175, about 40 to about 160, about 50 to about 150, about 75 to about 130, about 90 to about 120, or about 100 to about 140 nucleotides in length. The non-target binding sequence(s) of the probe is in some instances at least about 20 nucleotides in length, or at least about 1, 5, 10, 15, 17, 20, 23, 25, 50, 75, 100, 110, 120, 125, 140, 150, 160, 175, or more than about 175 nucleotides in length. The non-target binding sequence often is no more than about 5, 10, 15, 20, 25, 50, 75, 100, 125, 150, 175, or no more than about 200 nucleotides in length. The non-target binding sequence of the probe often is about 20 nucleotides in length, or about 1, 2, 3, 4, 5, 6, 7, 8, 9, 10, 11, 12, 13, 14, 15, 16, 17, 18, 19, 20, 21, 22, 23, 25, 40, 50, 60, 70, 80, 90, 100, 110, 120, 130, 140, 150, or about 200 nucleotides in length. The non-target binding sequence in some instances is about 1 to about 250 nucleotides in length, or about 20 to about 200, about 10 to about 100, about 10 to about 50, about 30 to about 100, about 5 to about 40, or about 15 to about 35 nucleotides in length. The non-target binding sequence often comprises sequences that are not complementary to the target sequence, and/or comprise sequences that are not used to bind primers. In some instances, the non-target binding sequence comprises a repeat of a single nucleotide, for example polyadenine or polythymidine. A probe often comprises none or at least one non-target binding sequence. In some instances, a probe comprises one or two non-target binding sequences. The non-target binding sequence may be adjacent to one or more target binding sequences in a probe. For example, a non-target binding sequence is located on the 5′ or 3′ end of the probe. In some instances, the non-target binding sequence is attached to a molecular tag or spacer.

In some instances, the non-target binding sequence(s) may be a primer binding site. The primer binding sites often are each at least about 20 nucleotides in length, or at least about 10, 12, 14, 16, 18, 20, 22, 24, 26, 28, 30, 32, 34, 36, 38, or at least about 40 nucleotides in length. Each primer binding site in some instances is no more than about 10, 12, 14, 16, 18, 20, 22, 24, 26, 28, 30, 32, 34, 36, 38, or no more than about 40 nucleotides in length. Each primer binding site in some instances is about 10 to about 50 nucleotides in length, or about 15 to about 40, about 20 to about 30, about 10 to about 40, about 10 to about 30, about 30 to about 50, or about 20 to about 60 nucleotides in length. In some instances the polynucleotide probes comprise at least two primer binding sites. In some instances, primer binding sites may be universal primer binding sites, wherein all probes comprise identical primer binding sequences at these sites. In some instances, a pair of polynucleotide probes targeting a particular sequence and its reverse complement (e.g., a region of genomic DNA), comprising a first target binding sequence, a second target binding sequence, a first non-target binding sequence, and a second non-target binding sequence. For example, a pair of polynucleotide probes complementary to a particular sequence (e.g., a region of genomic DNA).

In some instances, the first target binding sequence is the reverse complement of the second target binding sequence. In some instances, both target binding sequences are chemically synthesized prior to amplification. In an alternative arrangement, a pair of polynucleotide probes targeting a particular sequence and its reverse complement (e.g., a region of genomic DNA) comprise a first target binding sequence, a second target binding sequence, a first non-target binding sequence, a second non-target binding sequence, a third non-target binding sequence, and a fourth non-target binding sequence. In some instances, the first target binding sequence is the reverse complement of the second target binding sequence. In some instances, one or more non-target binding sequences comprise polyadenine or polythymidine.

In some instances, both probes in the pair are labeled with at least one molecular tag. In some instances, PCR is used to introduce molecular tags (via primers comprising the molecular tag) onto the probes during amplification. In some instances, the molecular tag comprises one or more biotin, folate, a polyhistidine, a FLAG tag, glutathione, or other molecular tag consistent with the specification. In some instances probes are labeled at the 5′ terminus. In some instances, the probes are labeled at the 3′ terminus. In some instances, both the 5′ and 3′ termini are labeled with a molecular tag. In some instances, the 5′ terminus of a first probe in a pair is labeled with at least one molecular tag, and the 3′ terminus of a second probe in the pair is labeled with at least one molecular tag. In some instances, a spacer is present between one or more molecular tags and the nucleic acids of the probe. In some instances, the spacer may comprise an alkyl, polyol, or polyamino chain, a peptide, or a polynucleotide. The solid support used to capture probe-target nucleic acid complexes in some instances, is a bead or a surface. The solid support in some instances comprises glass, plastic, or other material capable of comprising a capture moiety that will bind the molecular tag. In some instances, a bead is a magnetic bead. For example, probes labeled with biotin are captured with a magnetic bead comprising streptavidin. The probes are contacted with a library of nucleic acids to allow binding of the probes to target sequences. In some instances, blocking polynucleic acids are added to prevent binding of the probes to one or more adapter sequences attached to the target nucleic acids. In some instances, blocking polynucleic acids comprise one or more nucleic acid analogues. In some instances, blocking polynucleic acids have a uracil substituted for thymine at one or more positions.

Probes described herein may comprise complementary target binding sequences which bind to one or more target nucleic acid sequences. In some instances, the target sequences are any DNA or RNA nucleic acid sequence. In some instances, target sequences may be longer than the probe insert. In some instance, target sequences may be shorter than the probe insert. In some instance, target sequences may be the same length as the probe insert. For example, the length of the target sequence may be at least or about at least 2, 10, 15, 20, 25, 30, 35, 40, 45, 50, 100, 150, 200, 300, 400, 500, 1000, 2000, 5,000, 12,000, 20,000 nucleotides, or more. The length of the target sequence may be at most or about at most 20,000, 12,000, 5,000, 2,000, 1,000, 500, 400, 300, 200, 150, 100, 50, 45, 35, 30, 25, 20, 19, 18, 17, 16, 15, 14, 13, 12, 11, 10, 2 nucleotides, or less. The length of the target sequence may fall from 2-20,000, 3-12,000, 5-5, 5000, 10-2,000, 10-1,000, 10-500, 9-400, 11-300, 12-200, 13-150, 14-100, 15-50, 16-45, 17-40, 18-35, and 19-25. The probe sequences may target sequences associated with specific genes, diseases, regulatory pathways, or other biological functions consistent with the specification.

In some instances, a single probe insert is complementary to one or more target sequences in a larger polynucleic acid (e.g., sample nucleic acid). An exemplary target sequence is an exon. In some instances, one or more probes target a single target sequence. In some instances, a single probe may target more than one target sequence. In some instances, the target binding sequence of the probe targets both a target sequence and an adjacent sequence. In some instances, a first probe targets a first region and a second region of a target sequence, and a second probe targets the second region and a third region of the target sequence. In some instances, a plurality of probes targets a single target sequence, wherein the target binding sequences of the plurality of probes contain one or more sequences which overlap with regard to complementarity to a region of the target sequence. In some instances, probe inserts do not overlap with regard to complementarity to a region of the target sequence. In some instances, at least at least 2, 10, 15, 20, 25, 30, 35, 40, 45, 50, 100, 150, 200, 300, 400, 500, 1000, 2000, 5,000, 12,000, 20,000, or more than 20,000 probes target a single target sequence. In some instances no more than 4 probes directed to a single target sequence overlap, or no more than 3, 2, 1, or no probes targeting a single target sequence overlap. In some instances, one or more probes do not target all bases in a target sequence, leaving one or more gaps. In some instances, the gaps are near the middle of the target sequence. In some instances, the gaps are at the 5′ or 3′ ends of the target sequence. In some instances, the gaps are 6 nucleotides in length. In some instances, the gaps are no more than 1, 2, 3, 4, 5, 6, 7, 8, 9, 10, 20, 30, 40, or no more than 50 nucleotides in length. In some instances, the gaps are at least 1, 2, 3, 4, 5, 6, 7, 8, 9, 10, 20, 30, 40, or at least 50 nucleotides in length. In some instances, the gap length falls within 1-50, 1-40, 1-30, 1-20, 1-10, 2-30, 2-20, 2-10, 3-50, 3-25, 3-10, or 3-8 nucleotides in length. In some instances, a set of probes targeting a sequence do not comprise overlapping regions amongst probes in the set when hybridized to complementary sequence. In some instances, a set of probes targeting a sequence do not have any gaps amongst probes in the set when hybridized to complementary sequence. Probes may be designed to maximize uniform binding to target sequences. In some instances, probes are designed to minimize target binding sequences of high or low GC content, secondary structure, repetitive/palindromic sequences, or other sequence feature that may interfere with probe binding to a target. In some instances, a single probe may target a plurality of target sequences.

A probe library described herein may comprise at least 10, 20, 50, 100, 200, 500, 1,000, 2,000, 5,000, 10,000, 20,000, 50,000, 100,000, 200,000, 500,000, 1,000,000 or more than 1,000,000 probes. A probe library may have no more than 10, 20, 50, 100, 200, 500, 1,000, 2,000, 5,000, 10,000, 20,000, 50,000, 100,000, 200,000, 500,000, or no more than 1,000,000 probes. A probe library may comprise 10 to 500, 20 to 1000, 50 to 2000, 100 to 5000, 500 to 10,000, 1,000 to 5,000, 10,000 to 50,000, 100,000 to 500,000, or 50,000 to 1,000,000 probes. A probe library may comprise about 370,000; 400,000; 500,000 or more different probes. A probe library described herein may comprise at least 2000, 5000, 10,000, 50,000, 100,000, 200,000, 500,000, 1,000,000, 2,000,000, 5,000,000, 10,000,000, 20,000,000, 50,000,000, 75,000,000, 100,000,000 or more than 200,000,000 probes. A probe library described herein may comprise about 2000, 5000, 10,000, 50,000, 100,000, 200,000, 500,000, 1,000,000, 2,000,000, 5,000,000, 10,000,000, 20,000,000, 50,000,000, 75,000,000, 100,000,000 or at least 200,000,000 probes. A probe library described herein may comprise no more than 2000, 5000, 10,000, 50,000, 100,000, 200,000, 500,000, 1,000,000, 2,000,000, 5,000,000, 10,000,000, 20,000,000, 50,000,000, 75,000,000, 100,000,000 or no more than 200,000,000 probes. A probe library may comprise 10,000 to 500,000 20,000 to 100,000, 50,000 to 200,000, 100,000 to 5,000,000, 500,000 to 10,000,000, 1,000,000 to 5,000,000, 10,000,000 to 50,000,000, 100,000 to 5,000,000, or 500,000 to 10,000,000 probes. Probe libraries in some instances comprise at least 1000, 5000, 10,000, 100,000 500,000, 1 million, 10 million, 100 million, 200 million, or at least 500 million bases. Probe libraries in some instances comprise about 1000, 5000, 10,000, 100,000, 500,000, 1 million, 10 million, 100 million, 200 million, or about 500 million bases. Probe libraries in some instances comprise 1000 to 1 million, 5000 to 1 million, 10,000 to 5 million, 100,000 to 5 million, 500,000 to 100 million, 1 million to 200 million, 10 million to 500 million, 100 million to 250 million, or 200 million to 500 million bases.

### Next Generation Sequencing Applications

Downstream applications of polynucleotide libraries may include next generation sequencing. For example, enrichment of target sequences with a controlled stoichiometry polynucleotide probe library results in more efficient sequencing. The performance of a polynucleotide library for capturing or hybridizing to targets may be defined by a number of different metrics describing efficiency, accuracy, and precision. For example, Picard metrics comprise variables such as HS library size (the number of unique molecules in the library that correspond to target regions, calculated from read pairs), mean target coverage (the percentage of bases reaching a specific coverage level), depth of coverage (number of reads including a given nucleotide) fold enrichment (sequence reads mapping uniquely to the target/reads mapping to the total sample, multiplied by the total sample length/target length), percent off-bait bases (percent of bases not corresponding to bases of the probes/baits), percent off-target (percent of bases not corresponding to bases of interest), usable bases on target, AT or GC dropout rate, fold 80 base penalty (fold over-coverage needed to raise 80 percent of non-zero targets to the mean coverage level), percent zero coverage targets, PF reads (the number of reads passing a quality filter), percent selected bases (the sum of on-bait bases and near-bait bases divided by the total aligned bases), percent duplication, or other variable consistent with the specification.

Read depth (sequencing depth, or sampling) represents the total number of times a sequenced nucleic acid fragment (a “read”) is obtained for a sequence. Theoretical read depth is defined as the expected number of times the same nucleotide is read, assuming reads are perfectly distributed throughout an idealized genome. Read depth is expressed as function of % coverage (or coverage breadth). For example, 10 million reads of a 1 million base genome, perfectly distributed, theoretically results in 10× read depth of 100% of the sequences. In practice, a greater number of reads (higher theoretical read depth, or oversampling) may be needed to obtain the desired read depth for a percentage of the target sequences. Enrichment of target sequences with a controlled stoichiometry probe library increases the efficiency of downstream sequencing, as fewer total reads will be required to obtain an outcome with an acceptable number of reads over a desired % of target sequences. For example, in some instances 55× theoretical read depth of target sequences results in at least 30× coverage of at least 90% of the sequences. In some instances no more than 55× theoretical read depth of target sequences results in at least 30× read depth of at least 80% of the sequences. In some instances no more than 55× theoretical read depth of target sequences results in at least 30× read depth of at least 95% of the sequences. In some instances no more than 55× theoretical read depth of target sequences results in at least 10× read depth of at least 98% of the sequences. In some instances, 55× theoretical read depth of target sequences results in at least 20× read depth of at least 98% of the sequences. In some instances no more than 55× theoretical read depth of target sequences results in at least 5× read depth of at least 98% of the sequences. Increasing the concentration of probes during hybridization with targets can lead to an increase in read depth. In some instances, the concentration of probes is increased by at least 1.5×, 2.0×, 2.5×, 3×, 3.5×, 4×, 5×, or more than 5×. In some instances, increasing the probe concentration results in at least a 1000% increase, or a 20%, 30%, 40%, 50%, 60%, 70%, 80%, 90%, 100%, 200%, 300%, 500%, 750%, 1000%, or more than a 1000% increase in read depth. In some instances, increasing the probe concentration by 3× results in a 1000% increase in read depth. In some instances, sequencing is performed to achieve a theoretical read depth of at least 30×, 50×, 100×, 150×, 200×, 250×, 300×, 500×, or at least 1000×. In some instances, sequencing is performed to achieve a theoretical read depth of about 30×, 50×, 100×, 150×, 200×, 250×, 300×, 500×, or about 1000×. In some instances, sequencing is performed to achieve a theoretical read depth of no more than 30×, 50×, 100×, 150×, 200×, 250×, 300×, 500×, or no more than 1000×. In some instances, sequencing is performed to achieve an actual read depth of at least 30×, 50×, 100×, 150×, 200×, 250×, 300×, 500×, or at least 1000×. In some instances, sequencing is performed to achieve an actual read depth of no more than 30×, 50×, 100×, 150×, 200×, 250×, 300×, 500×, or no more than 1000×. In some instances, sequencing is performed to achieve an actual read depth of about 30×, 50×, 100×, 150×, 200×, 250×, 300×, 500×, or about 1000×.

On-target rate represents the percentage of sequencing reads that correspond with the desired target sequences. In some instances, a controlled stoichiometry polynucleotide probe library results in an on-target rate of at least 30%, or at least 35%, 40%, 45%, 50%, 55%, 60%, 65%, 70%, 75%, 80%, 85%, or at least 90%. Increasing the concentration of polynucleotide probes during contact with target nucleic acids leads to an increase in the on-target rate. In some instances, the concentration of probes is increased by at least 1.5×, 2.0×, 2.5×, 3×, 3.5×, 4×, 5×, or more than 5×. In some instances, increasing the probe concentration results in at least a 20% increase, or a 10%, 20%, 30%, 40%, 50%, 60%, 70%, 80%, 90%, 100%, 200%, 300%, or at least a 500% increase in on-target binding. In some instances, increasing the probe concentration by 3× results in a 20% increase in on-target rate.

Coverage uniformity is in some cases calculated as the read depth as a function of the target sequence identity. Higher coverage uniformity results in a lower number of sequencing reads needed to obtain the desired read depth. For example, a property of the target sequence may affect the read depth, for example, high or low GC or AT content, repeating sequences, trailing adenines, secondary structure, affinity for target sequence binding (for amplification, enrichment, or detection), stability, melting temperature, biological activity, ability to assemble into larger fragments, sequences containing modified nucleotides or nucleotide analogues, or any other property of polynucleotides. Enrichment of target sequences with controlled stoichiometry polynucleotide probe libraries results in higher coverage uniformity after sequencing. In some instances, 95% of the sequences have a read depth that is within 1× of the mean library read depth, or about 0.05, 0.1, 0.2, 0.5, 0.7, 1, 1.2, 1.5, 1.7 or about within 2× the mean library read depth. In some instances, 80%, 85%, 90%, 95%, 97%, or 99% of the sequences have a read depth that is within 1× of the mean.

Enrichment of Target Nucleic Acids with a Polynucleotide Probe Library

A probe library described herein may be used to enrich target polynucleotides present in a population of sample polynucleotides, for a variety of downstream applications. In one some instances, a sample is obtained from one or more sources, and the population of sample polynucleotides is isolated. Samples are obtained (by way of non-limiting example) from biological sources such as saliva, blood, tissue, skin, or completely synthetic sources. The plurality of polynucleotides obtained from the sample are fragmented, end-repaired, and adenylated to form a double stranded sample nucleic acid fragment. In some instances, end repair is accomplished by treatment with one or more enzymes, such as T4 DNA polymerase, klenow enzyme, and T4 polynucleotide kinase in an appropriate buffer. A nucleotide overhang to facilitate ligation to adapters is added, in some instances with 3′ to 5′ exo minus klenow fragment and dATP.

Adapters (such as universal adapters) may be ligated to both ends of the sample polynucleotide fragments with a ligase, such as T4 ligase, to produce a library of adapter-tagged polynucleotide strands, and the adapter-tagged polynucleotide library is amplified with primers, such as universal primers. In some instances, the adapters are Y-shaped adapters comprising one or more primer binding sites, one or more grafting regions, and one or more index (or barcode) regions. In some instances, the one or more index region is present on each strand of the adapter. In some instances, grafting regions are complementary to a flowcell surface, and facilitate next generation sequencing of sample libraries. In some instances, Y-shaped adapters comprise partially complementary sequences. In some instances, Y-shaped adapters comprise a single thymidine overhang which hybridizes to the overhanging adenine of the double stranded adapter-tagged polynucleotide strands. Y-shaped adapters may comprise modified nucleic acids, that are resistant to cleavage. For example, a phosphorothioate backbone is used to attach an overhanging thymidine to the 3′ end of the adapters. If universal primers are used, amplification of the library is performed to add barcoded primers to the adapters. In some instances, an enrichment workflow is depicted in FIG. 13. A library 208 of double stranded adapter-tagged polynucleotide strands 209 is contacted with polynucleotide probes 217, to form hybrid pairs 218. Such pairs are separated 212 from unhybridized fragments, and isolated from probes to produce an enriched library 213. The enriched library may then be sequenced 214.

The library of double stranded sample nucleic acid fragments is then denatured in the presence of adapter blockers. Adapter blockers minimize off-target hybridization of probes to the adapter sequences (instead of target sequences) present on the adapter-tagged polynucleotide strands, and/or prevent intermolecular hybridization of adapters (i.e., “daisy chaining”). Denaturation is carried out in some instances at 96° C., or at about 85, 87, 90, 92, 95, 97, 98 or about 99° C. A polynucleotide targeting library (probe library) is denatured in a hybridization solution, in some instances at 96° C., at about 85, 87, 90, 92, 95, 97, 98 or 99° C. The denatured adapter-tagged polynucleotide library and the hybridization solution are incubated for a suitable amount of time and at a suitable temperature to allow the probes to hybridize with their complementary target sequences. In some instances, a suitable hybridization temperature is about 45 to 80° C., or at least 45, 50, 55, 60, 65, 70, 75, 80, 85, or 90° C. In some instances, the hybridization temperature is 70° C. In some instances, a suitable hybridization time is 16 hours, or at least 4, 6, 8, 10, 12, 14, 16, 18, 20, 22, or more than 22 hours, or about 12 to 20 hours. Binding buffer is then added to the hybridized adapter-tagged-polynucleotide probes, and a solid support comprising a capture moiety is used to selectively bind the hybridized adapter-tagged polynucleotide-probes. The solid support is washed with buffer to remove unbound polynucleotides before an elution buffer is added to release the enriched, tagged polynucleotide fragments from the solid support. In some instances, the solid support is washed 2 times, or 1, 2, 3, 4, 5, or 6 times. The enriched library of adapter-tagged polynucleotide fragments is amplified and the enriched library is sequenced.

A plurality of nucleic acids (i.e. genomic sequence) may obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. The adapter-tagged polynucleotide library is then denatured at high temperature, preferably 96° C., in the presence of adapter blockers. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched library of adapter-tagged polynucleotide fragments is amplified and then the library is sequenced. Alternative variables such as incubation times, temperatures, reaction volumes/concentrations, number of washes, or other variables consistent with the specification are also employed in the method.

In any of the instances, the detection or quantification analysis of the oligonucleotides can be accomplished by sequencing. The subunits or entire synthesized oligonucleotides can be detected via full sequencing of all oligonucleotides by any suitable methods known in the art, e.g., Illumina sequencing by synthesis, PacBio nanopore sequencing, or BGI/MGI nanoball sequencing, including the sequencing methods described herein.

Sequencing can be accomplished through classic Sanger sequencing methods which are well known in the art. Sequencing can also be accomplished using high-throughput systems some of which allow detection of a sequenced nucleotide immediately after or upon its incorporation into a growing strand, i.e., detection of sequence in red time or substantially real time. In some cases, high throughput sequencing generates at least 1,000, at least 5,000, at least 10,000, at least 20,000, at least 30,000, at least 40,000, at least 50,000, at least 100,000 or at least 500,000 sequence reads per hour; with each read being at least 50, at least 60, at least 70, at least 80, at least 90, at least 100, at least 120 or at least 150 bases per read.

In some instances, high-throughput sequencing involves the use of technology available by Illumina's Genome Analyzer IIX, MiSeq personal sequencer, or HiSeq systems, such as those using HiSeq 2500, HiSeq 1500, HiSeq 2000, HiSeq 1000, iSeq 100, Mini Seq, MiSeq, NextSeq 550, NextSeq 2000, NextSeq 550, or NovaSeq 6000. These machines use reversible terminator-based sequencing by synthesis chemistry. These machines can generate 6000 Gb or more reads in 13-44 hours. Smaller systems may be utilized for runs within 3, 2, 1 days or less time. Short synthesis cycles may be used to minimize the time it takes to obtain sequencing results.

In some instances, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM) can do 10 million reads in two hours.

In some instances, high-throughput sequencing involves the use of technology available by Helicos BioSciences Corporation (Cambridge, Mass.) such as the Single Molecule Sequencing by Synthesis (SMSS) method. SMSS is unique because it allows for sequencing the entire human genome in up to 24 hours. Finally, SMSS is powerful because, like the MW technology, it does not require a pre amplification step prior to hybridization. In fact, SMSS does not require any amplification.

In some instances, high-throughput sequencing involves the use of technology available by 454 Lifesciences, Inc. (Branford, Conn.) such as the Pico Titer Plate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles, M., et al. “Genome sequencing in microfabricated high-density picolitre reactors”, Nature, doi: 10.1038/nature03959.

In some instances, high-throughput sequencing is performed using Clonal Single Molecule Array (Solexa, Inc.) or sequencing-by-synthesis (SBS) utilizing reversible terminator chemistry. Constans, A., The Scientist 2003, 17(13):36. High-throughput sequencing of oligonucleotides can be achieved using any suitable sequencing method known in the art, such as those commercialized by Pacific Biosciences, Complete Genomics, Genia Technologies, Halcyon Molecular, Oxford Nanopore Technologies and the like. Overall such systems involve sequencing a target oligonucleotide molecule having a plurality of bases by the temporal addition of bases via a polymerization reaction that is measured on a molecule of oligonucleotide, i e., the activity of a nucleic acid polymerizing enzyme on the template oligonucleotide molecule to be sequenced is followed in real time. Sequence can then be deduced by identifying which base is being incorporated into the growing complementary strand of the target oligonucleotide by the catalytic activity of the nucleic acid polymerizing enzyme at each step in the sequence of base additions. A polymerase on the target oligonucleotide molecule complex is provided in a position suitable to move along the target oligonucleotide molecule and extend the oligonucleotide primer at an active site. A plurality of labeled types of nucleotide analogs are provided proximate to the active site, with each distinguishably type of nucleotide analog being complementary to a different nucleotide in the target oligonucleotide sequence. The growing oligonucleotide strand is extended by using the polymerase to add a nucleotide analog to the oligonucleotide strand at the active site, where the nucleotide analog being added is complementary to the nucleotide of the target oligonucleotide at the active site. The nucleotide analog added to the oligonucleotide primer as a result of the polymerizing step is identified. The steps of providing labeled nucleotide analogs, polymerizing the growing oligonucleotide strand, and identifying the added nucleotide analog are repeated so that the oligonucleotide strand is further extended and the sequence of the target oligonucleotide is determined.

The next generation sequencing technique can comprises real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (10″ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing {See e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridION system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNₓ, or SiO₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamethyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

A population of polynucleotides may be enriched prior to adapter ligation. In one example, a plurality of polynucleotides is obtained from a sample, fragmented, optionally end-repaired, and denatured at high temperature, preferably 90-99° C. A polynucleotide targeting library (probe library) is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 2 and 5 times to remove unbound polynucleotides before an elution buffer is added to release the enriched, adapter-tagged polynucleotide fragments from the solid support. The enriched polynucleotide fragments are then polyadenylated, adapters are ligated to both ends of the polynucleotide fragments to produce a library of adapter-tagged polynucleotide strands, and the adapter-tagged polynucleotide library is amplified. The adapter-tagged polynucleotide library is then sequenced.

A polynucleotide targeting library may also be used to filter undesired sequences from a plurality of polynucleotides, by hybridizing to undesired fragments. For example, a plurality of polynucleotides is obtained from a sample, and fragmented, optionally end-repaired, and adenylated. Adapters are ligated to both ends of the polynucleotide fragments to produce a library of adapter-tagged polynucleotide strands, and the adapter-tagged polynucleotide library is amplified. Alternatively, adenylation and adapter ligation steps are instead performed after enrichment of the sample polynucleotides. The adapter-tagged polynucleotide library is then denatured at high temperature, preferably 90-99° C., in the presence of adapter blockers. A polynucleotide filtering library (probe library) designed to remove undesired, non-target sequences is denatured in a hybridization solution at high temperature, preferably about 90 to 99° C., and combined with the denatured, tagged polynucleotide library in hybridization solution for about 10 to 24 hours at about 45 to 80° C. Binding buffer is then added to the hybridized tagged polynucleotide probes, and a solid support comprising a capture moiety are used to selectively bind the hybridized adapter-tagged polynucleotide-probes. The solid support is washed one or more times with buffer, preferably about 1 and 5 times to elute unbound adapter-tagged polynucleotide fragments. The enriched library of unbound adapter-tagged polynucleotide fragments is amplified and then the amplified library is sequenced.

### Highly Parallel De Novo Nucleic Acid Synthesis

Described herein is a platform approach utilizing miniaturization, parallelization, and vertical integration of the end-to-end process from polynucleotide synthesis to gene assembly within Nano wells on silicon to create a revolutionary synthesis platform. Devices described herein provide, with the same footprint as a 96-well plate, a silicon synthesis platform is capable of increasing throughput by a factor of 100 to 1,000 compared to traditional synthesis methods, with production of up to approximately 1,000,000 polynucleotides in a single highly-parallelized run. In some instances, a single silicon plate described herein provides for synthesis of about 6,100 non-identical polynucleotides. In some instances, each of the non-identical polynucleotides is located within a cluster. A cluster may comprise 50 to 500 non-identical polynucleotides.

Methods described herein provide for synthesis of a library of polynucleotides each encoding for a predetermined variant of at least one predetermined reference nucleic acid sequence. In some cases, the predetermined reference sequence is nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. The synthesized specific alterations in the nucleic acid sequence can be introduced by incorporating nucleotide changes into overlapping or blunt ended polynucleotide primers. Alternatively, a population of polynucleotides may collectively encode for a long nucleic acid (e.g., a gene) and variants thereof. In this arrangement, the population of polynucleotides can be hybridized and subject to standard molecular biology techniques to form the long nucleic acid (e.g., a gene) and variants thereof. When the long nucleic acid (e.g., a gene) and variants thereof are expressed in cells, a variant protein library is generated. Similarly, provided here are methods for synthesis of variant libraries encoding for RNA sequences (e.g., miRNA, shRNA, and mRNA) or DNA sequences (e.g., enhancer, promoter, UTR, and terminator regions). Also provided here are downstream applications for variants selected out of the libraries synthesized using methods described here. Downstream applications include identification of variant nucleic acid or protein sequences with enhanced biologically relevant functions, e.g., biochemical affinity, enzymatic activity, changes in cellular activity, and for the treatment or prevention of a disease state.

### Substrates

Provided herein are substrates comprising a plurality of clusters, wherein each cluster comprises a plurality of loci that support the attachment and synthesis of polynucleotides. The term “locus” as used herein refers to a discrete region on a structure which provides support for polynucleotides encoding for a single predetermined sequence to extend from the surface. In some instances, a locus is on a two dimensional surface, e.g., a substantially planar surface. In some instances, a locus refers to a discrete raised or lowered site on a surface e.g., a well, micro well, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some instances, a surface of a device is inclusive of one or a plurality of surfaces of a substrate.

Provided herein are structures that may comprise a surface that supports the synthesis of a plurality of polynucleotides having different predetermined sequences at addressable locations on a common support. In some instances, a device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some instances, the device provides support for the synthesis of more than 2,000; 5,000; 10,000; 20,000; 30,000; 50,000; 75,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence.

Provided herein are methods and devices for manufacture and growth of polynucleotides about 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, or 2000 bases in length. In some instances, the length of the polynucleotide formed is about 5, 10, 20, 30, 40, 50, 60, 70, 80, 90, 100, 125, 150, 175, 200, or 225 bases in length. A polynucleotide may be at least 5, 10, 20, 30, 40, 50, 60, 70, 80, 90, or 100 bases in length. A polynucleotide may be from 10 to 225 bases in length, from 12 to 100 bases in length, from 20 to 150 bases in length, from 20 to 130 bases in length, or from 30 to 100 bases in length.

In some instances, polynucleotides are synthesized on distinct loci of a substrate, wherein each locus supports the synthesis of a population of polynucleotides. In some instances, each locus supports the synthesis of a population of polynucleotides having a different sequence than a population of polynucleotides grown on another locus. In some instances, the loci of a device are located within a plurality of clusters. In some instances, a device comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a device comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a device comprises about 10,000 distinct loci. The amount of loci within a single cluster is varied in different instances. In some instances, each cluster includes 1, 2, 3, 4, 5, 6, 7, 8, 9, 10, 20, 30, 40, 50, 60, 70, 80, 90, 100, 120, 130, 150, 200, 300, 400, 500, 1000 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci.

The number of distinct polynucleotides synthesized on a device may be dependent on the number of distinct loci available in the substrate. In some instances, the density of loci within a cluster of a device is at least or about 1 locus per mm², 10 loci per mm², 25 loci per mm², 50 loci per mm², 65 loci per mm², 75 loci per mm², 100 loci per mm², 130 loci per mm², 150 loci per mm², 175 loci per mm², 200 loci per mm², 300 loci per mm², 400 loci per mm², 500 loci per mm², 1,000 loci per mm² or more. In some instances, a device comprises from about 10 loci per mm² to about 500 mm², from about 25 loci per mm² to about 400 mm², from about 50 loci per mm² to about 500 mm², from about 100 loci per mm² to about 500 mm², from about 150 loci per mm² to about 500 mm², from about 10 loci per mm² to about 250 mm², from about 50 loci per mm² to about 250 mm², from about 10 loci per mm² to about 200 mm², or from about 50 loci per mm² to about 200 mm². In some instances, the distance from the centers of two adjacent loci within a cluster is from about 10 μm to about 500 μm, from about 10 μm to about 200 μm, or from about 10 μm to about 100 μm. In some instances, the distance from two centers of adjacent loci is greater than about 10 μm, 20 um, 30 μm, 40 um, 50 μm, 60 um, 70 μm, 80 um, 90 μm or 100 μm. In some instances, the distance from the centers of two adjacent loci is less than about 200 μm, 150 um, 100 μm, 80 um, 70 μm, 60 um, 50 μm, 40 um, 30 μm, 20 μm or 10 μm. In some instances, each locus has a width of about 0.5 um, 1 μm, 2 um, 3 μm, 4 um, 5 μm, 6 um, 7 μm, 8 um, 9 μm, 10 um, 20 μm, 30 um, 40 μm, 50 um, 60 μm, 70 um, 80 μm, 90 μm or 100 μm. In some instances, each locus is has a width of about 0.5 um to 100 μm, about 0.5 um to 50 μm, about 10 μm to 75 μm, or about 0.5 um to 50 um.

In some instances, the density of clusters within a device is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a device comprises from about 1 cluster per 10 mm² to about 10 clusters per 1 mm². In some instances, the distance from the centers of two adjacent clusters is less than about 50 μm, 100 um, 200 μm, 500 um, 1000 μm, or 2000 μm or 5000 μm. In some instances, the distance from the centers of two adjacent clusters is from about 50 μm and about 100 μm, from about 50 μm and about 200 μm, from about 50 μm and about 300 μm, from about 50 μm and about 500 μm, and from about 100 μm to about 2000 μm. In some instances, the distance from the centers of two adjacent clusters is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, each cluster has a diameter or width along one dimension of about 0.5 to 2 mm, about 0.5 to 1 mm, or about 1 to 2 mm. In some instances, each cluster has a diameter or width along one dimension of about 0.5, 0.6, 0.7, 0.8, 0.9, 1, 1.1, 1.2, 1.3, 1.4, 1.5, 1.6, 1.7, 1.8, 1.9 or 2 mm. In some instances, each cluster has an interior diameter or width along one dimension of about 0.5, 0.6, 0.7, 0.8, 0.9, 1, 1.1, 1.15, 1.2, 1.3, 1.4, 1.5, 1.6, 1.7, 1.8, 1.9 or 2 mm.

A device may be about the size of a standard 96 well plate, for example from about 100 and 200 mm by from about 50 and 150 mm. In some instances, a device has a diameter less than or equal to about 1000 mm, 500 mm, 450 mm, 400 mm, 300 mm, 250 nm, 200 mm, 150 mm, 100 mm or 50 mm. In some instances, the diameter of a device is from about 25 mm and 1000 mm, from about 25 mm and about 800 mm, from about 25 mm and about 600 mm, from about 25 mm and about 500 mm, from about 25 mm and about 400 mm, from about 25 mm and about 300 mm, or from about 25 mm and about 200. Non-limiting examples of device size include about 300 mm, 200 mm, 150 mm, 130 mm, 100 mm, 76 mm, 51 mm and 25 mm. In some instances, a device has a planar surface area of at least about 100 mm²; 200 mm²; 500 mm²; 1,000 mm²; 2,000 mm²; 5,000 mm²; 10,000 mm²; 12,000 mm²; 15,000 mm²; 20,000 mm²; 30,000 mm²; 40,000 mm²; 50,000 mm² or more. In some instances, the thickness of a device is from about 50 mm and about 2000 mm, from about 50 mm and about 1000 mm, from about 100 mm and about 1000 mm, from about 200 mm and about 1000 mm, or from about 250 mm and about 1000 mm. Non-limiting examples of device thickness include 275 mm, 375 mm, 525 mm, 625 mm, 675 mm, 725 mm, 775 mm and 925 mm. In some instances, the thickness of a device varies with diameter and depends on the composition of the substrate. For example, a device comprising materials other than silicon has a different thickness than a silicon device of the same diameter. Device thickness may be determined by the mechanical strength of the material used and the device must be thick enough to support its own weight without cracking during handling. In some instances, a structure comprises a plurality of devices described herein.

### Surface Materials

Provided herein is a device comprising a surface, wherein the surface is modified to support polynucleotide synthesis at predetermined locations and with a resulting low error rate, a low dropout rate, a high yield, and a high oligo representation. In some instances, surfaces of a device for polynucleotide synthesis provided herein are fabricated from a variety of materials capable of modification to support a de novo polynucleotide synthesis reaction. In some cases, the devices are sufficiently conductive, e.g., are able to form uniform electric fields across all or a portion of the device. A device described herein may comprise a flexible material. Exemplary flexible materials include, without limitation, modified nylon, unmodified nylon, nitrocellulose, and polypropylene. A device described herein may comprise a rigid material. Exemplary rigid materials include, without limitation, glass, fuse silica, silicon, silicon dioxide, silicon nitride, plastics (for example, polytetrafluoroethylene, polypropylene, polystyrene, polycarbonate, and blends thereof, and metals (for example, gold, platinum). Device disclosed herein may be fabricated from a material comprising silicon, polystyrene, agarose, dextran, cellulosic polymers, polyacrylamides, polydimethylsiloxane (PDMS), glass, or any combination thereof. In some cases, a device disclosed herein is manufactured with a combination of materials listed herein or any other suitable material known in the art.

A listing of tensile strengths for exemplary materials described herein is provides as follows: nylon (70 MPa), nitrocellulose (1.5 MPa), polypropylene (40 MPa), silicon (268 MPa), polystyrene (40 MPa), agarose (1-10 MPa), polyacrylamide (1-10 MPa), polydimethylsiloxane (PDMS) (3.9-10.8 MPa). Solid supports described herein can have a tensile strength from 1 to 300, 1 to 40, 1 to 10, 1 to 5, or 3 to 11 MPa. Solid supports described herein can have a tensile strength of about 1, 1.5, 2, 3, 4, 5, 6, 7, 8, 9, 10, 11, 20, 25, 40, 50, 60, 70, 80, 90, 100, 150, 200, 250, 270, or more MPa. In some instances, a device described herein comprises a solid support for polynucleotide synthesis that is in the form of a flexible material capable of being stored in a continuous loop or reel, such as a tape or flexible sheet.

Young's modulus measures the resistance of a material to elastic (recoverable) deformation under load. A listing of Young's modulus for stiffness of exemplary materials described herein is provides as follows: nylon (3 GPa), nitrocellulose (1.5 GPa), polypropylene (2 GPa), silicon (150 GPa), polystyrene (3 GPa), agarose (1-10 GPa), polyacrylamide (1-10 GPa), polydimethylsiloxane (PDMS) (1-10 GPa). Solid supports described herein can have a Young's moduli from 1 to 500, 1 to 40, 1 to 10, 1 to 5, or 3 to 11 GPa. Solid supports described herein can have a Young's moduli of about 1, 1.5, 2, 3, 4, 5, 6, 7, 8, 9, 10, 11, 20, 25, 40, 50, 60, 70, 80, 90, 100, 150, 200, 250, 400, 500 GPa, or more. As the relationship between flexibility and stiffness are inverse to each other, a flexible material has a low Young's modulus and changes its shape considerably under load.

In some cases, a device disclosed herein comprises a silicon dioxide base and a surface layer of silicon oxide. Alternatively, the device may have a base of silicon oxide. Surface of the device provided here may be textured, resulting in an increase overall surface area for polynucleotide synthesis. Device disclosed herein may comprise at least 5%, 10%, 25%, 50%, 80%, 90%, 95%, or 99% silicon. A device disclosed herein may be fabricated from a silicon on insulator (SOI) wafer.

### Surface Architecture

Provided herein are devices comprising raised and/or lowered features. One benefit of having such features is an increase in surface area to support polynucleotide synthesis. In some instances, a device having raised and/or lowered features is referred to as a three-dimensional substrate. In some instances, a three-dimensional device comprises one or more channels. In some instances, one or more loci comprise a channel. In some instances, the channels are accessible to reagent deposition via a deposition device such as a polynucleotide synthesizer. In some instances, reagents and/or fluids collect in a larger well in fluid communication one or more channels. For example, a device comprises a plurality of channels corresponding to a plurality of loci with a cluster, and the plurality of channels are in fluid communication with one well of the cluster. In some methods, a library of polynucleotides is synthesized in a plurality of loci of a cluster.

In some instances, the structure is configured to allow for controlled flow and mass transfer paths for polynucleotide synthesis on a surface. In some instances, the configuration of a device allows for the controlled and even distribution of mass transfer paths, chemical exposure times, and/or wash efficacy during polynucleotide synthesis. In some instances, the configuration of a device allows for increased sweep efficiency, for example by providing sufficient volume for a growing a polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are methods to synthesize an amount of DNA of 1 fM, 5 fM, 10 fM, 25 fM, 50 fM, 75 fM, 100 fM, 200 fM, 300 fM, 400 fM, 500 fM, 600 fM, 700 fM, 800 fM, 900 fM, 1 pM, 5 pM, 10 pM, 25 pM, 50 pM, 75 pM, 100 pM, 200 pM, 300 pM, 400 pM, 500 pM, 600 pM, 700 pM, 800 pM, 900 pM, or more. In some instances, a polynucleotide library may span the length of about 1%, 2%, 3%, 4%, 5%, 10%, 15%, 20%, 30%, 40%, 50%, 60%, 70%, 80%, 90%, 95%, or 100% of a gene. A gene may be varied up to about 1%, 2%, 3%, 4%, 5%, 10%, 15%, 20%, 30%, 40%, 50%, 60%, 70%, 80%, 85%, 90%, 95%, or 100%.

Non-identical polynucleotides may collectively encode a sequence for at least 1%, 2%, 3%, 4%, 5%, 10%, 15%, 20%, 30%, 40%, 50%, 60%, 70%, 80%, 85%, 90%, 95%, or 100% of a gene. In some instances, a polynucleotide may encode a sequence of 50%, 60%, 70%, 80%, 85%, 90%, 95%, or more of a gene. In some instances, a polynucleotide may encode a sequence of 80%, 85%, 90%, 95%, or more of a gene.

In some instances, segregation is achieved by physical structure. In some instances, segregation is achieved by differential functionalization of the surface generating active and passive regions for polynucleotide synthesis. Differential functionalization is also be achieved by alternating the hydrophobicity across the device surface, thereby creating water contact angle effects that cause beading or wetting of the deposited reagents. Employing larger structures can decrease splashing and cross-contamination of distinct polynucleotide synthesis locations with reagents of the neighboring spots. In some instances, a device, such as a polynucleotide synthesizer, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000; 1:3,000; 1:5,000; or 1:10,000). In some instances, a device comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a device may have the same or different width, height, and/or volume as another well of the substrate. A channel of a device may have the same or different width, height, and/or volume as another channel of the substrate. In some instances, the width of a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a well comprising a cluster is from about 0.05 mm to about 50 mm, from about 0.05 mm to about 10 mm, from about 0.05 mm and about 5 mm, from about 0.05 mm and about 4 mm, from about 0.05 mm and about 3 mm, from about 0.05 mm and about 2 mm, from about 0.05 mm and about 1 mm, from about 0.05 mm and about 0.5 mm, from about 0.05 mm and about 0.1 mm, from about 0.1 mm and 10 mm, from about 0.2 mm and 10 mm, from about 0.3 mm and about 10 mm, from about 0.4 mm and about 10 mm, from about 0.5 mm and 10 mm, from about 0.5 mm and about 5 mm, or from about 0.5 mm and about 2 mm. In some instances, the width of a cluster is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a cluster is from about 1.0 and 1.3 mm. In some instances, the width of a cluster is about 1.150 mm. In some instances, the width of a well is less than or about 5 mm, 4 mm, 3 mm, 2 mm, 1 mm, 0.5 mm, 0.1 mm, 0.09 mm, 0.08 mm, 0.07 mm, 0.06 mm or 0.05 mm. In some instances, the width of a well is from about 1.0 and 1.3 mm. In some instances, the width of a well is about 1.150 mm. In some instances, the width of a cluster is about 0.08 mm. In some instances, the width of a well is about 0.08 mm. The width of a cluster may refer to clusters within a two-dimensional or three-dimensional substrate.

In some instances, the height of a well is from about 20 um to about 1000 um, from about 50 um to about 1000 um, from about 100 um to about 1000 um, from about 200 um to about 1000 um, from about 300 um to about 1000 um, from about 400 um to about 1000 um, or from about 500 um to about 1000 um. In some instances, the height of a well is less than about 1000 um, less than about 900 um, less than about 800 um, less than about 700 um, or less than about 600 um.

In some instances, a device comprises a plurality of channels corresponding to a plurality of loci within a cluster, wherein the height or depth of a channel is from about 5 um to about 500 um, from about 5 um to about 400 um, from about 5 um to about 300 um, from about 5 um to about 200 um, from about 5 um to about 100 um, from about 5 um to about 50 um, or from about 10 um to about 50 um. In some instances, the height of a channel is less than 100 um, less than 80 um, less than 60 um, less than 40 um or less than 20 um.

In some instances, the diameter of a channel, locus (e.g., in a substantially planar substrate) or both channel and locus (e.g., in a three-dimensional device wherein a locus corresponds to a channel) is from about 1 um to about 1000 um, from about 1 um to about 500 um, from about 1 um to about 200 um, from about 1 um to about 100 um, from about 5 um to about 100 um, or from about 10 um to about 100 um, for example, about 90 um, 80 um, 70 um, 60 um, 50 um, 40 um, 30 um, 20 um or 10 um. In some instances, the diameter of a channel, locus, or both channel and locus is less than about 100 um, 90 um, 80 um, 70 um, 60 um, 50 um, 40 um, 30 um, 20 um or 10 um. In some instances, the distance from the center of two adjacent channels, loci, or channels and loci is from about 1 um to about 500 um, from about 1 um to about 200 um, from about 1 um to about 100 um, from about 5 um to about 200 um, from about 5 um to about 100 um, from about 5 um to about 50 um, or from about 5 um to about 30 um, for example, about 20 um.

### Surface Modifications

In various instances, surface modifications are employed for the chemical and/or physical alteration of a surface by an additive or subtractive process to change one or more chemical and/or physical properties of a device surface or a selected site or region of a device surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some instances, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some instances, the adhesion promoter is a chemical with a high surface energy. In some instances, a second chemical layer is deposited on a surface of a substrate. In some instances, the second chemical layer has a low surface energy. In some instances, surface energy of a chemical layer coated on a surface supports localization of droplets on the surface. Depending on the patterning arrangement selected, the proximity of loci and/or area of fluid contact at the loci are alterable.

In some instances, a device surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a device surface is modified with one or more different layers of compounds. Such modification layers of interest include, without limitation, inorganic and organic layers such as metals, metal oxides, polymers, small organic molecules and the like. Non-limiting polymeric layers include peptides, proteins, nucleic acids or mimetics thereof (e.g., peptide nucleic acids and the like), polysaccharides, phospholipids, polyurethanes, polyesters, polycarbonates, polyureas, polyamides, polyethyleneamines, polyarylene sulfides, polysiloxanes, polyimides, polyacetates, and any other suitable compounds described herein or otherwise known in the art. In some instances, polymers are heteropolymeric. In some instances, polymers are homopolymeric. In some instances, polymers comprise functional moieties or are conjugated.

In some instances, resolved loci of a device are functionalized with one or more moieties that increase and/or decrease surface energy. In some instances, a moiety is chemically inert. In some instances, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for device functionalization may comprise: (a) providing a device having a surface that comprises silicon dioxide; and (b) silanizing the surface using, a suitable silanizing agent described herein or otherwise known in the art, for example, an organofunctional alkoxysilane molecule.

In some instances, the organofunctional alkoxysilane molecule comprises dimethylchloro-octodecyl-silane, methyldichloro-octodecyl-silane, trichloro-octodecyl-silane, trimethyl-octodecyl-silane, triethyl-octodecyl-silane, or any combination thereof. In some instances, a device surface comprises functionalized with polyethylene/polypropylene (functionalized by gamma irradiation or chromic acid oxidation, and reduction to hydroxyalkyl surface), highly crosslinked polystyrene-divinylbenzene (derivatized by chloromethylation, and aminated to benzylamine functional surface), nylon (the terminal aminohexyl groups are directly reactive), or etched with reduced polytetrafluoroethylene. Other methods and functionalizing agents are described in U.S. Pat. No. 5,474,796, which is herein incorporated by reference in its entirety.

In some instances, a device surface is functionalized by contact with a derivatizing composition that contains a mixture of silanes, under reaction conditions effective to couple the silanes to the device surface, typically via reactive hydrophilic moieties present on the device surface. Silanization generally covers a surface through self-assembly with organofunctional alkoxysilane molecules.

A variety of siloxane functionalizing reagents can further be used as currently known in the art, e.g., for lowering or increasing surface energy. The organofunctional alkoxysilanes can be classified according to their organic functions.

Provided herein are devices that may contain patterning of agents capable of coupling to a nucleoside. In some instances, a device may be coated with an active agent. In some instances, a device may be coated with a passive agent. Exemplary active agents for inclusion in coating materials described herein includes, without limitation, N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), 3-iodo-propyltrimethoxysilane, butyl-aldehydr-trimethoxysilane, dimeric secondary aminoalkyl siloxanes, (3-aminopropyl)-diethoxy-methylsilane, (3-aminopropyl)-dimethyl-ethoxysilane, and (3-aminopropyl)-trimethoxysilane, (3-glycidoxypropyl)-dimethyl-ethoxysilane, glycidoxy-trimethoxysilane, (3-mercaptopropyl)-trimethoxysilane, 3-4 epoxycyclohexyl-ethyltrimethoxysilane, and (3-mercaptopropyl)-methyl-dimethoxysilane, allyl trichlorochlorosilane, 7-oct-1-enyl trichlorochlorosilane, or bis (3-trimethoxysilylpropyl) amine.

Exemplary passive agents for inclusion in a coating material described herein includes, without limitation, perfluorooctyltrichlorosilane; tridecafluoro-1,1,2,2-tetrahydrooctyl)trichlorosilane; 1H, 1H, 2H, 2H-fluorooctyltriethoxysilane (FOS); trichloro(1H, 1H, 2H, 2H-perfluorooctyl)silane; tert-butyl-[5-fluoro-4-(4,4,5,5-tetramethyl-1,3,2-dioxaborolan-2-yl)indol-1-yl]-dimethyl-silane; CYTOP™; Fluorinert™; perfluoroctyltrichlorosilane (PFOTCS); perfluorooctyldimethylchlorosilane (PFODCS); perfluorodecyltriethoxysilane (PFDTES); pentafluorophenyl-dimethylpropylchloro-silane (PFPTES); perfluorooctyltriethoxysilane; perfluorooctyltrimethoxysilane; octylchlorosilane; dimethylchloro-octodecyl-silane; methyldichloro-octodecyl-silane; trichloro-octodecyl-silane; trimethyl-octodecyl-silane; triethyl-octodecyl-silane; or octadecyltrichlorosilane.

In some instances, a functionalization agent comprises a hydrocarbon silane such as octadecyltrichlorosilane. In some instances, the functionalizing agent comprises 11-acetoxyundecyltriethoxysilane, n-decyltriethoxysilane, (3-aminopropyl)trimethoxysilane, (3-aminopropyl)triethoxysilane, glycidyloxypropyl/trimethoxysilane and N-(3-triethoxysilylpropyl)-4-hydroxybutyramide.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 minutes, 1 minute, 50 seconds, 40 seconds, 30 seconds, 20 seconds and 10 seconds.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′—OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with 12/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I2/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises the phosphite triester is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)-(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide is optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example at least or about at least 2,3,4,5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides are synthesized on a device with low error rates described herein in less than about three months, two months, one month, three weeks, 15, 14, 13, 12, 11, 10, 9, 8, 7, 6, 5, 4, 3, 2 days, 24 hours or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours or less.

In some instances, methods described herein provide for generation of a library of polynucleotides comprising variant polynucleotides differing at a plurality of codon sites. In some instances, a polynucleotide may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent and separated by 1, 2, 3, 4, 5, 6, 7, 8, 9, 10, or more codons.

In some instances, a polynucleotide may comprise multiple sites of variant codon sites, wherein all the variant codon sites are adjacent to one another, forming a stretch of variant codon sites. In some instances, a polynucleotide may comprise multiple sites of variant codon sites, wherein none the variant codon sites are adjacent to one another. In some instances, a polynucleotide may comprise multiple sites of variant codon sites, wherein some the variant codon sites are adjacent to one another, forming a stretch of variant codon sites, and some of the variant codon sites are not adjacent to one another.

### Large Polynucleotide Libraries Having Low Error Rates

Average error rates for polynucleotides synthesized within a library using the systems and methods provided may be less than 1 in 1000, less than 1 in 1250, less than 1 in 1500, less than 1 in 2000, less than 1 in 3000 or less often. In some instances, average error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less. In some instances, average error rates for polynucleotides synthesized within a library using the systems and methods provided are less than 1/1000.

In some instances, aggregate error rates for polynucleotides synthesized within a library using the systems and methods provided are less than 1/500, 1/600, 1/700, 1/800, 1/900, 1/1000, 1/1100, 1/1200, 1/1250, 1/1300, 1/1400, 1/1500, 1/1600, 1/1700, 1/1800, 1/1900, 1/2000, 1/3000, or less compared to the predetermined sequences. In some instances, aggregate error rates for polynucleotides synthesized within a library using the systems and methods provided are less than 1/500, 1/600, 1/700, 1/800, 1/900, or 1/1000. In some instances, aggregate error rates for polynucleotides synthesized within a library using the systems and methods provided are less than 1/1000.

In some instances, an error correction enzyme may be used for polynucleotides synthesized within a library using the systems and methods provided can use. In some instances, aggregate error rates for polynucleotides with error correction can be less than 1/500, 1/600, 1/700, 1/800, 1/900, 1/1000, 1/1100, 1/1200, 1/1300, 1/1400, 1/1500, 1/1600, 1/1700, 1/1800, 1/1900, 1/2000, 1/3000, or less compared to the predetermined sequences. In some instances, aggregate error rates with error correction for polynucleotides synthesized within a library using the systems and methods provided can be less than 1/500, 1/600, 1/700, 1/800, 1/900, or 1/1000. In some instances, aggregate error rates with error correction for polynucleotides synthesized within a library using the systems and methods provided can be less than 1/1000.

Error rate may limit the value of gene synthesis for the production of libraries of gene variants. With an error rate of 1/300, about 0.7% of the clones in a 1500 base pair gene will be correct. As most of the errors from polynucleotide synthesis result in frame-shift mutations, over 99% of the clones in such a library will not produce a full-length protein. Reducing the error rate by 75% would increase the fraction of clones that are correct by a factor of 40. The methods and compositions of the disclosure allow for fast de novo synthesis of large polynucleotide and gene libraries with error rates that are lower than commonly observed gene synthesis methods both due to the improved quality of synthesis and the applicability of error correction methods that are enabled in a massively parallel and time-efficient manner. Accordingly, libraries may be synthesized with base insertion, deletion, substitution, or total error rates that are under 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less, across the library, or across more than 80%, 85%, 90%, 93%, 95%, 96%, 97%, 98%, 99%, 99.5%, 99.8%, 99.9%, 99.95%, 99.98%, 99.99%, or more of the library. The methods and compositions of the disclosure further relate to large synthetic polynucleotide and gene libraries with low error rates associated with at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in at least a subset of the library to relate to error free sequences in comparison to a predetermined/preselected sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of the polynucleotides or genes in an isolated volume within the library have the same sequence. In some instances, at least 30%, 40%, 50%, 60%, 70%, 75%, 80%, 85%, 90%, 93%, 95%, 96%, 97%, 98%, 99%, 99.5%, 99.8%, 99.9%, 99.95%, 99.98%, 99.99%, or more of any polynucleotides or genes related with more than 95%, 96%, 97%, 98%, 99%, 99.5%, 99.6%, 99.7%, 99.8%, 99.9% or more similarity or identity have the same sequence. In some instances, the error rate related to a specified locus on a polynucleotide or gene is optimized. Thus, a given locus or a plurality of selected loci of one or more polynucleotides or genes as part of a large library may each have an error rate that is less than 1/300, 1/400, 1/500, 1/600, 1/700, 1/800, 1/900, 1/1000, 1/1250, 1/1500, 1/2000, 1/2500, 1/3000, 1/4000, 1/5000, 1/6000, 1/7000, 1/8000, 1/9000, 1/10000, 1/12000, 1/15000, 1/20000, 1/25000, 1/30000, 1/40000, 1/50000, 1/60000, 1/70000, 1/80000, 1/90000, 1/100000, 1/125000, 1/150000, 1/200000, 1/300000, 1/400000, 1/500000, 1/600000, 1/700000, 1/800000, 1/900000, 1/1000000, or less. In various instances, such error optimized loci may comprise at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 50000, 75000, 100000, 500000, 1000000, 2000000, 3000000 or more loci. The error optimized loci may be distributed to at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900, 1000, 1500, 2000, 2500, 3000, 4000, 5000, 6000, 7000, 8000, 9000, 10000, 30000, 75000, 100000, 500000, 1000000, 2000000, 3000000 or more polynucleotides or genes.

The error rates can be achieved with or without error correction. The error rates can be achieved across the library, or across more than 80%, 85%, 90%, 93%, 95%, 96%, 97%, 98%, 99%, 99.5%, 99.8%, 99.9%, 99.95%, 99.98%, 99.99%, or more of the library.

### Computer Systems

Any of the systems described herein, may be operably linked to a computer and may be automated through a computer either locally or remotely. In various instances, the methods and systems of the disclosure may further comprise software programs on computer systems and use thereof. Accordingly, computerized control for the synchronization of the dispense/vacuum/refill functions such as orchestrating and synchronizing the material deposition device movement, dispense action and vacuum actuation are within the bounds of the disclosure. The computer systems may be programmed to interface between the user specified base sequence and the position of a material deposition device to deliver the correct reagents to specified regions of the substrate.

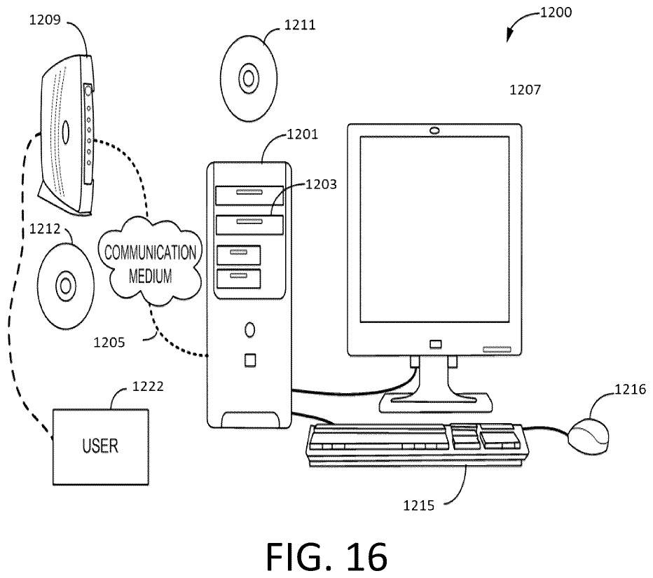

The computer system 1200 illustrated in FIG. 16 may be understood as a logical apparatus that can read instructions from media 1211 and/or a network port 1205, which can optionally be connected to server 1209 having fixed media 1212. The system, such as shown in FIG. 16 can include a CPU 1201, disk drives 1203, optional input devices such as keyboard 1215 and/or mouse 1216 and optional monitor 1207. Data communication can be achieved through the indicated communication medium to a server at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present disclosure can be transmitted over such networks or connections for reception and/or review by a party 1222 as illustrated in FIG. 16.

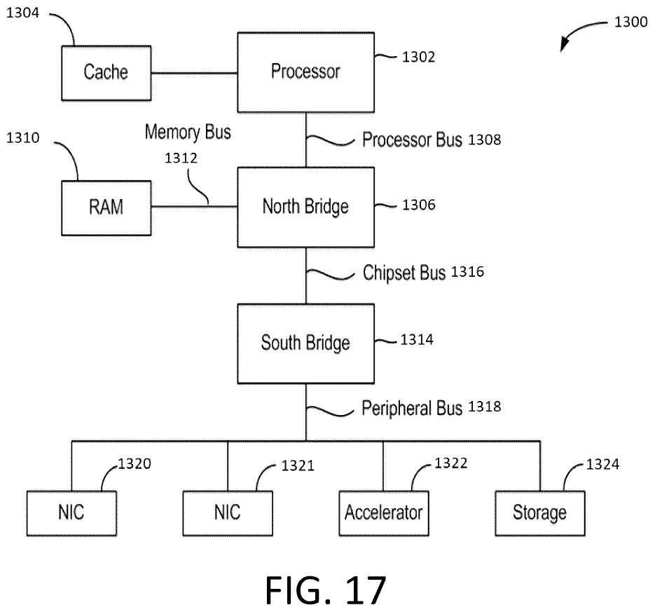

FIG. 17 is a block diagram illustrating a first example architecture of a computer system 1300 that can be used in connection with example instances of the present disclosure. As depicted in FIG. 17, the example computer system can include a processor 1302 for processing instructions. Non-limiting examples of processors include: Intel Xeon™ processor, AMD Opteron™ processor, Samsung 32-bit RISC ARM 1176JZ(F)-S v1.0™ processor, ARM Cortex-A8 Samsung S5PC100™ processor, ARM Cortex-A8 Apple A4™ processor, Marvell PXA 930™ processor, or a functionally-equivalent processor. Multiple threads of execution can be used for parallel processing. In some instances, multiple processors or processors with multiple cores can also be used, whether in a single computer system, in a cluster, or distributed across systems over a network comprising a plurality of computers, cell phones, and/or personal data assistant devices.

As illustrated in FIG. 17, a high speed cache 1304 can be connected to, or incorporated in, the processor 1302 to provide a high speed memory for instructions or data that have been recently, or are frequently, used by processor 1302. The processor 1302 is connected to a north bridge 1306 by a processor bus 1308. The north bridge 1306 is connected to random access memory (RAM) 1310 by a memory bus 1312 and manages access to the RAM 1310 by the processor 1302. The north bridge 1306 is also connected to a south bridge 1314 by a chipset bus 1316. The south bridge 1314 is, in turn, connected to a peripheral bus 1318. The peripheral bus can be, for example, PCI, PCI-X, PCI Express, or other peripheral bus. The north bridge and south bridge are often referred to as a processor chipset and manage data transfer between the processor, RAM, and peripheral components on the peripheral bus 1318. In some alternative architectures, the functionality of the north bridge can be incorporated into the processor instead of using a separate north bridge chip. In some instances, system 1300 can include an accelerator card 1322 attached to the peripheral bus 1318. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 1324 and can be loaded into RAM 1310 and/or cache 1304 for use by the processor. The system 1300 includes an operating system for managing system resources; non-limiting examples of operating systems include: Linux, Windows™, MACOS™, BlackBerry OS™ iOS™, and other functionally-equivalent operating systems, as well as application software running on top of the operating system for managing data storage and optimization in accordance with example instances of the present disclosure. In this example, system 1300 also includes network interface cards (NICs) 1320 and 1321 connected to the peripheral bus for providing network interfaces to external storage, such as Network Attached Storage (NAS) and other computer systems that can be used for distributed parallel processing.

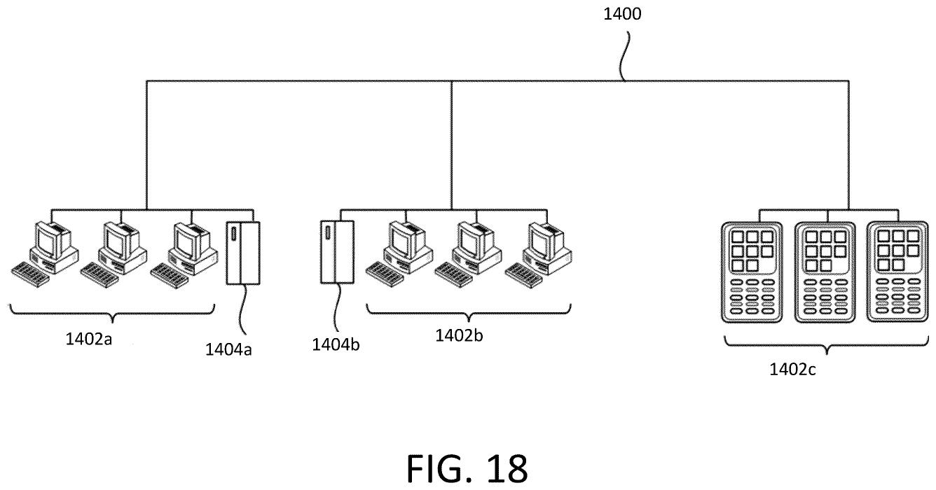

FIG. 18 is a diagram showing a network 1400 with a plurality of computer systems 1402a, and 1402b, a plurality of cell phones and personal data assistants 1402c, and Network Attached Storage (NAS) 1404a, and 1404b. In example instances, systems 1402a, 1402b, and 1402c can manage data storage and optimize data access for data stored in Network Attached Storage (NAS) 1404a and 1404b. A mathematical model can be used for the data and be evaluated using distributed parallel processing across computer systems 1402a, and 1402b, and cell phone and personal data assistant systems 1402c. Computer systems 1402a, and 1402b, and cell phone and personal data assistant systems 1402c can also provide parallel processing for adaptive data restructuring of the data stored in Network Attached Storage (NAS) 1404a and 1404b. FIG. 18 illustrates an example only, and a wide variety of other computer architectures and systems can be used in conjunction with the various instances of the present disclosure. For example, a blade server can be used to provide parallel processing. Processor blades can be connected through a back plane to provide parallel processing. Storage can also be connected to the back plane or as Network Attached Storage (NAS) through a separate network interface. In some example instances, processors can maintain separate memory spaces and transmit data through network interfaces, back plane or other connectors for parallel processing by other processors. In other instances, some or all of the processors can use a shared virtual address memory space.

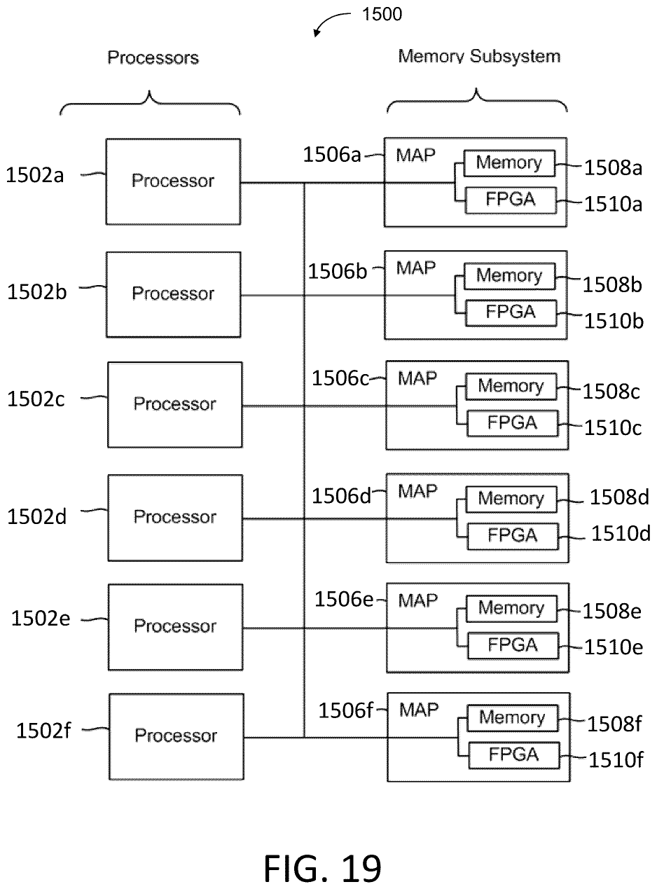

FIG. 19 is a block diagram of a multiprocessor computer system 1500 using a shared virtual address memory space in accordance with an example instance. The system includes a plurality of processors 1502a-f that can access a shared memory subsystem 1504. The system incorporates a plurality of programmable hardware memory algorithm processors (MAPs) 1506a-f in the memory subsystem 1504. Each MAP 1506a-f can comprise a memory 1508a-f and one or more field programmable gate arrays (FPGAs) 1510a-f. The MAP provides a configurable functional unit and particular algorithms or portions of algorithms can be provided to the FPGAs 1510a-f for processing in close coordination with a respective processor. For example, the MAPs can be used to evaluate algebraic expressions regarding the data model and to perform adaptive data restructuring in example instances. In this example, each MAP is globally accessible by all of the processors for these purposes. In one configuration, each MAP can use Direct Memory Access (DMA) to access an associated memory 1508a-f, allowing it to execute tasks independently of, and asynchronously from the respective microprocessor 1502a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example instances, including systems using any combination of general processors, co-processors, FPGAs and other programmable logic devices, system on chips (SOCs), application specific integrated circuits (ASICs), and other processing and logic elements. In some instances, all or part of the computer system can be implemented in software or hardware. Any variety of data storage media can be used in connection with example instances, including random access memory, hard drives, flash memory, tape drives, disk arrays, Network Attached Storage (NAS) and other local or distributed data storage devices and systems.

In example instances, the computer system can be implemented using software modules executing on any of the above or other computer architectures and systems. In other instances, the functions of the system can be implemented partially or completely in firmware, programmable logic devices such as field programmable gate arrays (FPGAs) as referenced in FIG. 19, system on chips (SOCs), application specific integrated circuits (ASICs), or other processing and logic elements. For example, the Set Processor and Optimizer can be implemented with hardware acceleration through the use of a hardware accelerator card, such as accelerator card 1322 illustrated in FIG. 17.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Functionalization of a Substrate Surface

A substrate was functionalized to support the attachment and synthesis of a library of polynucleotides. The substrate surface was first wet cleaned using a piranha solution comprising 90% H₂SO₄ and 10% H₂O₂ for 20 minutes. The substrate was rinsed in several beakers with DI water, held under a DI water gooseneck faucet for 5 minutes, and dried with N₂. The substrate was subsequently soaked in NH₄OH (1:100; 3 mL:300 mL) for 5 minutes, rinsed with DI water using a handgun, soaked in three successive beakers with DI water for 1 minute each, and then rinsed again with DI water using the handgun. The substrate was then plasma cleaned by exposing the substrate surface to 02. A SAMCO PC-300 instrument was used to plasma etch 02 at 250 watts for 1 minute in downstream mode.

The cleaned substrate surface was actively functionalized with a solution comprising N-(3-triethoxysilylpropyl)-4-hydroxybutyramide using a YES-1224P vapor deposition oven system with the following parameters: 0.5 to 1 torr, 60 minutes, 70° C., 135° C. vaporizer. The substrate surface was resist coated using a Brewer Science 200× spin coater. SPR™ 3612 photoresist was spin coated on the substrate at 2500 rpm for 40 seconds. The substrate was pre-baked for 30 minutes at 90° C. on a Brewer hot plate. The substrate was subjected to photolithography using a Karl Suss MA6 mask aligner instrument. The substrate was exposed for 2.2 seconds and developed for 1 minute in MSF 26A. Remaining developer was rinsed with the handgun and the substrate soaked in water for 5 minutes. The substrate was baked for 30 minutes at 100° C. in the oven, followed by visual inspection for lithography defects using a Nikon L200. A descum process was used to remove residual resist using the SAMCO PC-300 instrument to O₂ plasma etch at 250 watts for 1 minute.

The substrate surface was passively functionalized with a 100 μL solution of perfluorooctyltrichlorosilane mixed with 10 μL light mineral oil. The substrate was placed in a chamber, pumped for 10 minutes, and then the valve was closed to the pump and left to stand for 10 minutes. The chamber was vented to air. The substrate was resist stripped by performing two soaks for 5 minutes in 500 mL NMP at 70° C. with ultrasonication at maximum power (9 on Crest system). The substrate was then soaked for 5 minutes in 500 mL isopropanol at room temperature with ultrasonication at maximum power. The substrate was dipped in 300 mL of 200 proof ethanol and blown dry with N₂. The functionalized surface was activated to serve as a support for polynucleotide synthesis.

### Example 2: Synthesis of a 50-Mer Sequence on a Polynucleotide Synthesis Device

A two dimensional polynucleotide synthesis device was assembled into a flowcell, which was connected to a flowcell (Applied Biosystems (AB1394 DNA Synthesizer”). The polynucleotide synthesis device was uniformly functionalized with N-(3-TRIETHOXYSILYLPROPYL)-4-HYDROXYBUTYRAMIDE (Gelest) was used to synthesize an exemplary polynucleotide of 50 bp (“50-mer polynucleotide”) using polynucleotide synthesis methods described herein.

The sequence of the 50-mer was as described in SEQ ID NO.: 1. 5′AGACAATCAACCATTTGGGGTGGACAGCCTTGACCTCTAGACTTCGGCAT##TTTTTT TTTT3′ (SEQ ID NO.:1), where #denotes Thymidine-succinyl hexamide CED phosphoramidite (CLP-2244 from ChemGenes), which is a cleavable linker enabling the release of polynucleotides from the surface during deprotection.

The synthesis was done using standard DNA synthesis chemistry (coupling, capping, oxidation, and deblocking) according to the protocol in Table 2 and an ABI synthesizer.

TABLE 2

| | Table 2 | |
|---|---|---|
| General DNA Synthesis | | Time |
| Process Name | Process Step | (seconds) |
| WASH (Acetonitrile Wash | Acetonitrile System Flush | 4 |
| Flow) | Acetonitrile to Flowcell | 23 |
| | N2 System Flush | 4 |
| | Acetonitrile System Flush | 4 |
| DNA BASE ADDITION | Activator Manifold Flush | 2 |
| (Phosphoramidite + | Activator to Flowcell | 6 |
| Activator Flow) | Activator + | 6 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Activator to Flowcell | 0.5 |
| | Activator + | 5 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| DNA BASE ADDITION | Activator Manifold Flush | 2 |
| (Phosphoramidite + | Activator to Flowcell | 5 |
| Activator Flow) | Activator + | 18 |
| | Phosphoramidite to | |
| | Flowcell | |
| | Incubate for 25 sec | 25 |
| WASH (Acetonitrile Wash | Acetonitrile System Flush | 4 |
| Flow) | Acetonitrile to Flowcell | 15 |
| | N2 System Flush | 4 |
| | Acetonitrile System Flush | 4 |
| CAPPING (CapA + B, 1:1, | CapA + B to Flowcell | 15 |
| Flow) | | |
| WASH (Acetonitrile Wash | Acetonitrile System Flush | 4 |
| Flow) | Acetonitrile to Flowcell | 15 |
| | Acetonitrile System Flush | 4 |
| OXIDATION (Oxidizer | Oxidizer to Flowcell | 18 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 15 |
| | Acetonitrile System Flush | 4 |
| | Acetonitrile to Flowcell | 15 |
| | N2 System Flush | 4 |
| | Acetonitrile System Flush | 4 |
| | Acetonitrile to Flowcell | 23 |
| | N2 System Flush | 4 |
| | Acetonitrile System Flush | 4 |
| DEBLOCKING (Deblock | Deblock to Flowcell | 36 |
| Flow) | | |
| WASH (Acetonitrile Wash | Acetonitrile System Flush | 4 |
| Flow) | N2 System Flush | 4 |
| | Acetonitrile System Flush | 4 |
| | Acetonitrile to Flowcell | 18 |
| | N2 System Flush | 4.13 |
| | Acetonitrile System Flush | 4.13 |
| | Acetonitrile to Flowcell | 15 |

The phosphoramidite/activator combination was delivered similar to the delivery of bulk reagents through the flowcell. No drying steps were performed as the environment stays “wet” with reagent the entire time.

The flow restrictor was removed from the ABI 394 synthesizer to enable faster flow. Without flow restrictor, flow rates for amidites (0.1M in ACN), Activator, (0.25M Benzoylthiotetrazole (“BTT”; 30-3070-xx from GlenResearch) in ACN), and Ox (0.02M 12 in 20% pyridine, 10% water, and 70% THF) were roughly ~100 uL/second, for acetonitrile (“ACN”) and capping reagents (1:1 mix of CapA and CapB, wherein CapA is acetic anhydride in THF/Pyridine and CapB is 16% 1-methylimidizole in THF), roughly ~200 uL/second, and for Deblock (3% dichloroacetic acid in toluene), roughly ~300 uL/second (compared to ~50 uL/second for all reagents with flow restrictor). The time to completely push out Oxidizer was observed, the timing for chemical flow times was adjusted accordingly and an extra ACN wash was introduced between different chemicals. After polynucleotide synthesis, the chip was deprotected in gaseous ammonia overnight at 75 psi. Five drops of water were applied to the surface to recover polynucleotides. The recovered polynucleotides were then analyzed on a BioAnalyzer small RNA chip (data not shown).

### Example 3: Synthesis of a 100-Mer Sequence on a Polynucleotide Synthesis Device

The same process as described in Example 2 for the synthesis of the 50-mer sequence was used for the synthesis of a 100-mer polynucleotide (“100-mer polynucleotide”; 5′ CGGGATCCTTATCGTCATCGTCGTACAGATCCCGACCCATTTGCTGTCCACCAGTCATG CTAGCCATACCATGATGATGATGATGATGAGAACCCCGCAT##TTTTTTTTTT3′, where #denotes Thymidine-succinyl hexamide CED phosphoramidite (CLP-2244 from ChemGenes); SEQ ID NO.: 2) on two different silicon chips, the first one uniformly functionalized with N-(3-TRIETHOXYSILYLPROPYL)-4-HYDROXYBUTYRAMIDE and the second one functionalized with 5/95 mix of 11-acetoxyundecyltriethoxysilane and n-decyltriethoxysilane, and the polynucleotides extracted from the surface were analyzed on a BioAnalyzer instrument (data not shown).

All ten samples from the two chips were further PCR amplified using a forward (5′ATGCGGGGTTCTCATCATC3′; SEQ ID NO.: 3) and a reverse (5′CGGGATCCTTATCGTCATCG3′; SEQ ID NO.: 4) primer in a 50 uL PCR mix (25 uL NEB Q5 master mix, 2.5 uL 10 uM Forward primer, 2.5 uL 10 uM Reverse primer, 1 uL polynucleotide extracted from the surface, and water up to 50 uL) using the following thermal cycling program:
98 C, 30 seconds
98 C, 10 seconds; 63C, 10 seconds; 72C, 10 seconds; repeat 12 cycles
72C, 2 minutes

The PCR products were also run on a BioAnalyzer (data not shown), demonstrating sharp peaks at the 100-mer position. Next, the PCR amplified samples were cloned, and Sanger sequenced. Table 3 summarizes the results from the Sanger sequencing for samples taken from spots 1-5 from chip 1 and for samples taken from spots 6-10 from chip 2.

TABLE 3

| | | |
|---|---|---|
| Spot | Error rate | Cycle efficiency |

| | | | |
|---|---|---|---|
| 1 | 1/763 | bp | 99.87% |
| 2 | 1/824 | bp | 99.88% |
| 3 | 1/780 | bp | 99.87% |
| 4 | 1/429 | bp | 99.77% |
| 5 | 1/1525 | bp | 99.93% |
| 6 | 1/1615 | bp | 99.94% |
| 7 | 1/531 | bp | 99.81% |
| 8 | 1/1769 | bp | 99.94% |
| 9 | 1/854 | bp | 99.88% |
| 10 | 1/1451 | bp | 99.93% |

Thus, the high quality and uniformity of the synthesized polynucleotides were repeated on two chips with different surface chemistries. Overall, 89%, corresponding to 233 out of 262 of the 100-mers that were sequenced were perfect sequences with no errors.

Finally, Table 4 summarizes error characteristics for the sequences obtained from the polynucleotides samples from spots 1-10.

TABLE 4

| | Sample ID/Spot no. | | | | | | | | | |
|---|---|---|---|---|---|---|---|---|---|---|
| | OSA_{—} | OSA_{—} | OSA_{—} | OSA_{—} | OSA_{—} | OSA_{—} | OSA_{—} | OSA_{—} | OSA_{—} | OSA_{—} |
| | 0046/1 | 0047/2 | 0048/3 | 0049/4 | 0050/5 | 0051/6 | 0052/7 | 0053/8 | 0054/9 | 0055/10 |
| Total | 32 | 32 | 32 | 32 | 32 | 32 | 32 | 32 | 32 | 32 |
| Sequences | | | | | | | | | | |
| Sequencing | 25 of 28 | 27 of 27 | 26 of 30 | 21 of 23 | 25 of 26 | 29 of 30 | 27 of 31 | 29 of 31 | 28 of 29 | 25 of 28 |
| Quality | | | | | | | | | | |
| Oligo | 23 of 25 | 25 of 27 | 22 of 26 | 18 of 21 | 24 of 25 | 25 of 29 | 22 of 27 | 28 of 29 | 26 of 28 | 20 of 25 |
| Quality | | | | | | | | | | |
| ROI | 2500 | 2698 | 2561 | 2122 | 2499 | 2666 | 2625 | 2899 | 2798 | 2348 |
| Match | | | | | | | | | | |
| Count | | | | | | | | | | |
| ROI | 2 | 2 | 1 | 3 | 1 | 0 | 2 | 1 | 2 | 1 |
| Mutation | | | | | | | | | | |
| ROI Multi | 10 | 0 | 0 | 0 | 0 | 0 | 0 | 0 | 0 | 0 |
| Base | | | | | | | | | | |
| Deletion | | | | | | | | | | |
| ROI Small | 1 | 0 | 0 | 0 | 0 | 0 | 0 | 0 | 0 | 0 |
| Insertion | | | | | | | | | | |
| ROI | 0 | 0 | 0 | 0 | 0 | 0 | 0 | 0 | 0 | 0 |
| Single | | | | | | | | | | |
| Base | | | | | | | | | | |
| Deletion | | | | | | | | | | |
| Large | 0 | 0 | 1 | 0 | 0 | 1 | 1 | 0 | 0 | 0 |
| Deletion | | | | | | | | | | |
| Count | | | | | | | | | | |
| Mutation: | 2 | 2 | 1 | 2 | 1 | 0 | 2 | 1 | 2 | 1 |
| G > A | | | | | | | | | | |
| Mutation: | 0 | 0 | 0 | 1 | 0 | 0 | 0 | 0 | 0 | 0 |
| T > C | | | | | | | | | | |
| ROI Error | 3 | 2 | 2 | 3 | 1 | 1 | 3 | 1 | 2 | 1 |
| Count | | | | | | | | | | |
| ROI Error | Err: ~1 | Err: ~1 | Err: ~1 | Err: ~1 | Err: ~1 | Err: ~1 | Err: ~1 | Err: ~1 | Err: ~1 | Err: ~1 |
| Rate | in 834 | in 1350 | in 1282 | in 708 | in 2500 | in 2667 | in 876 | in 2900 | in 1400 | in 2349 |
| ROI | MP | MP | MP | MP | MP | MP | MP | MP | MP | MP |
| Minus | Err: ~1 | Err: ~1 | Err: ~1 | Err: ~1 | Err: ~1 | Err: ~1 | Err: ~1 | Err: ~1 | Err: ~1 | Err: ~1 |
| Primer | in 763 | in 824 | in 780 | in 429 | in 1525 | in 1615 | in 531 | in 1769 | in 854 | in 1451 |
| Error Rate | | | | | | | | | | |

### Example 4: Parallel Assembly of 29,040 Unique Polynucleotides

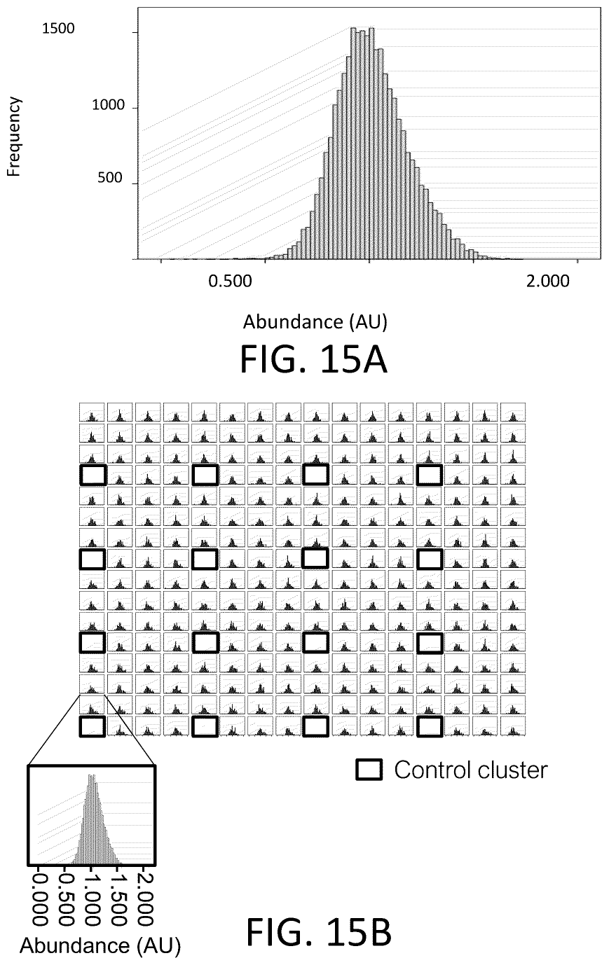

A structure comprising 256 clusters each comprising 121 loci on a flat silicon plate 1001 was manufactured as shown in FIG. 14. An expanded view of a cluster is shown in 1005 with 121 loci. Loci from 240 of the 256 clusters provided an attachment and support for the synthesis of polynucleotides having distinct sequences. Polynucleotide synthesis was performed by phosphoramidite chemistry using general methods from Example 3. Loci from 16 of the 256 clusters were control clusters. The global distribution of the 29,040 unique polynucleotides synthesized (240×121) is shown in FIG. 15A. Polynucleotide libraries were synthesized at high uniformity. 90% of sequences were present at signals within 4× of the mean, allowing for 100% representation. Distribution was measured for each cluster, as shown in FIG. 15B. On a global level, all polynucleotides in the run were present and 99% of the polynucleotides had abundance that was within 2× of the mean indicating synthesis uniformity. This same observation was consistent on a per-cluster level.

The error rate for each polynucleotide was determined using an Illumina MiSeq gene sequencer. The error rate distribution for the 29,040 unique polynucleotides averages around 1 in 500 bases, with some error rates as low as 1 in 800 bases. Distribution was measured for each cluster. The library of 29,040 unique polynucleotides was synthesized in less than 20 hours. Analysis of GC percentage versus polynucleotide representation across all of the 29,040 unique polynucleotides showed that synthesis was uniform despite GC content.

### Example 6. Library Preparation with Universal Adapters

Nucleic acid samples (50 ug) were prepared comprising either dual-index adapters or universal adapters. A ligation master mix is prepared from 20 μL of ligation buffer 10 μL of ligation mix (containing ligase), and 15 uL water. The nucleic acid sample was combined with the ligation mix and incubated at 20 deg C. at 15 minutes. The mixture was then combined with 80 μL of magnetic DNA purification beads, and vortexed, followed by 5 minutes of incubation at room temperature. The mixture was then set on a magnetic plate for 1 min. The beads were then washed with 80% ethanol, incubated for 1 min, and the ethanol wash discarded. The wash was repeated once. Then, beads were air-dried for 5-10 minutes, removed from the magnetic plate, and treated with 17 μL of water, 10 mM Tris-HCl pH 8, or buffer EB. The mixture was homogenized and incubated 2 min at room temperature. The mixture was then placed again on the magnetic plate and incubated 3 min at room temperature, followed by removal of the supernatant containing the universal adapter-ligated genomic DNA. The universal-ligated genomic DNA is combined with 10 μL of barcoded primers and 25 μL of KAPA HiFi HotStart ReadyMix to attach barcodes to the universal primers. The following PCR conditions were used: 1) initialization at 98 deg C. for 45 seconds, 2) a second step comprising: a) denaturation at 98 deg C. for 15 sec, b) annealing at 60 deg C. for 30 sec, and c) extension at 72 deg C. for 30 sec; wherein second step is repeated for 6-8 cycles, 3) final extension at 72 deg C. for 1 minute, and 4) final hold at 4 deg C. Products were purified by DNA beads in a similar manner as previously described. The amplified barcoded library was analyzed on a Qubit dsDNA broad range quantification assay instrument. This library was then sequenced directly. Use of universal adapters resulted in increased library nucleic acid concentration after amplification relative to standard dual-index Y-adapters. The protocol utilizing universal adapters also led to higher total yields after amplification and lower adapter dimer formation. Additionally, a library prepared with universal adapters provided for lower AT dropouts compared to standard dual-index Y-adapters, and resulted in uniform representation of all index sequences. Similarly, universal adapters comprising 10 bp dual indices were utilized (8 PCR cycles, N=12). For comparison, standard full-length Y adapters were also tested for the same genomic DNA sample (10 PCR cycles, N=12).

### Example 7. Library Preparation with Universal Adapters and Enrichment

A nucleic acid sample was prepared using the general methods of Example 6, with modification: dual-index adapters were replaced with universal adapters. After ligation of universal adapters, amplification of the adapter-ligated sample nucleic acid library was conducted with a barcoded primer library, to generate a barcoded adapter-ligated sample nucleic acid library. This library was then subjected to analogous enrichment, purification, and sequencing steps. Use of universal adapters resulted in comparable or better sequencing outcomes.

### Example 8. General Synthesis of a Synthetic Cot-1 Library

A sample of cot-1 (derived from human placental DNA) was obtained from a commercial source, and sequenced via Next Generation Sequencing using established methods. The sequencing data was then mapped to bisulfite converted human genomes used previously to design methylation panels. All exome and refseq related targets were subtracted, and a bed file was generated from the bisulfite-converted human genome. The remaining targets were clustered, synthesized (with addition of universal primer flanking regions), amplified, and purified to generate a synthetic cot-1 library. Resulting polynucleotides in the cot-1 library were 120 bases in length.

### Example 9. Synthesis of a Synthetic Cot-1 Library Using k-Mers

Sequences to be blocked in the input genome were determined (e.g., repetitive, low complexity, or specific types of sequences) by counting the number of copies k-mers of a given size along the input genome (e.g., for bisulfite-like conversion in methylation applications the input genome constitutes two copies of the genome, each with C->T, or G->A mutations throughout as would result from bisulfite conversion of the unmethylated genome after amplification). K-mers are oligonucleotide sequences of a given length in the genome. The number of instances of k-mers allowing modifications (see below) are currently computed for all sequences 30 nt of length found within the input genome. K-mers were also computed to enable collapsing k-mers that differ by one or more mutations into a single “k-mer” entity for which all counts are added together, and/or to include counts for k-mers different or varying size.

K-mers were then filtered for those with at least N=a given number of copies in the input genome. N was set to 200, but in other instances is tuned or includes different numbers of copies, or various different k-mer sizes depending on application (e.g., lower copy numbers for large regions that still yield off-target at values of N<200, e.g., N=2 or higher). Filtering enables tuning a desired stringency and/or total sequences manufactured. K-mers were also clustered using a variety sequence clustering algorithms to enable blocking a similar target set with a reduced number of k-mers.

K-mers were then mapped back to the genome to recover the original positions of members of the k-mer entity in the genome. Different instances include different values for parameters, such as for example tolerance for mismatches (difference of 0 or more mutations in the genome sequence relative to the k-mer), size, similarity and membership to each kmer entity or mapping to genome, or other criteria that reduce or generalize the specificity to determined sequences.

Polynucleotides of a given length for the synthetic cot-1 library (120 bases in length) to be synthesized were designed, capturing the sequence centered the middle of the original k-mer location using the input genome(s). In some instances, this was adjusted by varying the size or mix of sizes of oligonucleotides synthesized which can modulate the strength, or the uniformity of the effect for different type of sequences. Additional steps in some instances included clustering or additionally filtering sequences to reduce number of targets, improve balancing of effect across all or subsets of the sources of off-target sequences, different nucleotide content across sequences, or other metrics of sequence composition and context which vary across the original population of detected k-mers or their relation to each other.

Polynucleotides were synthesized as described using the general procedures of Example 1 to generate the synthetic cot-1 library. Oligo sequences were binned by oligo GC content and printed in clusters. Clusters were amplified separately, then pooled together by PCR plate and purified. Purified product from each plate was then blended together at equal mass. Additional modifications to polynucleotides include in silico and in vitro changes such as splitting and/or tuning the concentration of kmers with different copy numbers (by binning all kmers by their frequency of representation in the genome and altering the concentration of bins to capture the variation in their representation).

### Example 10. Methylome Enrichment with Synthetic Cot-1

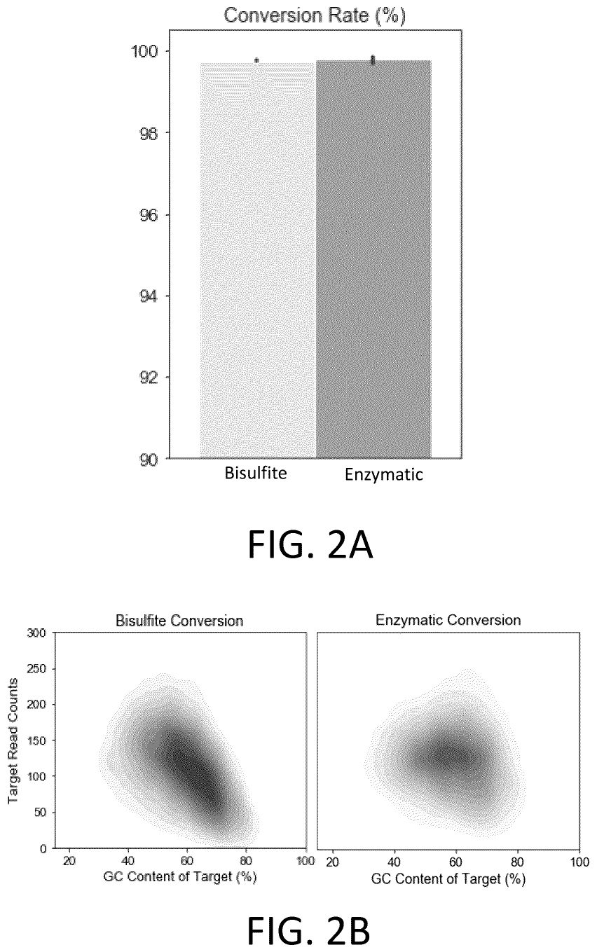

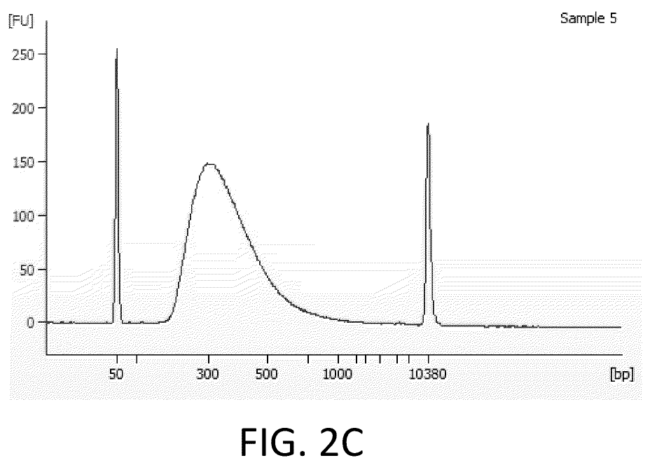

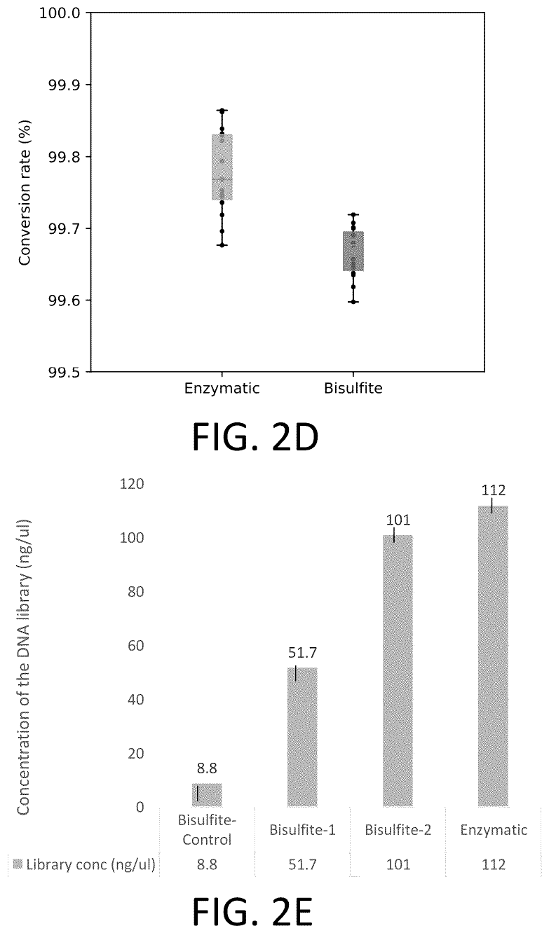

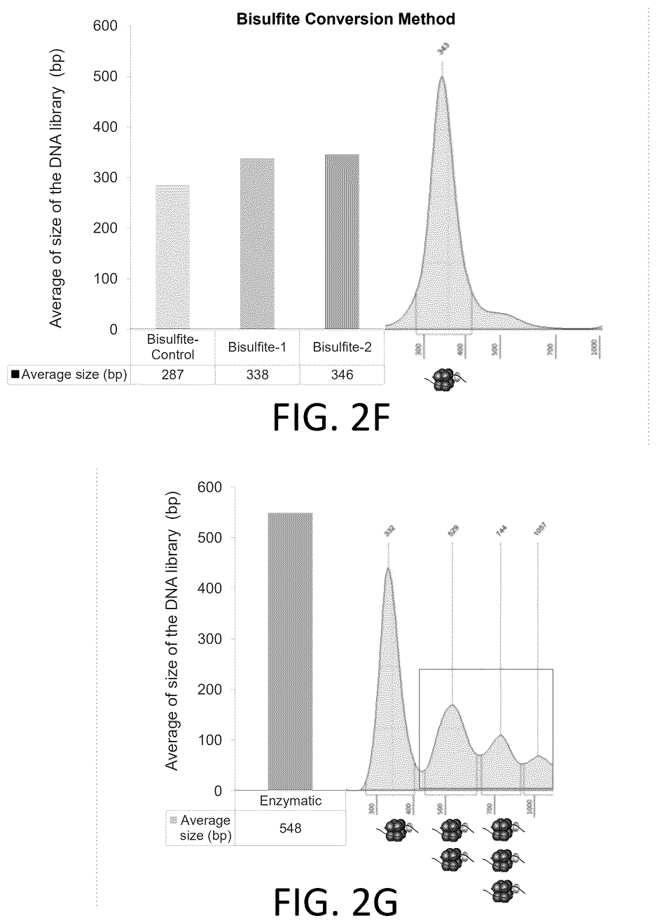

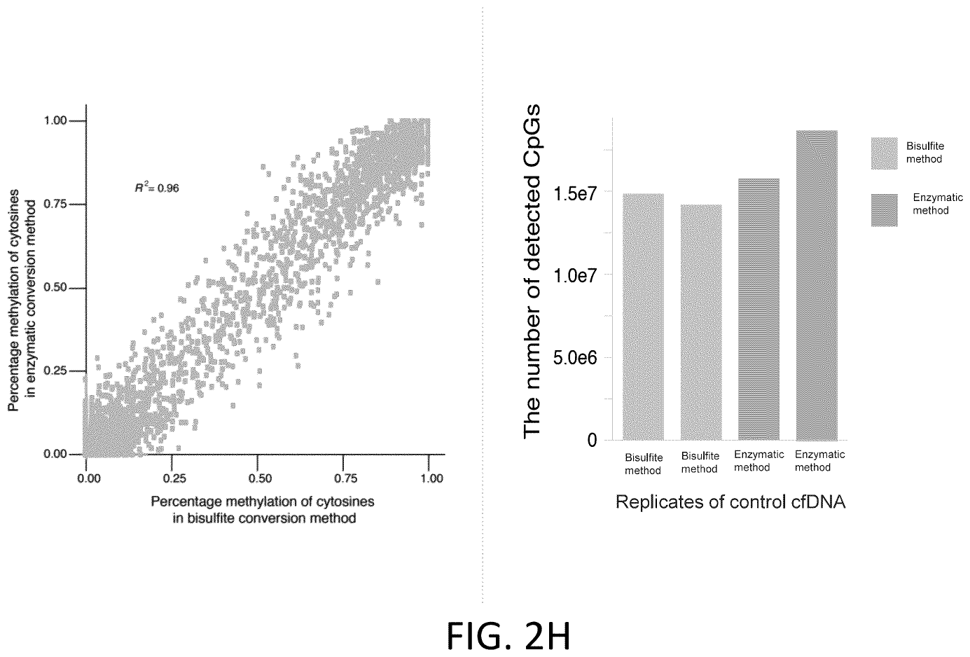

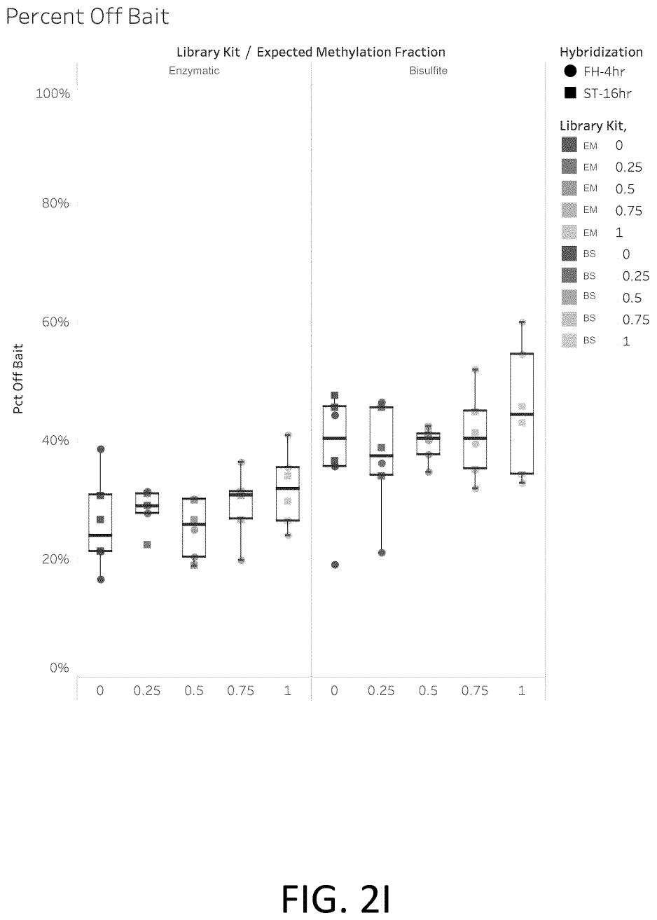

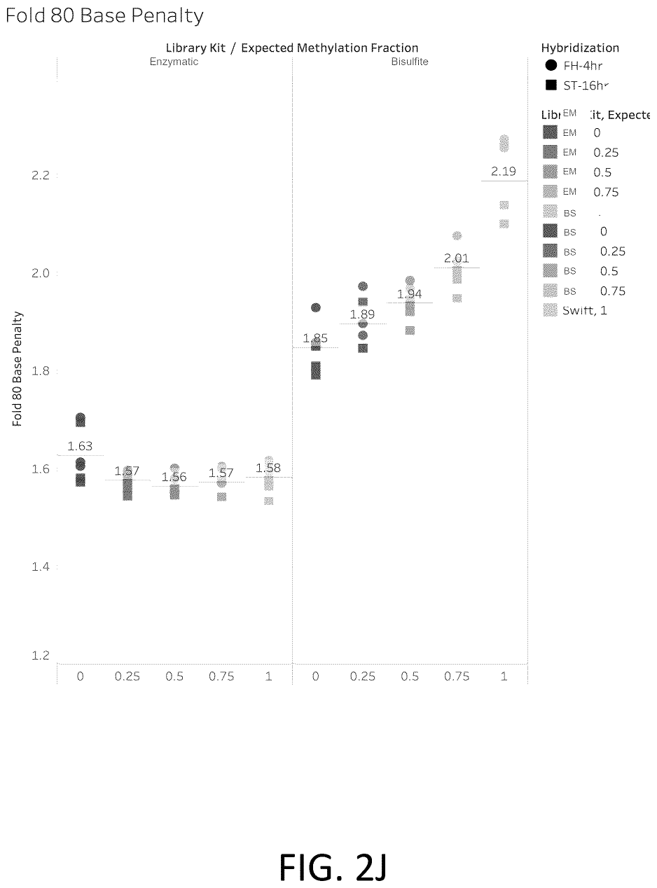

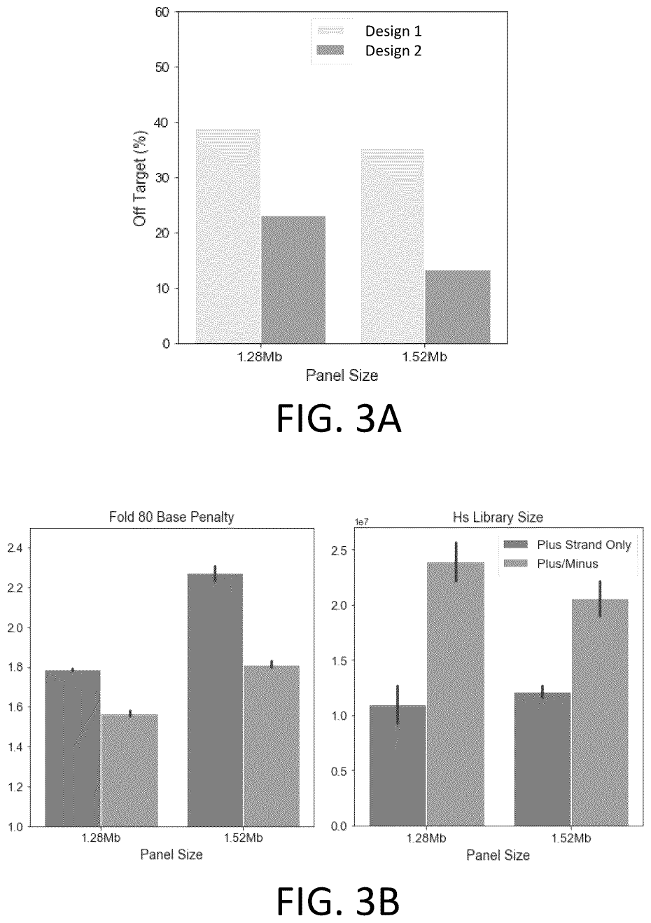

A sample comprising the NA12878 genome (Coriell) was prepared for methylation analysis using an enzymatic conversion of non-methylated cytosine to thymine (via uracil) following the manufacturer's instructions. Alternatively, the sample was treated with a bisulfite reagent to effect a similar transformation (FIG. 2A). Following the general procedure of Example 6 with modification, this sample was subjected to capture with a methylome-specific probe panel and employed a synthetic blocking library as prepared using the general methods of Example 9. Coverage of target GC content for each conversion method is shown in in FIG. 2B. Two different blocking library designs were tested, with design 2 showing improved off-target metrics (FIG. 3A). Additionally, blocking libraries targeting both + and − strands (and each with or without a putative C->T conversion) showed improved fold-80 and HS_library_size metrics relative to blocking libraries targeting only one strand (FIG. 3B) for two different capture panels tested (1.28 Mb and 1.52 Mb panels).

### Example 11: Fast Hybridization Buffers with Synthetic Blocking Library

Sequencing data was acquired using the general method of Example 6 and Example 10, with modification: the temperature of wash buffer 1 was varied to modify sequencing results, and the protocol was carried out as described below using 3 different methylome panels (0.04 Mb, 1.28 Mb, or 3.00 Mb).
Step 1. Adapter-ligated samples (generated from universal adapters), were transferred to a 0.2-ml thin-walled PCR strip-tube or 96-well plate. The methylome capture probe panel, universal blockers, and blocker solution/buffer, a non-polar hybridization enhancer, and the synthetic blocking library was added, the mixture pulse-spun, and the mixture evaporated using low or no heat.
Step 2. A 96-well thermal cycler was programmed with the following conditions and the heated lid set to 85° C., as shown in Table 5.

TABLE 5

| Step | Temperature | Time |
|---|---|---|
| 1 | 95° C. | HOLD |
| 2 | 95° C. | 5 minutes |
| 3 | Hybridization | HOLD |
| | temperature | |
| | (e.g., 60° C.) | |

The dried hybridization reactions were each resuspended in 20 μl fast hybridization buffer, and mixed by flicking. The tubes were pulse spun to minimize bubbles. 30 μl of liquid polymer was then added to the top of the hybridization reaction, and the tube pulse-spun. Tubes were transferred to the preheated thermal cycler and moved to Step 2 of the thermocycler program (incubate at 95° C. for 5 minutes). The tubes were then incubated at 60° C. for a time of 15 minutes to 4 hours in a thermal cycler with the lid at 85° C. 450 μl wash buffer 1 was heated the desired temperature (e.g., 70° C., or other temperature depending on desired sequencing metrics) and 700 μl wash buffer 2 was heated to 48° C. Streptavidin Binding Beads were equilibrated to room temperature for at least 30 minutes and then vortexed until mixed. 100 μl Streptavidin Binding Beads were added to a 1.5-ml microcentrifuge tube. One tube was prepared for each hybridization reaction. 200 μl fast binding buffer was added to the tubes and mixed by pipetting. The tubes were placed on a magnetic stand for 1 minute, then removed and the clear supernatant discarded, without disturbing the bead pellet. The tube was then removed from the magnetic stand. The pellet was washed two more times for a total of three washes with the fast binding buffer. After removing the clear supernatant from the third wash, a final 200 μl fast binding buffer was added and the beads resuspended by vortexing until homogenized. The tubes of the hybridization reaction were mixed with the Streptavidin Binding Beads for 30 minutes at room temperature on a shaker, rocker, or rotator at a speed sufficient to keep the solution mixed.
Step 3. Tubes containing the hybridization reaction with Streptavidin Binding Beads were removed from the mixer and pulse-spun to ensure solution was at the bottom of the tubes, and the tubes were placed on a magnetic stand for 1 minute. The clear supernatant including the liquid polymer was removed and discarded with disturbing the pellet. The tubes were removed from the magnetic stand and 200 μl preheated fast wash buffer 1 was added, then mixed by pipetting. The tubes were incubated for 5 minutes at 70° C., and placed on a magnetic stand for 1 minute. The clear supernatant was removed and discarded without disturbing the bead pellet. The tubes were then removed from the magnetic stand and an additional 200 μl of preheated fast wash buffer 1 was added, followed by mixing and incubation 5 minutes at 70° C. The tubes were pulse-spun to ensure solution was at the bottom of the tubes. After the hybridization is complete, the thermal cycler lid was opened and the volume of each hybridization reaction including liquid polymer quickly transferred into a corresponding tube of washed Streptavidin Binding Beads, then mixed. The entire volume (~200 μl) was transferred into a new 1.5-ml microcentrifuge tube, one per hybridization reaction. The tubes were placed on a magnetic stand for 1 minute, followed by removal and discard of the clear supernatant. The tubes were removed from the magnetic stand and 200 μl of 48° C. wash buffer 2 was added, mixed by pipetting, and then pulse-spun to ensure the solution was at the bottom of the tubes. The tuber were then incubated for 5 minutes at 48° C., placed on a magnetic stand for 1 minute, and the clear supernatant removed and discarded with disturbing the pellet. The wash step was repeated two more times, for a total of three washes. After the final wash, a 10 μl pipette was used to remove traces of supernatant. Without allowing the pellet to dry, the tubes were removed from the magnetic stand and 45 μl of water added, mixed, and then incubated on ice (hereafter referred to as the Streptavidin Binding Bead slurry).
Step 4. A thermal cycler was programmed with the following conditions in Table 6, and the heated lid set to 105° C. 22.5 μl of the Streptavidin Binding Bead slurry was transferred to a 0.2-ml thin-walled PCR strip-tubes and kept on ice until ready for use in the next step. A PCR mixture was prepared by adding a PCR polymerase mastermix and adapter-specific primers to the tubes containing the Streptavidin Binding Bead slurry and mixed by pipetting. The tubes were pulse-spun, and transferred to the thermal cycler and start the cycling program.

TABLE 6
Thermocycler program for PCR library amplification.

| | | | | | | Number |
|---|---|---|---|---|---|---|
| Step | | Temperature | | Time | | of Cycles |
| 1 | Initialization | 98 | C. | 45 | seconds | 1 |
| 2 | Denaturation | 98 | C. | 15 | seconds | Varies |
| | Annealing | 60 | C. | 30 | seconds | |
| | Extension | 72 | C. | 30 | seconds | |
| 3 | Final | 72 | C. | 1 | minute | 1 |
| | Extension | | | | | |

| | | | | | |
|---|---|---|---|---|---|
| 4 | Final Hold | 4 | C. | HOLD | — |

| | |
|---|---|
| Custom Panel Size | Number of Cycles |

| | | |
|---|---|---|
| >100 | Mb | 5 |
| 500-100 | Mb | 7 |
| 10-500 | Mb | 8 |
| 1-10 | Mb | 9 |
| 500-1,000 | kb | 11 |
| 100-500 | kb | 13 |
| 50-100 | kb | 14 |
| <50 | kb | 15 |

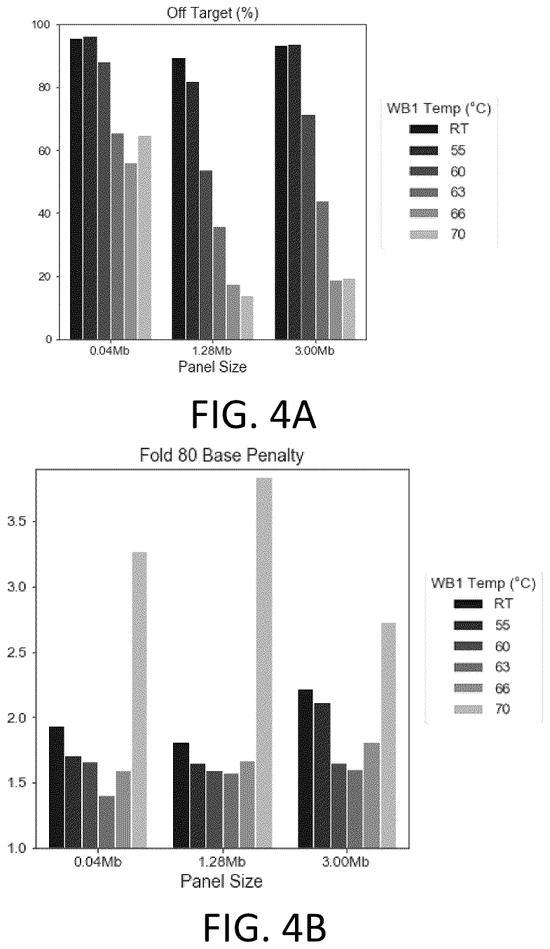

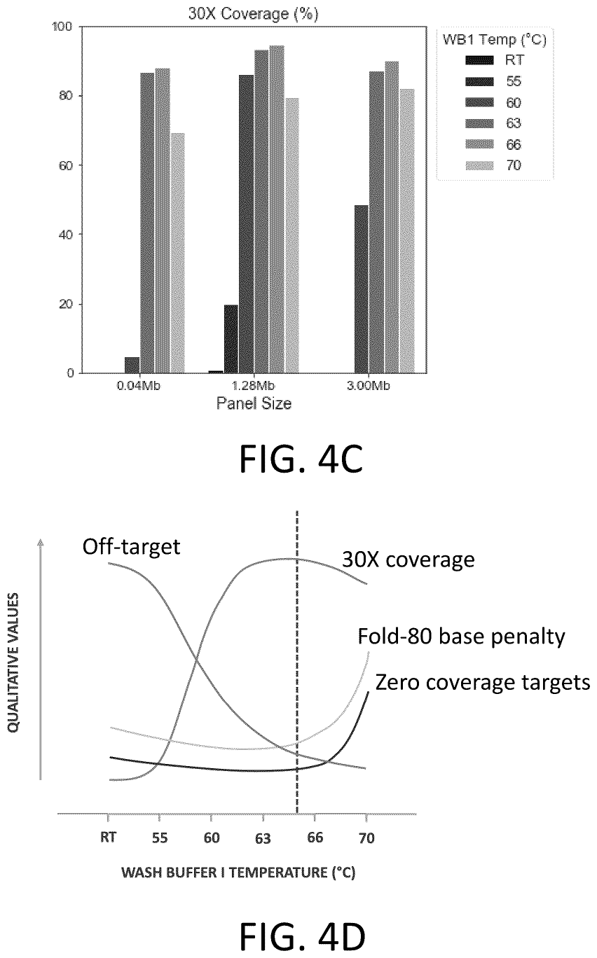

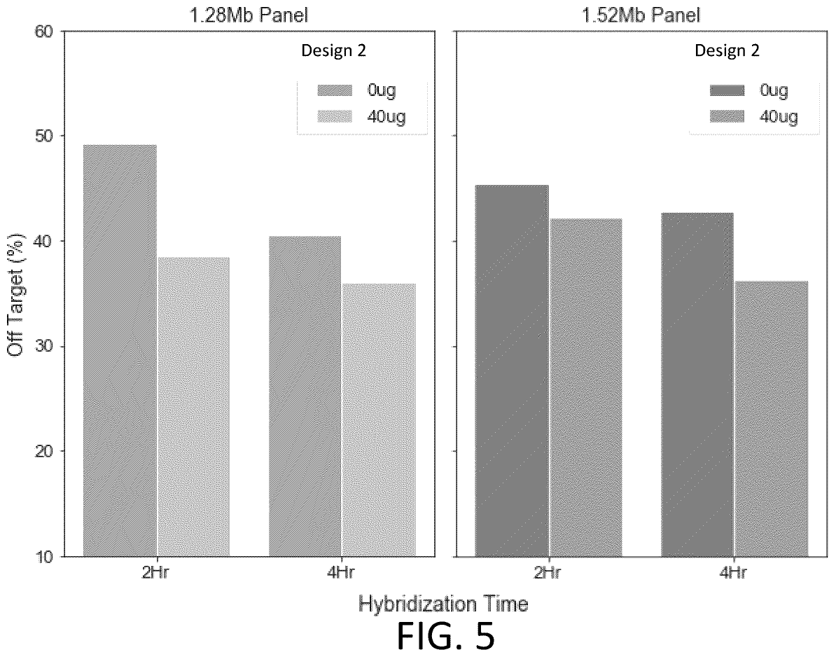

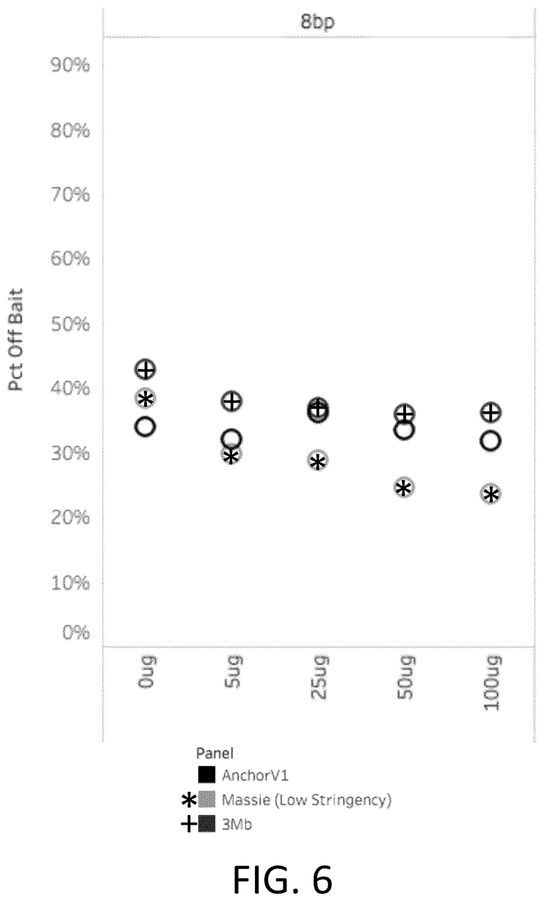

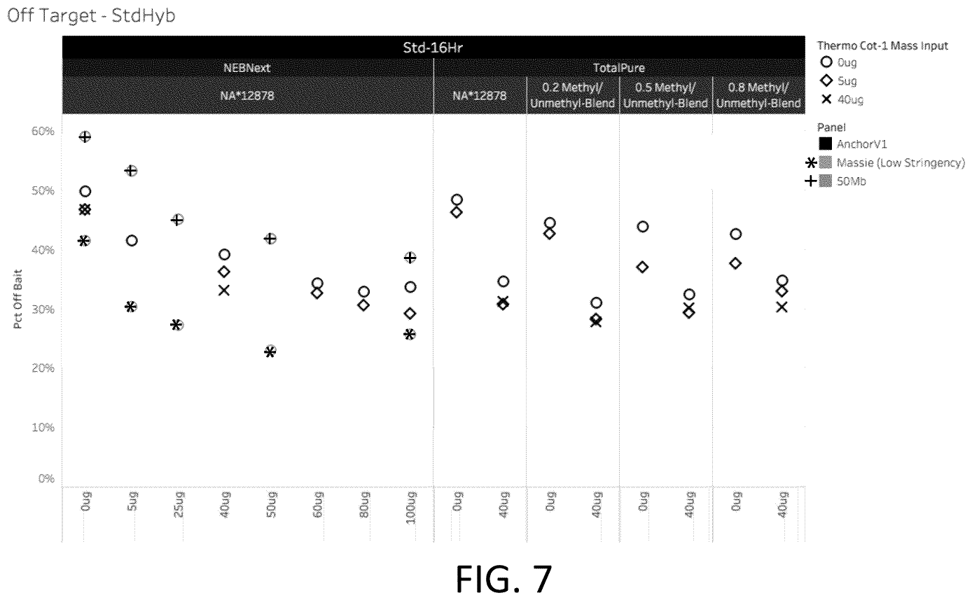

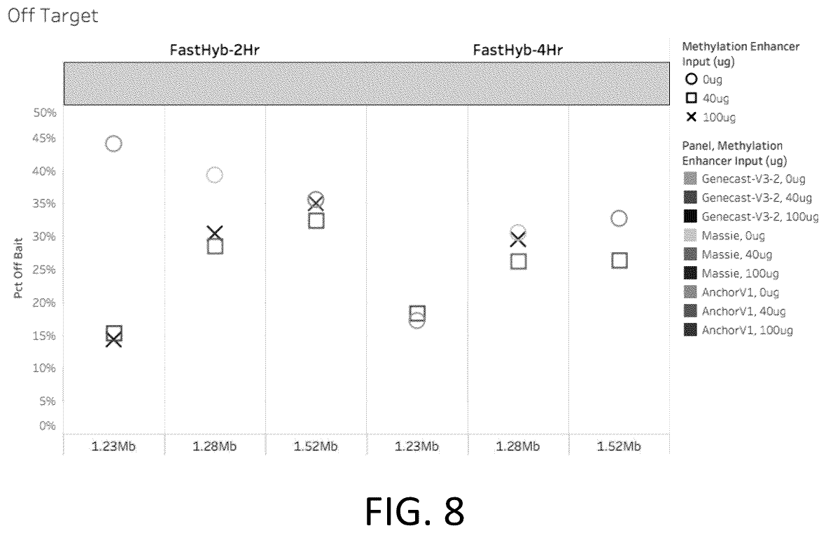

50 μl (1.0×) homogenized DNA Purification Beads were added to the tubes, mixed by vortexing, and incubated for 5 minutes at room temperature. The tubes were then placed on a magnetic plate for 1 minute. The clear supernatant was removed from the tubes. The DNA Purification Bead pellet was washed with 200 μl freshly prepared 80% ethanol for 1 minute, then the ethanol was removed and discarded. This wash was repeated once, for a total of two washes, while keeping the tube on the magnetic plate. A 10 μl pipet was used to remove residual ethanol, making sure to not disturb the bead pellet. The bead pellet was air-dried on a magnetic plate for 5-10 minutes or until the bead pellet was dry. The tubes were removed from the magnetic plate and 32 μl water was added. The resulting solution was mixed by pipetting until homogenized and incubated at room temperature for 2 minutes. The tubes were then placed on a magnetic plate and let stand for 3 minutes or until the beads fully pelleted. 30 μl of the clear supernatant containing the enriched library was transferred to a clean thin-walled PCR 0.2-ml strip-tube.
Step 5. Each enriched library was validated and quantified for size and quality using an appropriate assay, such as the Agilent BioAnalyzer High Sensitivity DNA Kit and a Thermo Fisher scientific Qubit dsDNA High Sensitivity Quantitation Assay. Samples were then loaded onto an Illumina sequencing instrument for analysis. Sampling was conducted at 250× (theoretical read depth), and mapping quality was >20. The effects on various NGS sequencing metrics for various fast hybridization wash buffer 1 temperatures are shown in FIG. 4A-4D. Results demonstrating the benefit of adding a synthetic blocking library using the fast hybridization system for two different hybridization times (2 hr and 4 hr) are show in FIG. 5. Further experiments were conducted to evaluate the amount of blocking library added, as well as use of the blocking reagent cot-1 for a series of NGS metrics. FIGS. 6-8. A summary of average workflow times for different steps is shown in Tables 7A-7B.

TABLE 7A
Library Preparation

| Protocol Step | Time | |
|---|---|---|
| Mechanical Fragmentation | 0.5 | hour |
| End Repair and A-tailing | 1 | hour |
| Adapter Ligation & Clean-up | 1 | hour |
| Oxidation & Clean-up | 2 | hours |
| Denaturation | 0.5 | hours |
| Deamination & Clean-up | 3.5 | hours |
| PCR & Clean-up | 1.5 | hour |
| Perform Library QC | 0.5 | hour |
| Total | ~10.5 | hours |

TABLE 7B
Target Enrichment

| Protocol Step | Time | |
|---|---|---|
| Prepare Libraries for Hybridization | 1 | hour |
| Hybridize Capture Probes with Pools | 0.5 hour plus flexible | |
| | 2 hours to 4 hours | |
| Bind Hybridized Targets to Streptavidin Beads | 1.5 | hours |
| Post-Capture PCR Amplification & Clean-up | 1 | hour |
| Perform Capture QC | 0.5 | hour |
| Pooling and/or Sample Sheet Generation | 0.5 | hour |
| Total | ~7 to 9 Hrs | |
| Sequencing using an Illumina | 32 | hours |
| NextSeq High Output Kit | | |

Total: ~39 to 41 hours (~7 to 9 hours of Capture Workflow and ~32 hours of Sequencing)

### Example 12. Evaluation of 1 Mb, 1.5 Mb, and 50 Mb Libraries

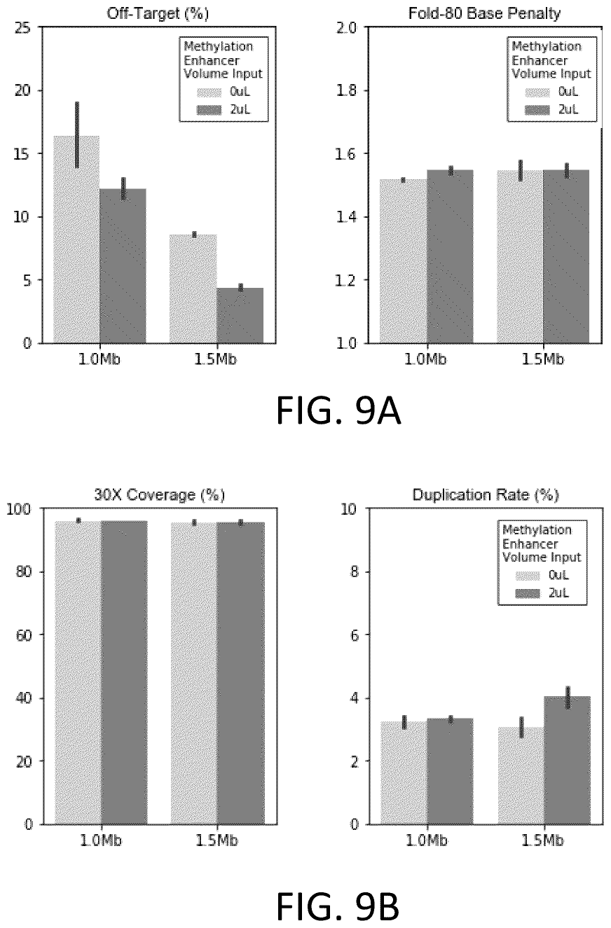

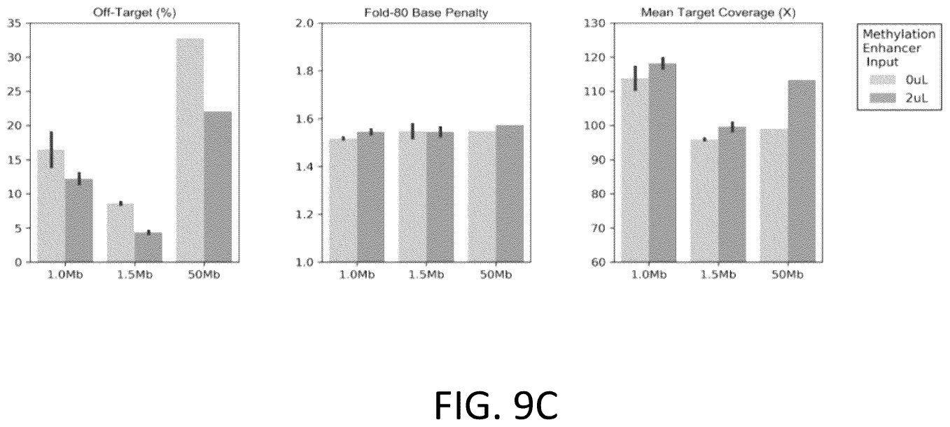

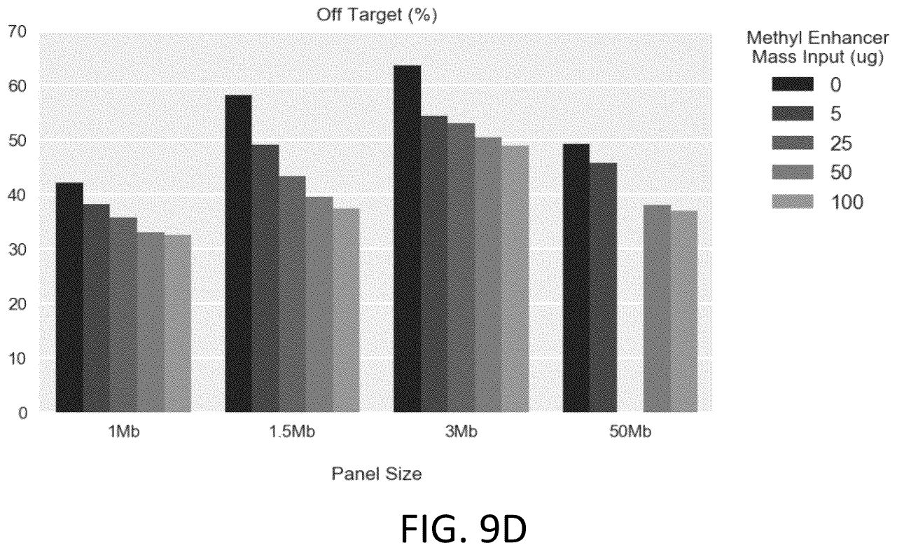

Following the general procedure of Example 11, 1.0 Mb and 1.5 Mb libraries were evaluated using the enzymatic conversion of unmethylated cytosines (EM-seq). EM-seq conversion involved a series of enzymatic steps to convert unmethylated cytosines into uracils. First, ten-eleven translocation dioxygenase 2 (TET2) and an Oxidation Enhancer converted methylated cytosines (5mC and 5hmC) to 5-carboxycytosine (5caC) and glucosylated 5hmC (5ghmC), respectively. This protected these cytosines from deamination by APOBEC in the next step, which occurred after denaturation. APOBEC deaminated unprotected (i.e. unmethylated) cytosines into uracils. Subsequent PCR amplification converted 5mC or 5hmC into cytosines and uracils into thymines. Results from hybridization in the presence or absence of methylation enhancer (design 2) are shown in FIGS. 9A-9B. Additionally, a larger 50 Mb library was tested using the same general workflow, and the results compared to a 1.0 Mb and 1.5 Mb library are shown in FIG. 9C. Additional amounts of enhancer were also tested in FIG. 9D.

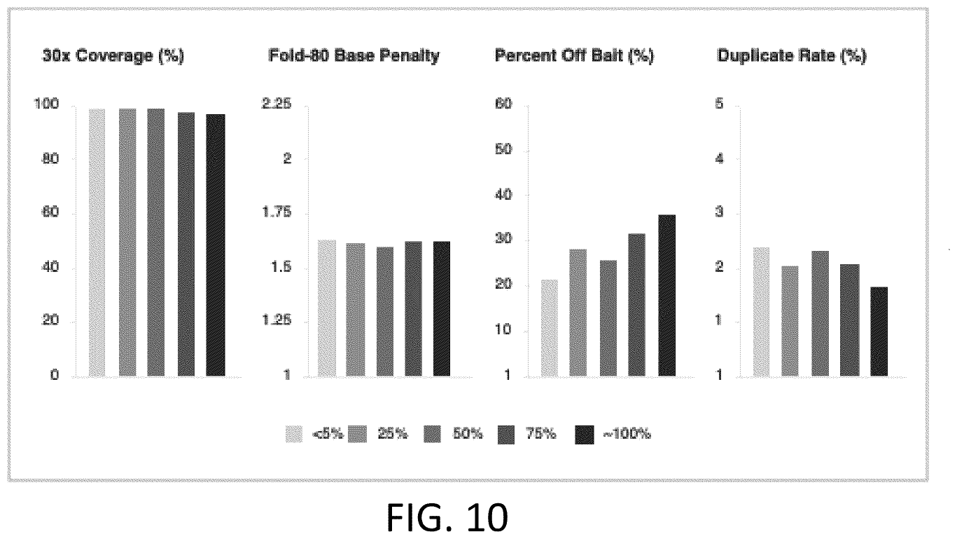

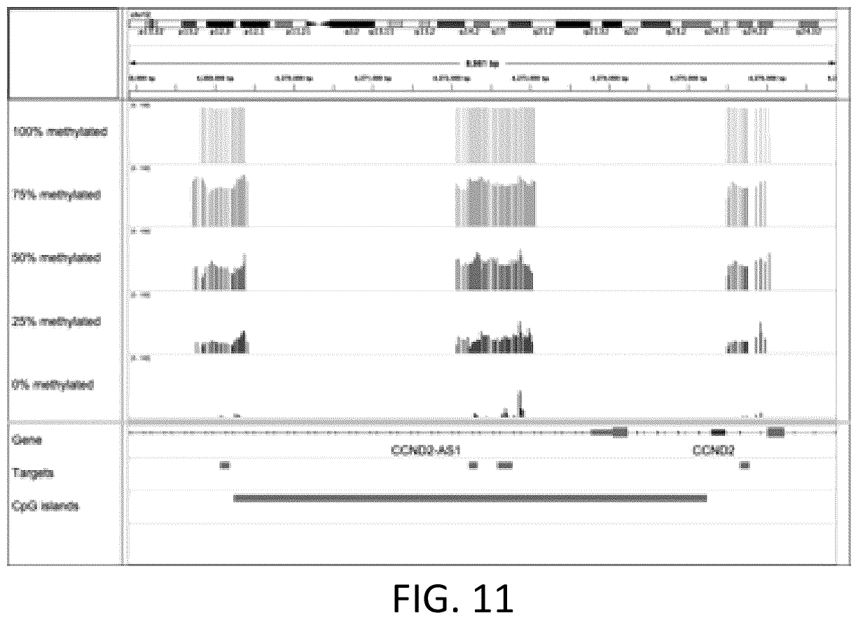

Methylation levels vary substantially across the human genome, and differentially methylated regions (DMRs) can be used to identify certain cancers. Libraries were prepared using the EM-seq conversion method and blends of hypo- and hypermethylated cell lines at ratios of 0, 25, 50, 75, and 100% methylation. A medium stringency designed 1 Mb panel was used to capture each gDNA library type. Sequencing was performed with a NextSeq® 500/550 High Output v2 kit to generate 2×151 paired end reads. Data was down-sampled to 250× aligned coverage relative to the panel target size, mapped using the Bismark Aligner, and analyzed using Picard Metrics with a mapping quality threshold of 20. Key hybrid selection metrics are steady for each gDNA library type, despite differences in CpG methylation levels. The effect of methylation level on the performance with libraries of varying methylation levels (0-100% methylation) were generated by combining hypo- and hypermethylated genomic DNA in defined ratios. This analysis showed minimal effects of methylation level on final sequencing metrics (FIG. 10).

### Example 13. Iterative Enhancement of Synthetic Cot-1

After the general procedure of Example 11 is conducted, the synthetic cot-1 library is further refined by using data from the capture to examine sequences that are still captured outside of desired target regions by: a) Using experimental results to determine regions that are on and off-target after alignment of sequencing reads to the input genome (e.g., in the case of bisulfite converted samples using methylation aware alignment software); b) Using off-target sequences to generate additional synthetic blocking oligos, optionally preceded or followed by clustering to reduce sequences; and/or c) Synthesizing and using the additional blockers synthesized in b) together with the original set of blockers, or alone if the experiment in is run without synthetic blockers; Optionally repeating this procedure one or more times to iteratively supplement, refine, and achieve additional enhancement.

### Example 14. Addition of Control DNAs

The general procedure of Example 11 was followed with modification: a control protocol was added to confirm conversion rates using DNA control of known methylation levels. CpG Methylated pUC19 DNA and Unmethylated Lambda DNA were used as methylation controls. Both controls possess known levels of methylation, enabling an accurate determination of the conversion rate post-sequencing. Because these controls may lack complementary probes in target enrichment panels, the controls were subjected to hybrid capture; instead, they were be stored until after hybrid capture and subsequently pooled with samples for sequencing.

To demonstrate the use of these controls, libraries were generated using the general protocol of Example 11. Forty-eight microliters of each DNA control were combined together in a single reaction, and the mix was dried down using a speed vacuum concentrator. The resulting dried DNA was resuspended in 50 μl of 0.1× TE pH 8.0 and moved through the library process.

Table 8 shows the measured versus expected conversion efficiency and post-sequencing methylation level. EM-seq met the expected efficiency at higher than 99.5% conversion for both controls. The expected CpG methylation levels of the Unmethylated Lambda DNA and CpG Methylated pUC19 DNA controls are 0.5% and 95-98%, respectively. The measured CpG methylation levels matched the expected levels; in the methylated control, 166 out of 177 CpG sites were methylated. These data indicate that DNA controls of known methylation levels can be used to ensure that the conversion process is complete and the assay's false positive is minimized. Conversion efficiency and CpG methylation level results when converting the CpG Methylated pUC19 DNA and Unmethylated Lambda DNA controls with EM-seq (Table 8).

TABLE 8
Expected versus Measured Conversion Efficiency
and CpG Methylation Levels.

| | | CpG |
|---|---|---|
| | Unmethylated | Methylated |
| | Lambda | pUC19 |
| Metric | DNA | DNA |
| Expected Conversion Efficiency | >=99.5% | >=99.5% |
| Measured Conversion Efficiency | 99.77% | 99.57% |
| Expected CpG Methylation Level | Up to 0.5% | 95-98% |
| Measured CpG Methylation Level | 0.22228% | 95.7572% |

### Example 15. Target Region Size

The general procedure of Example 11 was followed, using panel libraries of varying sizes. Many factors related to custom target regions influence the final targeted sequencing metrics; optimization may be needed in some instances for best performance. These factors include but are not limited to high GC content in the target region and very small panel designs (<0.5 Mb), which are in some instances particularly sensitive to hybridization. The optimal trade-off between inclusiveness and off-target control in some instances depends on characteristics of the target region and the panel's intended application. During the panel design process for example, a researcher working with a medium sized panel and a low number of samples may prefer to keep certain probes, even if they require additional sequencing to balance increased off-target capture. By contrast, those working with a much smaller panel (where off-target capture increases the required sequencing relative to rest of the panel more quickly) or with very large numbers of samples (where modest increases in cost can quickly add up), may prefer to use more stringent design conditions to optimize cost.

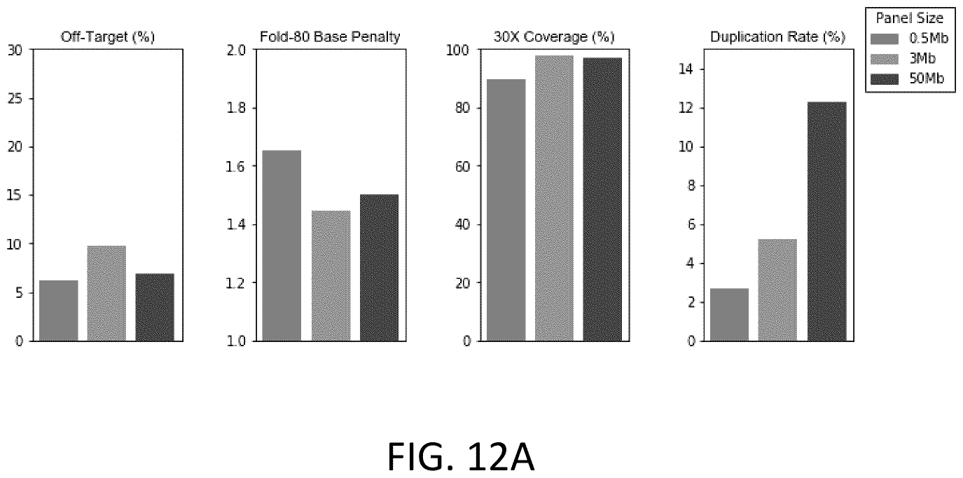

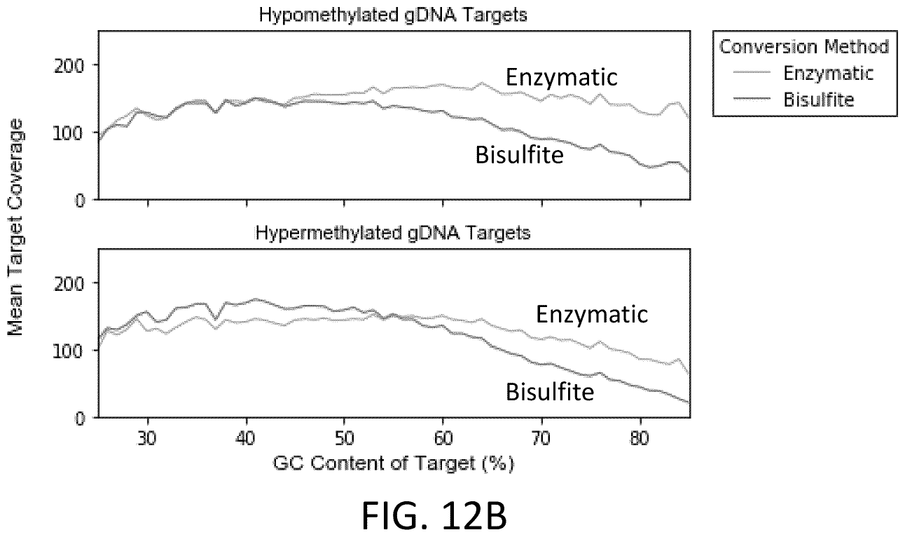

To evaluate the relationship between panel size and sequencing metrics, three different panels were used with the general procedures of Example 11. Together, the panels spanned a wide range of methylation targets and panel sizes: 0.5 Mb, 3 Mb, and 50 Mb. The largest panel used in this study provided off-target levels close to 7%, with all panels registering off-target levels under 10%. Capture uniformity (fold-80 base penalty) was exceptional for all target sizes, reaching values between 1.4 and 1.7. The proportion of probes with 30× coverage was higher than 90% for all panels. Capture metrics for methylation panels covering target sizes of 0.5 Mb, 3 Mb, and 50 Mb using the general protocols of Example 11 and a single-plex reaction are shown in FIG. 12A. Capture conditions, including 2 μl of Methylation Enhancer, a Wash Buffer 1 temperature of 65° C., and a 2-hour hybridization time were used in each reaction. Sequencing was performed with a NextSeq® 500/550 High Output v2 kit to generate 2×76 paired end reads. Data was down-sampled to 200× aligned coverage relative to the panel target size, mapped using the Bismark Aligner, and analyzed using Picard Metrics with a mapping quality threshold of 20.

### Example 16. Methylation Levels Differ Across the Genome

Because differential methylation levels can be used for early detection of specific cancers, it is advantageous for protocols used to detect methylation are highly compatible with custom panel designs and are capable of identifying hyper- and hypomethylated regions. Conversion leads to a decrease in sequence complexity, which can cause in some instances issues downstream in the hybrid capture step. However, these issues can be mitigated with library preparation reagents, hybrid capture reagents, and custom panel design, resulting in probe coverage that is evenly distributed across regions with varying AT/GC content and methylation levels.

Probe coverage plots were generated using a 1.5 Mb custom panel with hyper- and hypomethylated genomic DNA input material using two different conversion systems: EM-seq and bisulfite treatment. FIG. 12B shows an even distribution of target read counts for both methylation levels using the EM-seq conversion method (teal). By contrast, an industry-leading bisulfite conversion process (grey) resulted in comparatively uneven target read counts. The protocol was performed using a custom 1.5 Mb panel and a single-plex reaction. Capture conditions included 2 μl of Methylation Enhancer (design 2), a Wash Buffer 1 temperature of 65° C., and a 2-hour hybridization time were used in each reaction. Sequencing was performed with a NextSeq® 500/550 High Output v2 kit to generate 2×151 paired end reads. Data was down-sampled to 250× aligned coverage relative to the panel target size, mapped using the Bismark Aligner, and analyzed using Picard Metrics with a mapping quality threshold of 20. For both hypo- and hypermethylated gDNA types, target coverage is more evenly distributed across all GC bins when using enzymatic library preparation approach.

### Example 17. 123 Mb Methylome Panel

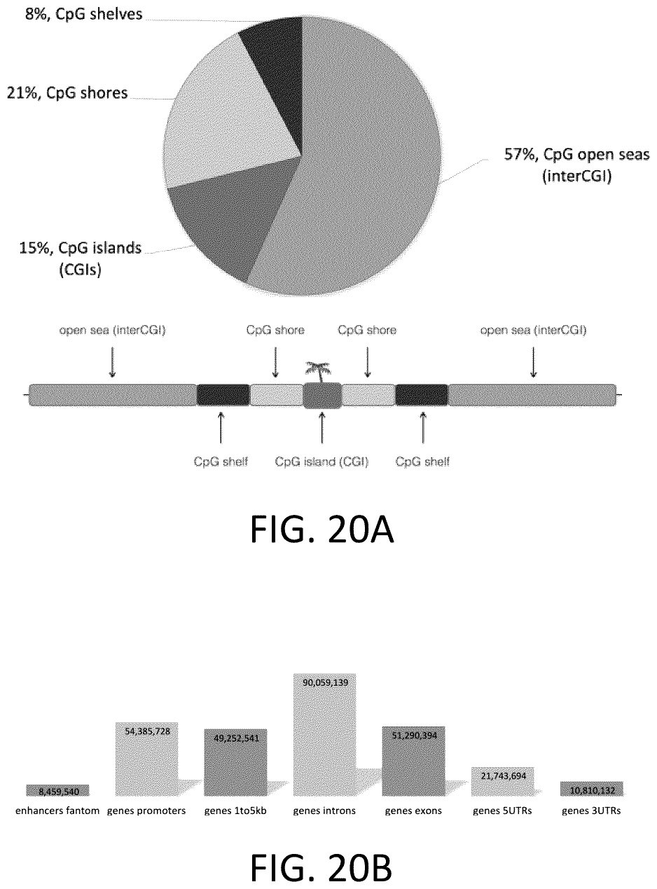

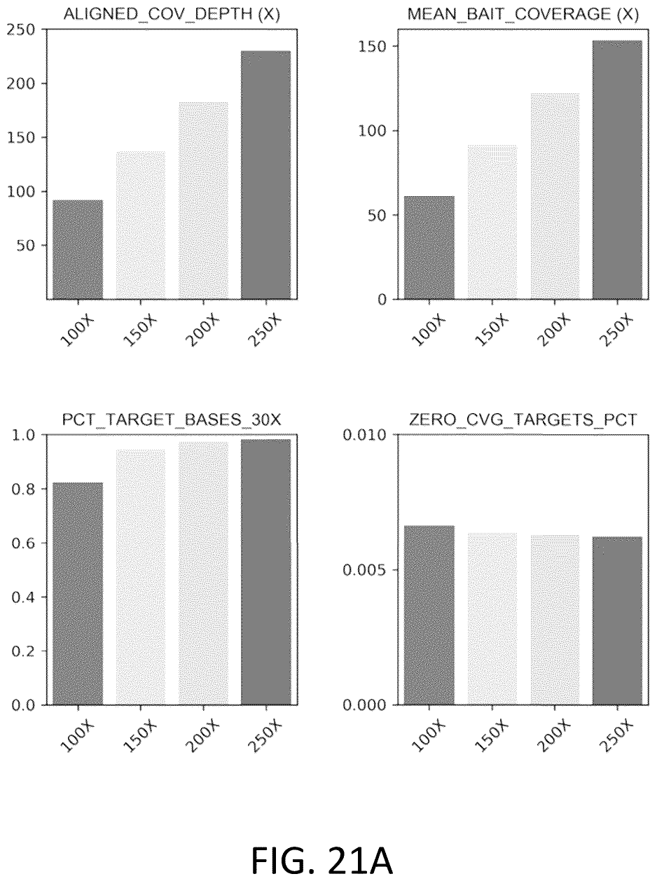

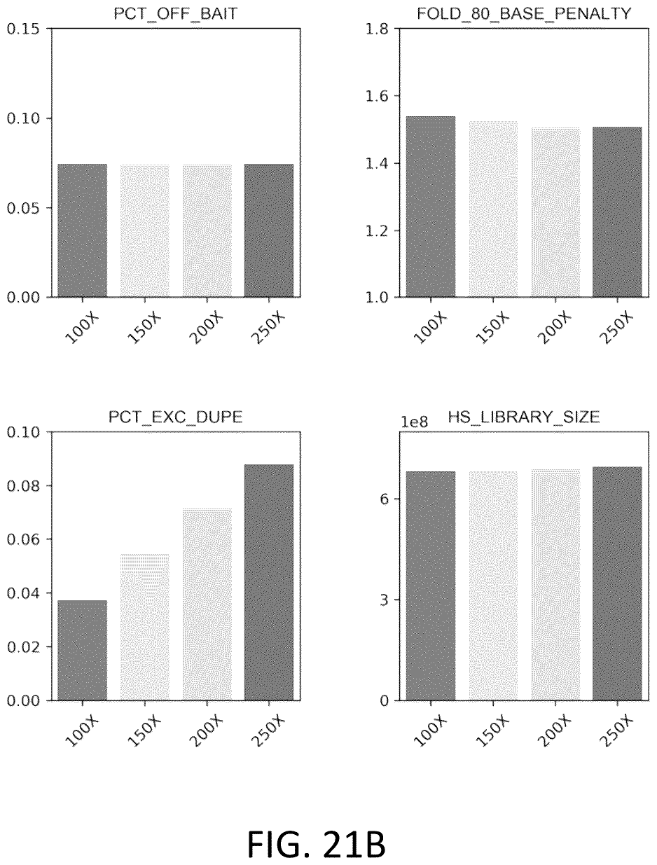

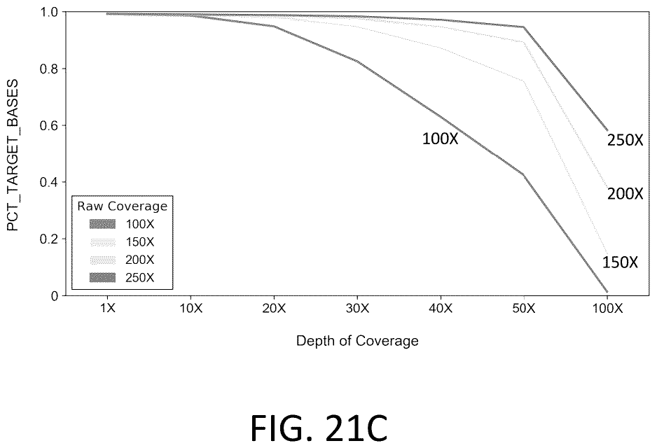

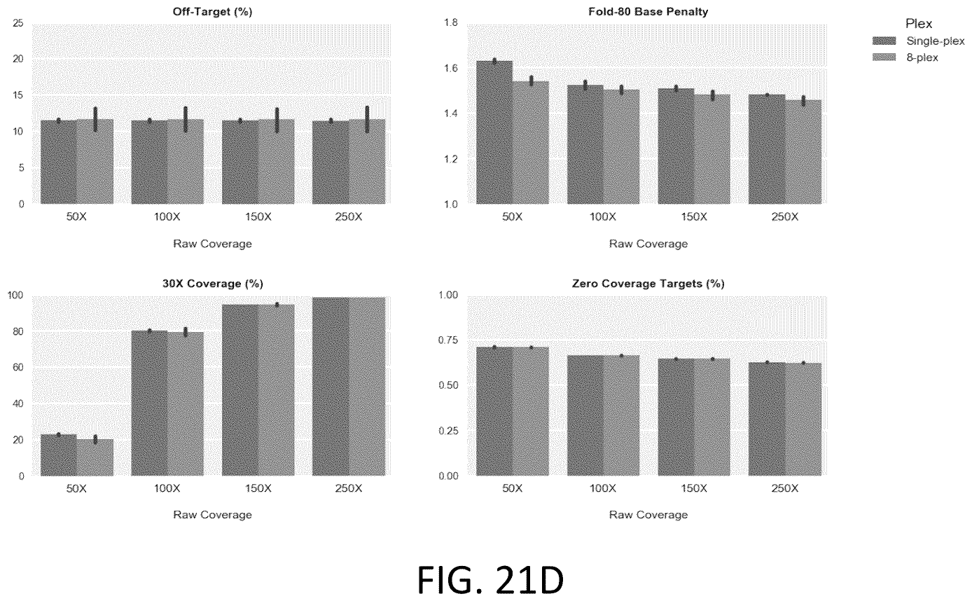

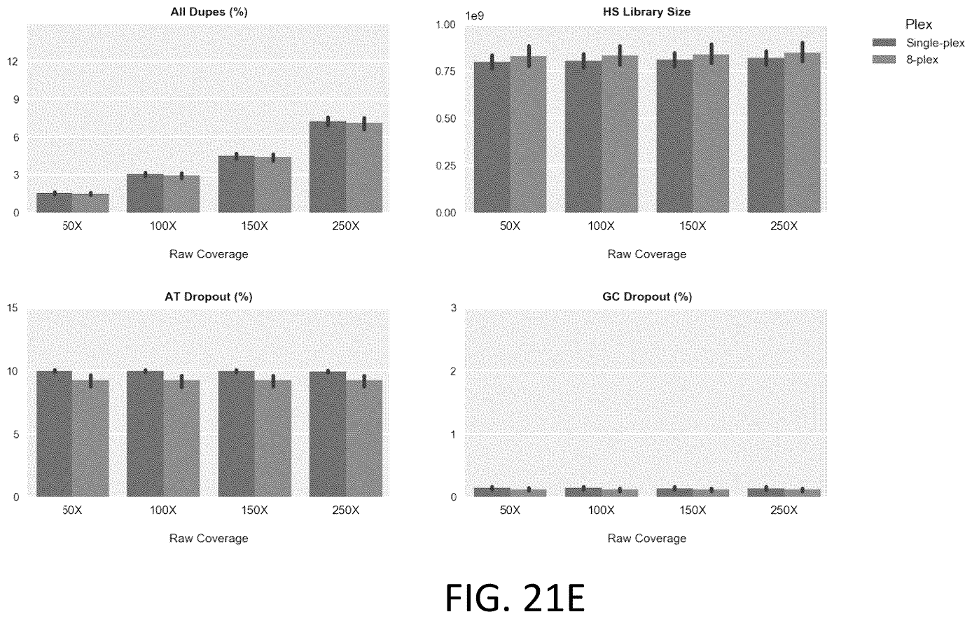

Following the general procedures of Example 11, a 123 Mb methylome targeting library was designed to cover 3.97 million CpG sites in the human genome. Targets were identified from publicly available databases such as UCSC, Ensembl, ENCODE, and others. The library comprised probes to target CpG shelves (8%), CpG shores (21%), CpG islands (15%), and CpG open seas (interCGI, 57%) as shown in FIG. 20A. Covered targets were annotated by genomic features, including: enhancers (fantom, 8,459,540), gene promoters (54,385,728), 1 to 5 kb genes (49,252,541), gene introns (90,059,139), gene exons (51,290,394), 5′UTRs (21,743,694), and 3′UTRs (10,810,132), FIG. 20B. Each feature had the total number of base pairs covered in the methylome (targets were allowed to be in more than one category to account for different transcripts). During the workflow, genomic inserts for the sample were optimized for sizes of at least 200 bases. Probe concentrations were 0.01 fmol/probe/rxn. Hybridization times were 16 hours (reducible to 4 hours), wash buffer 1 temperature was 63° C., and 2 microliters of methylation enhancer was used. Post probe capture, 10 cycles of PCR were run to amplify the genomic library. BWA-meth was used for alignments, which took about 2 hrs per sample. Single plex results after sequencing on a non-patterned flow cell of a NextSeq 550 instrument are shown in FIGS. 21A-21C. The library was also evaluated using single plex (8 cycles of post-capture PCR) and 8-plex (6 cycles of post-capture PCR) formats using a patterned flow cell of a Novaseq instrument (FIGS. 21D-21E).

### Example 18. Comparison to Commercial Methylome Panel

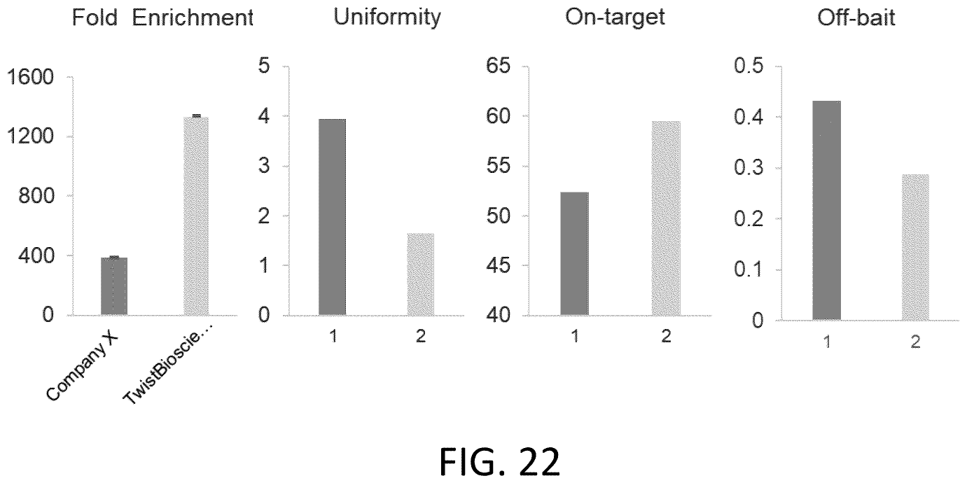

Following the general procedures of Example 11, a targeted methylation panel was prepared evaluated against a commercially available comparator panel. The targeted panel resulted in 3× better fold-performance, better uniformity, and less off-bait rate while recovering 8% more on target region reads (FIG. 22).

### Example 19. Targeted Tumor Panel

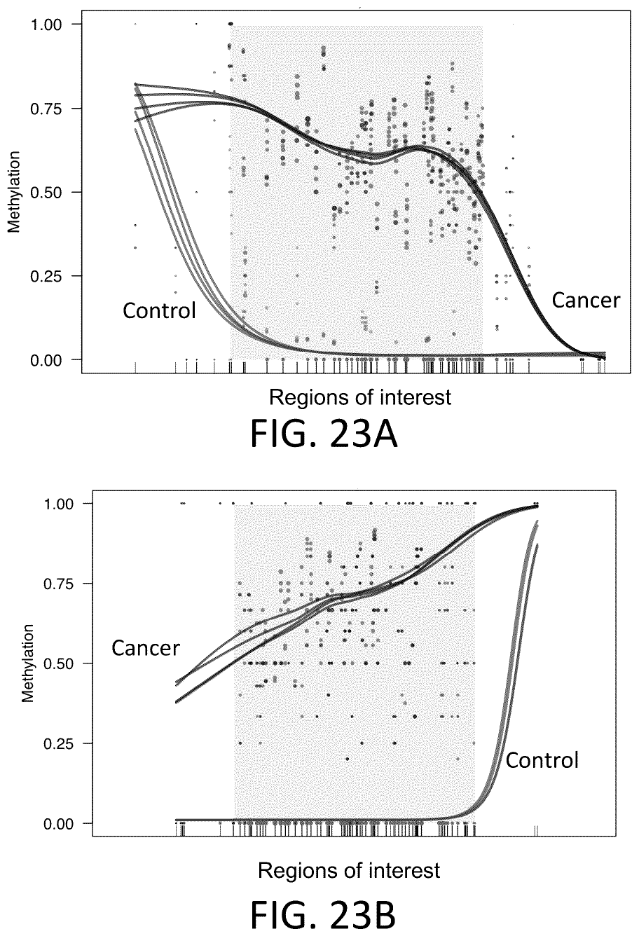

Following the general procedures of Example 11, a targeted methylation panel was prepared to target tumor signals in cfDNA. Clear differences were detected in DMRs in tumor vs. normal samples (FIGS. 23A and 23B).

### Example 20. Design and Use of Blockers for Wheat Genome

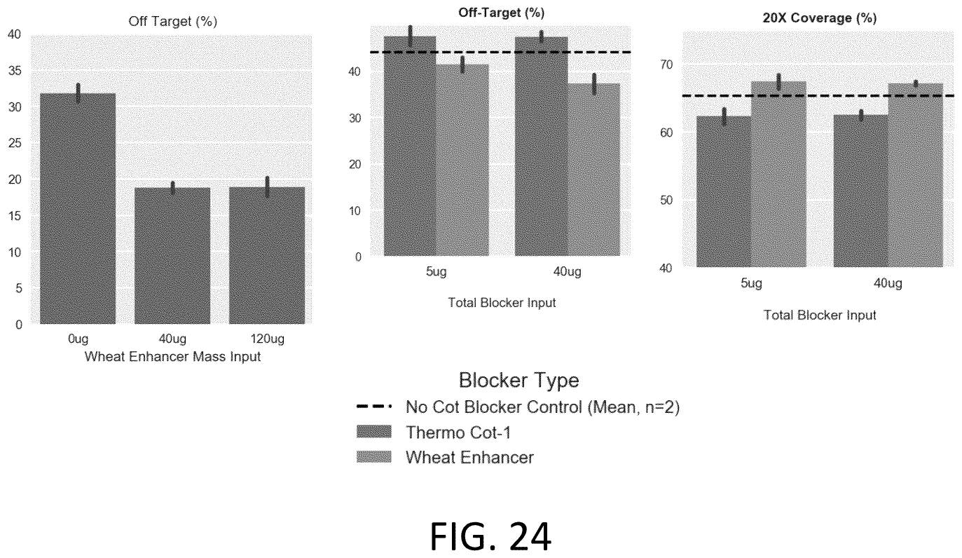

Design of synthetic blocking libraries has general applicability of designs disclosed herein to other species genomes (with or without analyzing methylation patterns). Some of the most complex and repetitive genomes such have high numbers of repeats, duplications. Wheat for example, is polyploid (hexaploidy). Following the general procedures of Example 9, a non-methylated blocker library was designed to target repetitive regions in various strains of wheat. Use of this synthetic blocker library resulted in improvement to sequencing metrics. (FIG. 24).

### Example 21. Coverage Analysis of 123 Mb Methylome Panel

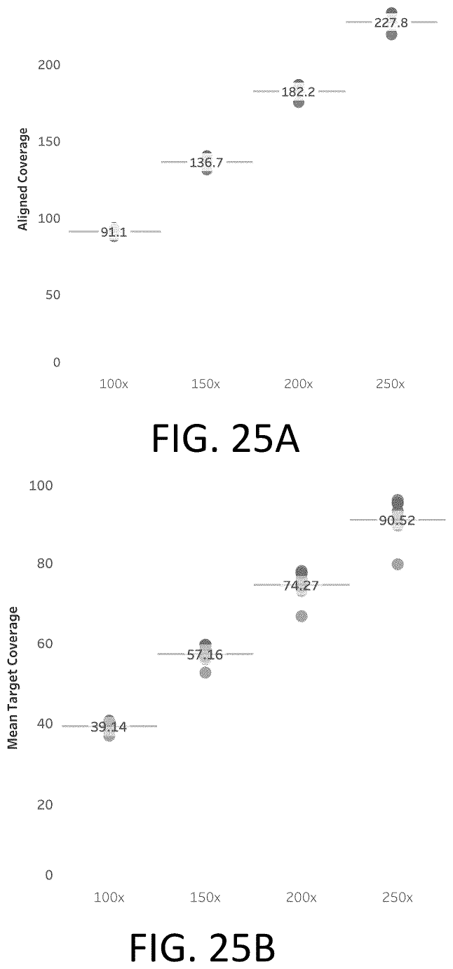

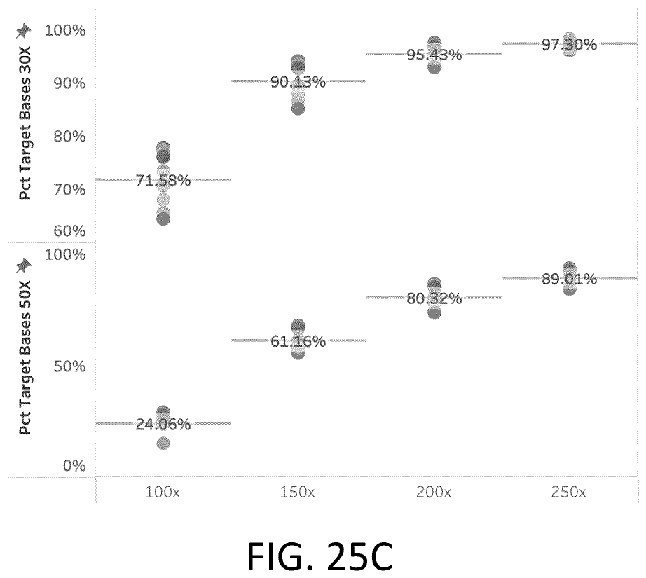

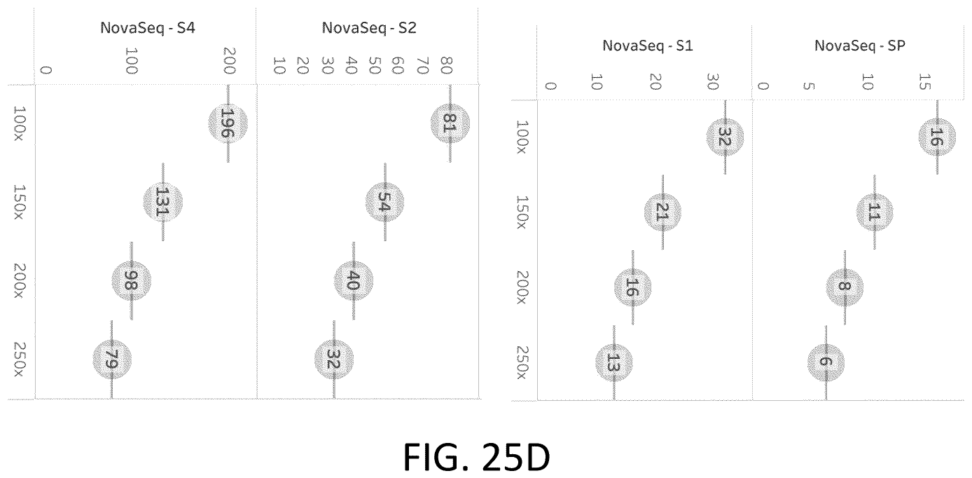

The general methods of Example 17 were used to analyze eight samples at various sequencing coverage levels (100, 150, 200, and 250×). Initial results are shown in Table 9 and FIGS. 25A-25B. Data was generated using a NovaSeq instrument in two different formats: (1) NovaSeq 2×100, 8 plex methylome experiment: 250× raw fastq files; BAM files generated by bwa-meth alignment; Picard HS metrics; CpG calls generated by MethylDackel; and Global methylation report; and (2) NovaSeq 2×100, 1 plex methylome experiment, two replicates: 250× raw fastq files; BAM files generated by bwa-meth alignment; Picard HS metrics; CpG calls generated by MethylDackel; and Global methylation report.

TABLE 9

| RAW_{—} | TOTAL_{—} | PF_BASES_{—} | ALIGNED_{—} | MEAN_BAIT_{—} | MEAN_TARGET_{—} |
|---|---|---|---|---|---|
| COVERAGE | READS | ALIGNED | COVERAGE | COVERAGE | COVERAGE |
| 250x | 203,713,852 | 28,710,239,407 | 228x | 147 | 91 |
| 200x | 162,971,082 | 22,967,781,468 | 182x | 118 | 74 |
| 150x | 122,228,312 | 17,225,757,225 | 137x | 88 | 57 |
| 100x | 81,485,542 | 11,484,056,945 | 91x | 59 | 39 |

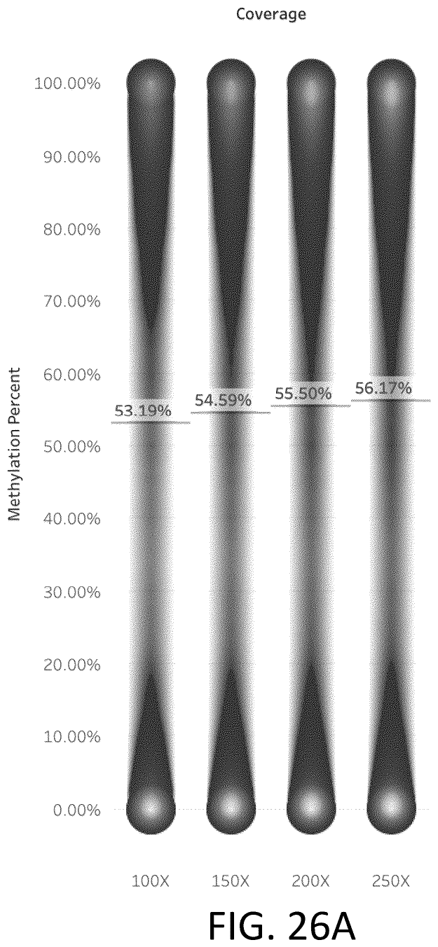

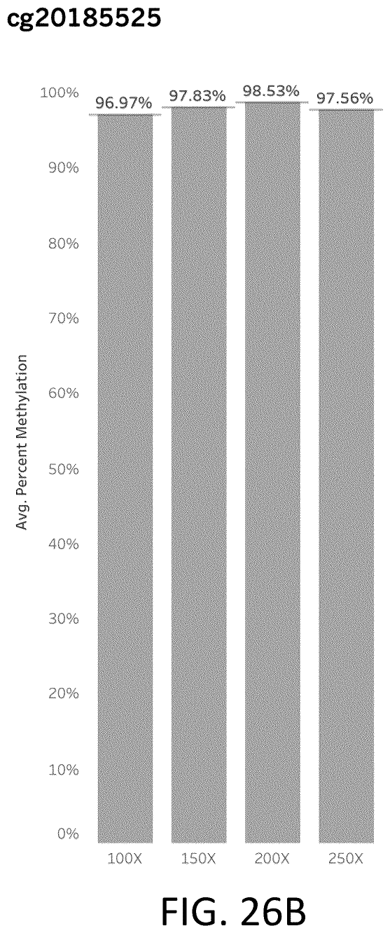

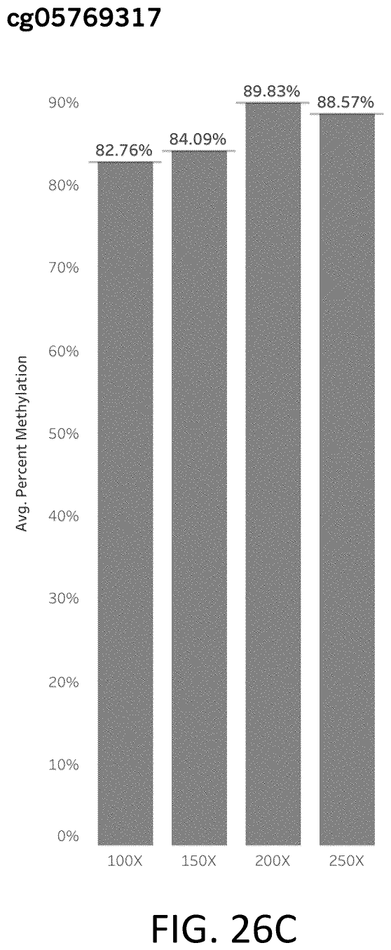

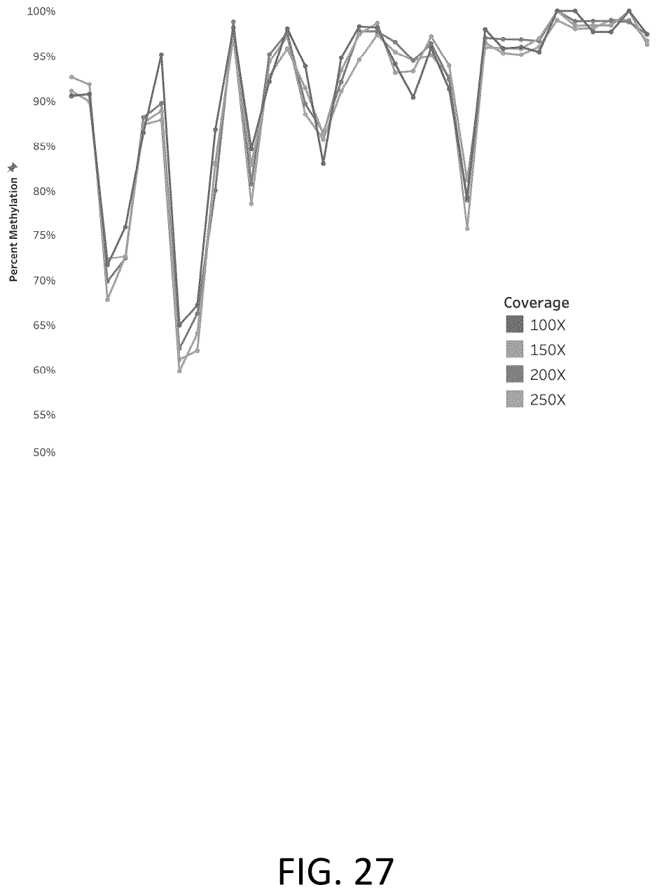

At 150× raw coverage the yield was >90% target bases above 30× usable coverage (FIG. 25C). Based on run of eight samples on a NovaSeq 2×150 run, 98 samples were multiplexed on an S4 flow cell and >200× raw coverage was achieved (FIG. 25D). The global CpG methylation percent was very similar across all four coverage depth levels (FIG. 26A-26C, including across the BRCA1 proximal enhancer region (FIG. 27).

### Example 22. Comparison of 123 Mb Methylome to Array-Based Methylome Method

Arrays in some cases are limited by background noise at the low end and signal saturation at the high end of methylation detection. A solution-based panel, such as those described herein comprise advantages over array-based methods. For example, single base resolution across the entire sequenced read for novel SNP detection is possible, along with the ability to increase sequencing coverage depth to quantify methylation fraction, and enable sense and antisense strand sequencing and detection for increased complexity.

Following the general methods of Example 21, the 123 Mb methylome panel was comparted to a commercial array-based comparator panel. Properties of the comparator panel are shown in Table 10.

TABLE 10
Design Information

| Genome | hg38 |
|---|---|
| Probe Length | 120 |

Coverage Summary

| | |
|---|---|
| Target Size (bp) | 1,728,688 |
| Total Target Regions | 859,234 |
| Target Regions with Probes | 811,260 |
| Number of probes | 670,386 |
| Number of probes removed due to repeats | 44404 |
| Non Covered Whole Target Regions (#) | 47,974 |
| Non Covered Whole Target Regions(%) | 5.56 |
| Non Covered Whole Target Regions (bp) | 96,166 |
| Covered Size (bp) | 1,631,610 |
| Overall Coverage(%) | 94.38 |

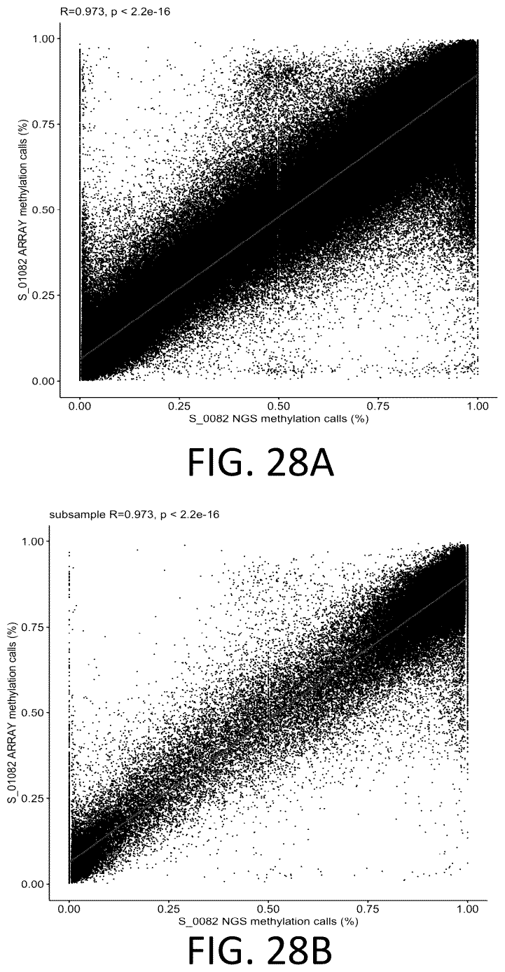

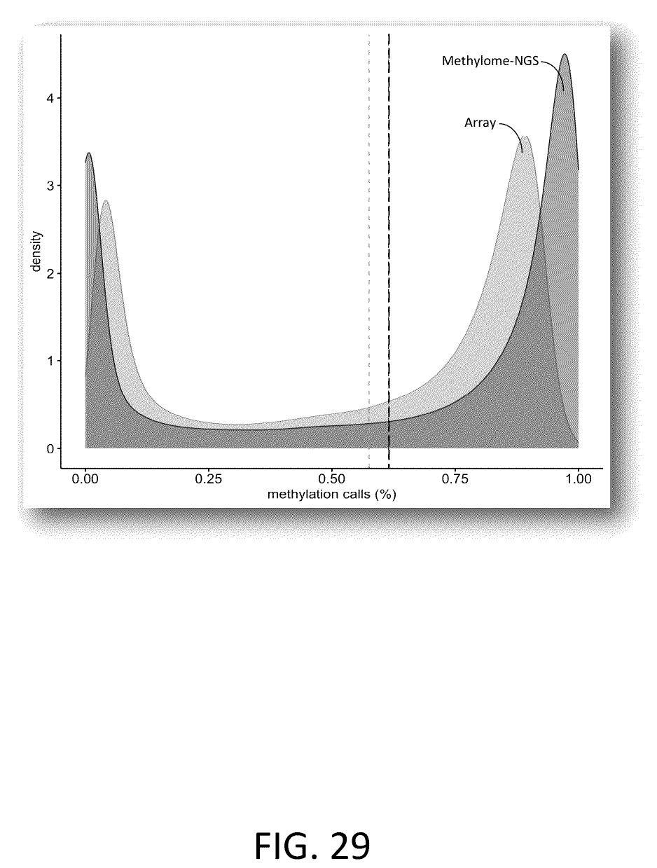

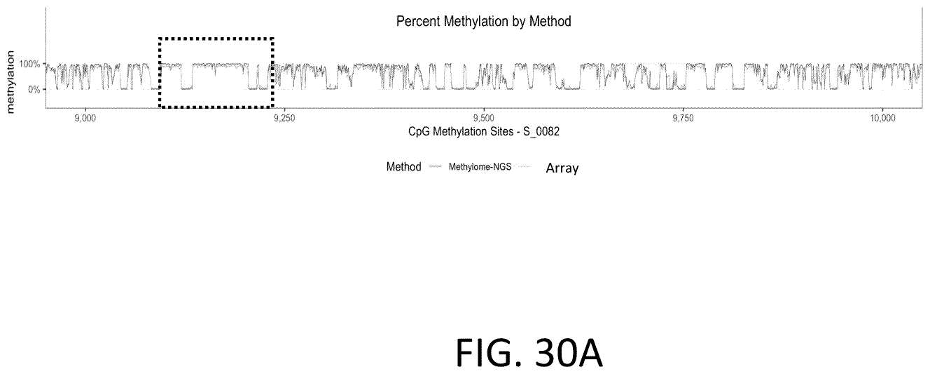

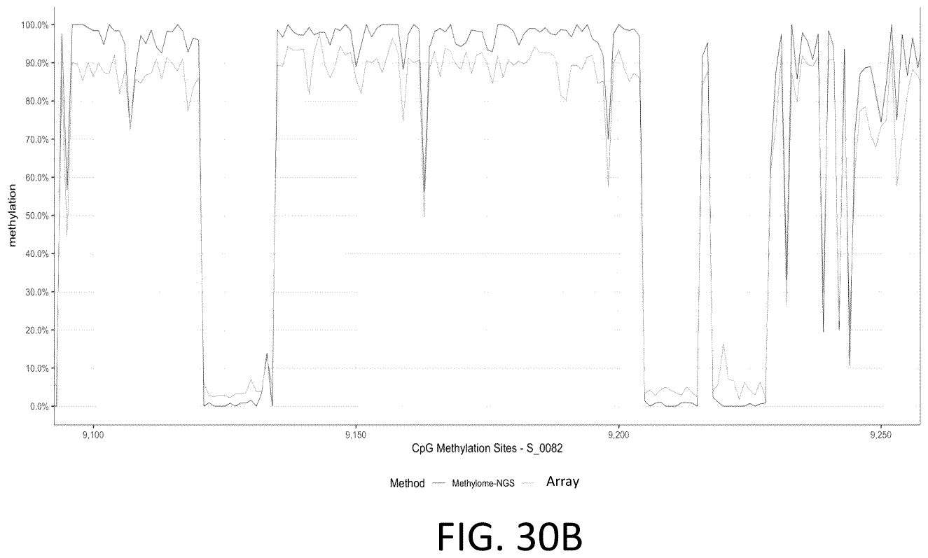

Eight samples were DNA isolated from human PBMCs. Methylation states were called for the array using estimates from the beta values. Methylation states were called for the 128 Mb panel using bwa-meth/MethylDackel (with filters min depth >10, max variant fraction of 25%, and removal of duplicates) Total overlapping CpG sites investigated were 816,485. Data sets from both the 128 Mb panel and array comparator demonstrated a high degree of correlation (FIG. 28A). Down sampling the data points to 100,000 sites more clearly showed differences in methylation percentages on the edges (where the methylome is calling 0% and 100%, FIG. 28B). The 128 Mb panel also demonstrated an increased dynamic range of methylation levels vs. the array comparator, including more accurately calling 0% and 100% methylation signals. Examining a specific genomic region, the general trends were similar for calling percent methylation states between the two methods (FIGS. 30A-30B). However, at the lower limits of methylation detection, the array had a mean delta of ~5% and at the upper limits of detection, the array had an even greater delta ~10%.

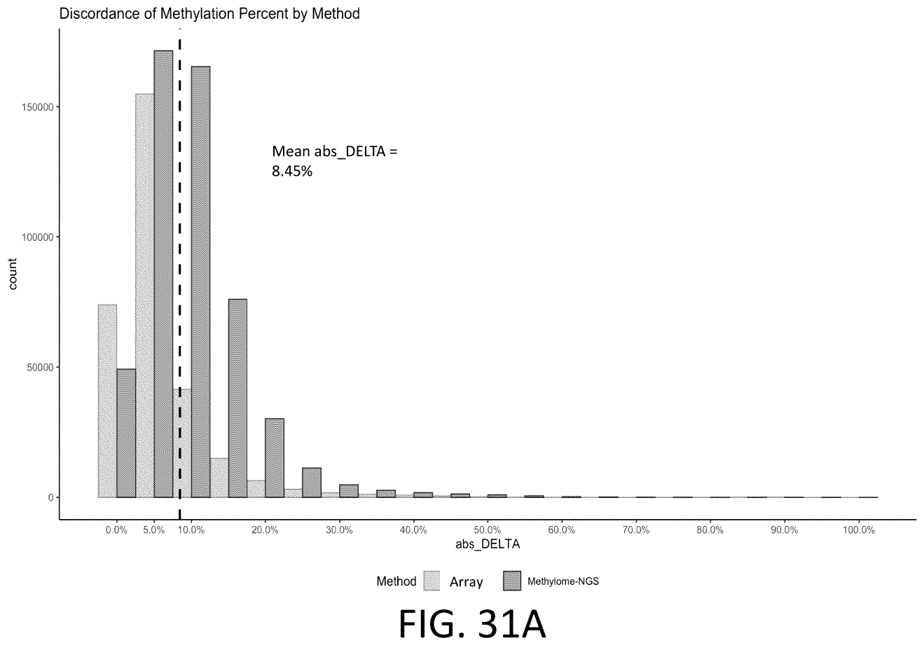

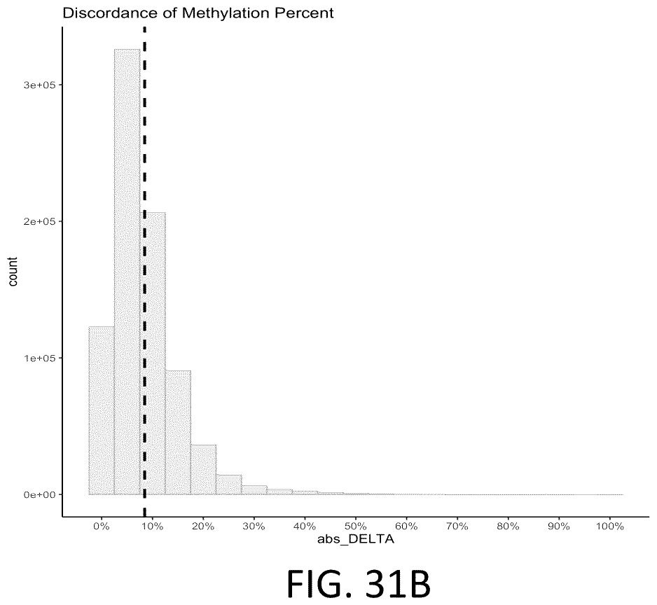

Discordance measurements indicated the array called sites at a lower % methylation than the 128 Mb panel. Bar graph bins represented which method called the higher percent methylation. In the 0-5% delta bin, the array is calling higher CpG percents. In the 5-10% delta bin, the 128 Mb panel is calling the higher CpG percents. The mean delta of percent methylation calls between the two platforms is 8.45% methylation (FIGS. 31A-31B). Of the total sites investigated (816,485): 30.0% of sites were >10% different=245,040 CpGs; 12.9% of sites were >15% different=105,281 CpGs; 5.7% of sites were >20% different=46,806 CpGs; 1.8% of sites were >30% different=14,516 CpGs; 0.3% of sites were >50% different=2,498 CpGs; and 0.02% of sites were >90% different=141 CpGs.

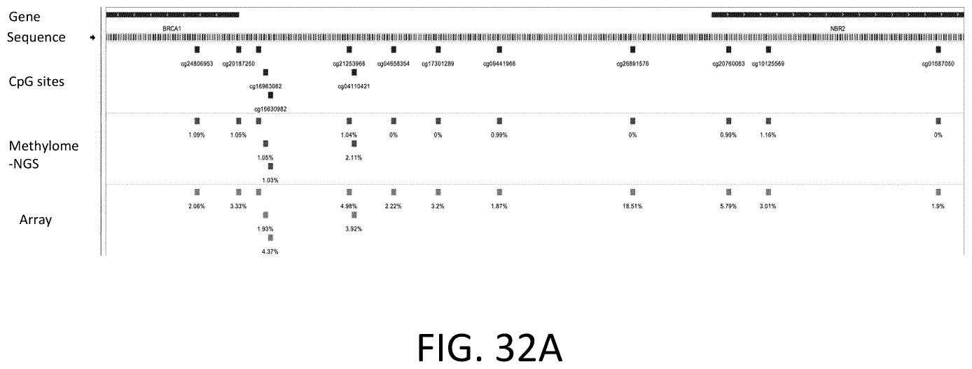

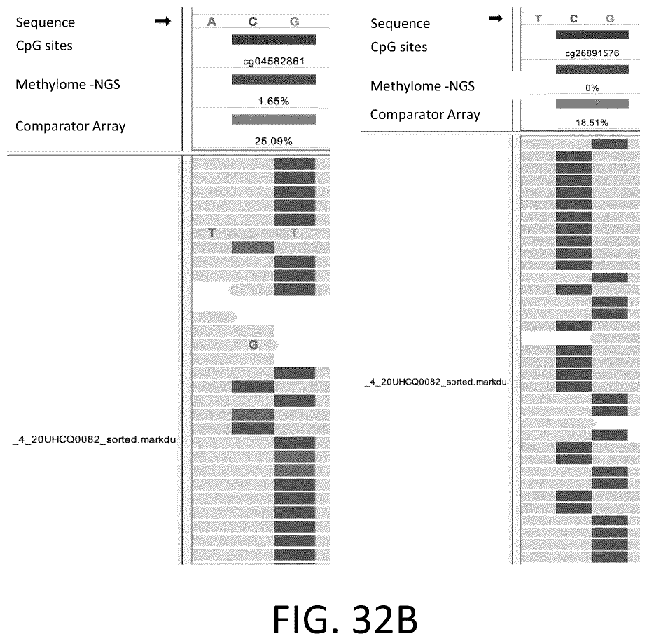

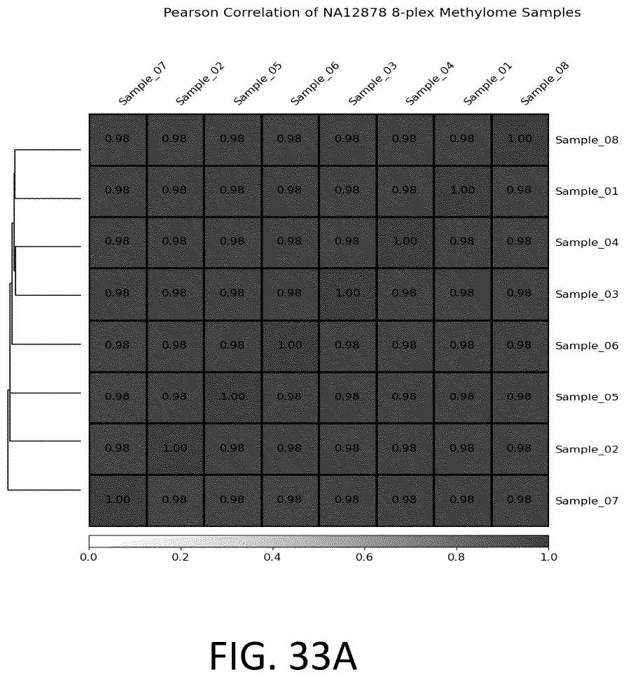

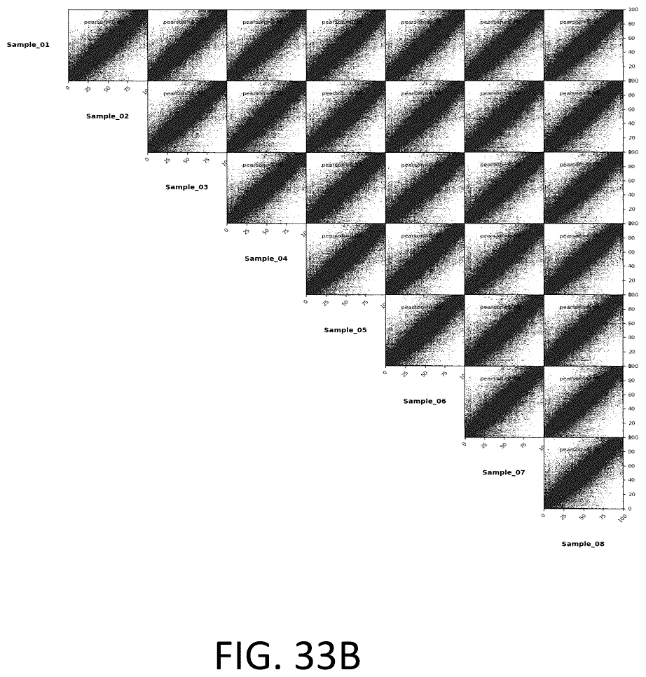

For the BRCA1 promoter region, increased dynamic range of methylation calls from the 128 Mb library method is shown clearly shown across the BRCA1 promoter region: ENCODE Accession: EH38E1864119 (chr17:43125311-43125660, FIG. 32A). Per site methylation trends were also examined (FIG. 32B). Max variant frequency of sites were filtered because they are likely variant sites (C->T mutations) instead of reading the C->T as an unmethylated site. To determine if a site was likely a variant verses an unmethylated C, MethylDackel looked at reads from the opposite strand where an A would be present if it was a C->T mutation and a G if it is C->T unmethylated conversion event. If the number of non-G's on the opposite strand for that particular site>25% (max variant fraction), then that site is excluded from the analysis. Replicates from eight NA12878 samples resulted in highly reproducible methylation calls (FIGS. 33A-33B).

### Example 23. 123 Mb Methylome Detection System

The general methods of Example 17 were used to analyze methylated samples in a methylome detection system. DNA methylation at CpG nucleotide sites in eukaryotes is a key epigenetic mark that helps regulate gene expression. Specific changes in CpG methylation occur in many human cancers, making them a promising biomarker for early cancer detection. However, existing assays can be costly, lack specificity to regions of interest and often provide only semi-quantitative estimates of the methylation fraction. This example depicts a method to address these challenges including the use of a targeted methylome panel to decrease costs associated with next generation sequencing (NGS), methylation controls to calibrate quantitative assays and UMIs for accurate deduplication in low-diversity samples.

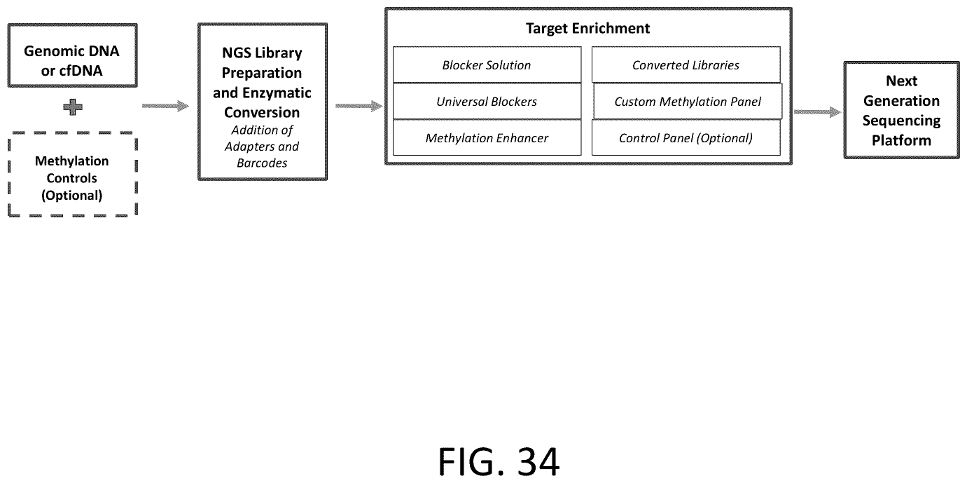

Methylation detection system. The methylation detection system consisted of (1) library preparation with enzymatic conversion and (2) target enrichment with a methylation panel (e.g., 123 Mb panel from Twist Bioscience) (FIG. 34). Conventional library preparation protocols convert unmethylated cytosines to uracils using bisulfite, which causes unwanted DNA breaks that may in some instances complicate downstream sample preparation and methylation detection. EM-seq enzymatic methylation kit (NEB) kit was used with the detection system, which led to a superior result compared to bisulfite treatment under the conditions tested.

Experimental Procedures. Genomic DNA (gDNA) was prepared for sequencing using the Methylation Detection System consisting of enzymatic methyl conversion library generation and hybrid capture using the 123 Mb panel of Example 17. CpG methylation specific controls were spiked into gDNA with different methylation rates prior to library preparation and taken through the Methylation Detection System using homologous panels to demonstrate how these controls can be used to calibrate detection assays. Additionally, libraries were generated using cell free DNA (cfDNA) and either conventional or UMI-containing adapters during the library preparation ligation step to investigate the impact on quantitative detection and total unique coverage.

Compared to chemical bisulfite conversion, enzymatic conversion with the NEBNext EM-seq kit results in high-quality DNA libraries with improved yields and longer insert sizes, each of which is crucial for maximizing sequencing and mapping efficiency. Further, bisulfite treatment in some instances is harsh to GC-rich DNA targets since conversion takes place at unmethylated cytosines. This ultimately may result in reduced coverage at high-GC target regions that are of interest in methylation sequencing applications. The approach taken by enzymatic conversion resulted in more uniform coverage across targets of varying GC content without sacrificing methylation detection sensitivity (FIG. 12B). Target enrichment can occur before or after library conversion. While post-capture conversion simplifies probe design, this approach generally requires a larger amount of DNA input since PCR amplification does not preserve DNA methylation and cannot take place before conversion. Therefore, pre-capture conversion is often the preferred approach, especially for low-input applications of methylation sequencing, such as cell-free DNA (cfDNA).

Methylome panel. The Methylome Panel (Twist Bioscience) targeted 3.98M CpG sites across 123 Mb of genomic content to target biologically relevant methylation markers. The panel targets several types of differentially methylated regions (DMR) including CpG islands, shores, shelves and open seas (FIG. 20A). Throughout all DMR, the Methylome Panel covers 84% of CpG island bases found within the human genome. 54 Mb of target bases overlap five types of Ensembl regulatory features most critical for methylation, including CTCF binding sites, enhancers, open chromatin regions, promoters and transcription factor binding sites. In addition to Ensembl, DMR and regulatory features, the design takes into consideration data found in the Genetic Testing Registry (GTR) and genes found within PubMed. In the case of PubMed, 99.9% of regions that have been cited at least 150 times are included in the panel design.

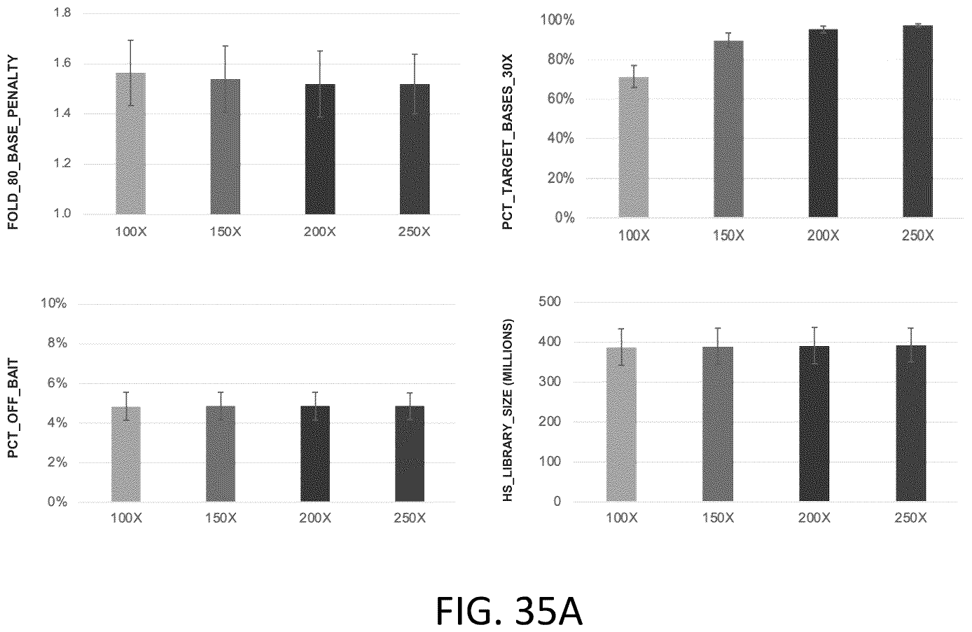

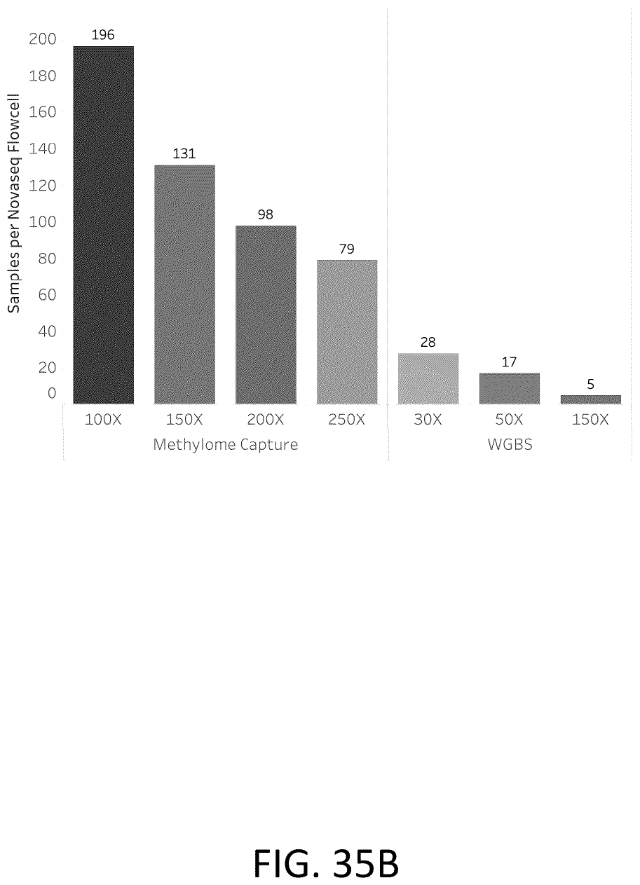

The Methylome Panel, in combination with the Methylation Detection System showed improved performance. Picard metrics demonstrated high coverage across the genome at between 100-250× downsampling, with Fold-80 Base Penalty, Percent Target Bases at 30×, Percent Off-Bait, and HS Library Size all reaching gold standard values (FIG. 35A). Downsampling to 150× coverage, 6.59 million CpG sites were detected at a minimum depth of 10×. Comparing the sequencing necessary for capture with traditional Whole Genome Bisulfite sequencing (WGBS) makes the methylome panel economic over other assays. (FIG. 35B).

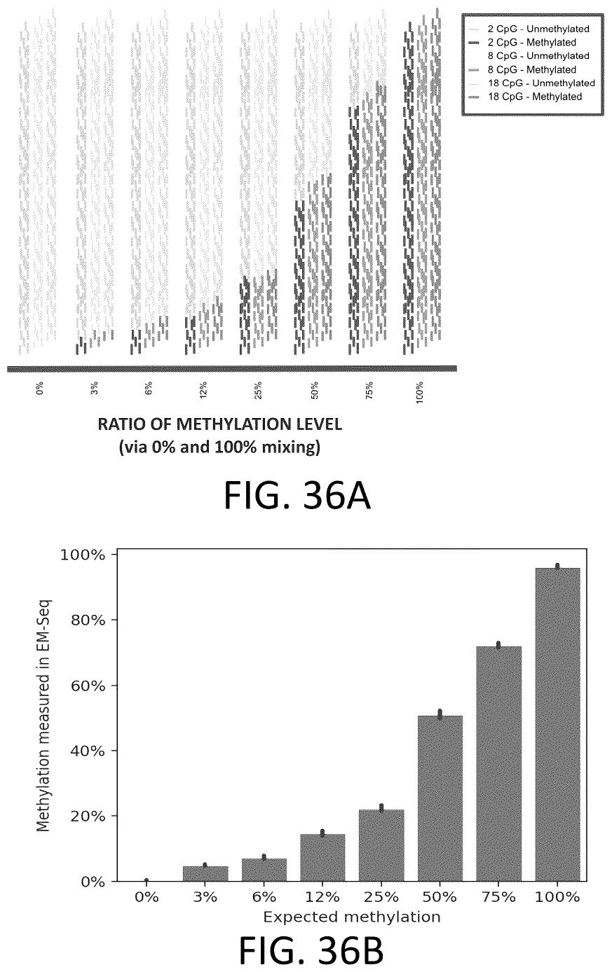

CpG Methylation Specific Controls. Methylation sequencing experiments in some instances are challenging, as experimental variation in conversion can confound the results and produce false positives. By using an inline methylation control these confounding effects can be detected, resulting in a method for calibrating methylation levels and identifying patterns using methylation target enrichment. Methylation controls (Twist Bioscience, FIG. 36A) were composed of 48 different sequences with no homology in the Refseq database and are similar in length to cfDNA (~167 bp) to be compatible with liquid biopsy workflows. Each sequence was designed to have a GC content similar to the human genome and to avoid stretches of homopolymers. These controls can be spiked directly into gDNA or cfDNA and taken through library preparation and target enrichment with the addition of a panel complementary to the controls. Measured and expected percent methylation levels were compared to determine the success of the conversion process (FIG. 36B).

Unique Molecular Identifiers in Methylation Capture. Liquid biopsy has become an increasingly important tool in the early detection of cancer and cancer-associated DNA methylation changes are promising for the development of novel biomarker tests. Using cfDNA as a template for methylation detection can pose additional challenges due to the low abundance of available material and the structure of cfDNA itself. The majority of cfDNA fragments are distributed around 167 bp in length corresponding to the length of DNA wrapped around a nucleosome. This fragmentation structure can lead to a decrease in stop-start diversity of molecules and increases the probability of calling unique molecules as duplicates. Using the Methylation Detection system may overcome these challenges as using enzymatic conversion preserves library fragment length and performing capture post conversion allows for low mass input library preparation. Additionally, methylated unique molecular identifiers (UMIs) can be added to the system to improve deduplication in low diversity insert size samples such as cfDNA. Methylated UMI adapters (e.g., Twist Bioscience) are designed to be compatible with the EM-seq library preparation protocol, target enrichment, and sequencing.

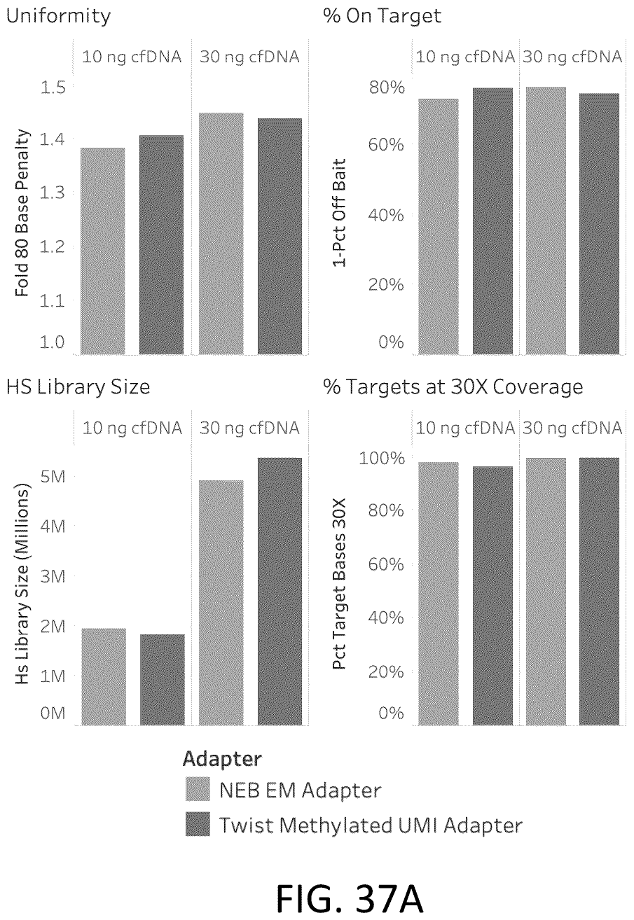

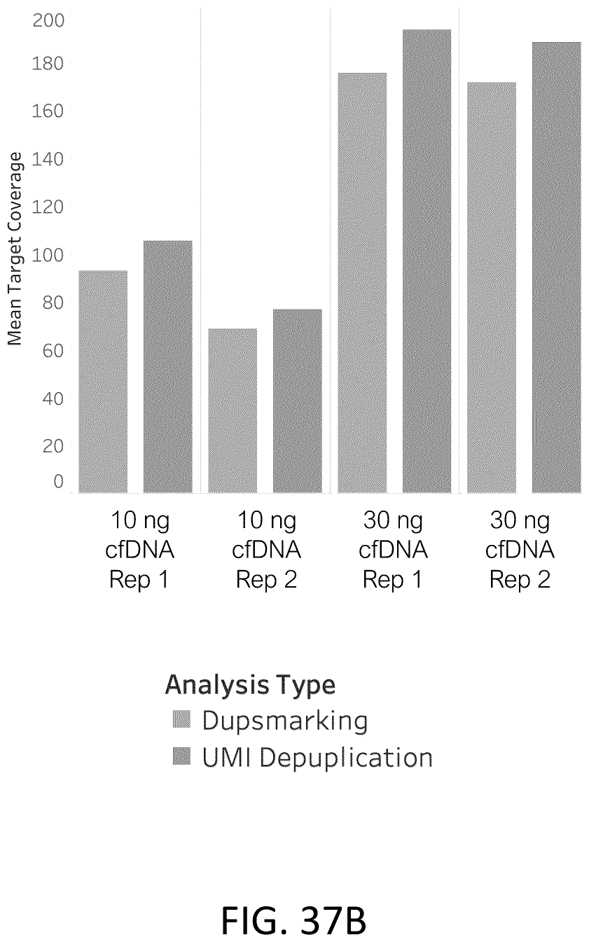

Here, cfDNA enzymatically converted libraries were captured using the Alliance Pan-Cancer Methylation panel −1.5 MB (Twist Bioscience) which covers 126K CpG sites and contains targets relevant to 31 different cancers. This workflow produced optimal Picard metrics when tested with standard methylated adapters and the Methylated UMI adapters (FIG. 37A). When using an analysis pipeline that integrates UMI information into alignments to deduplicate data, UMI informed deduplication provided higher mean target coverage over standard dupsmarking alone (FIG. 37B).

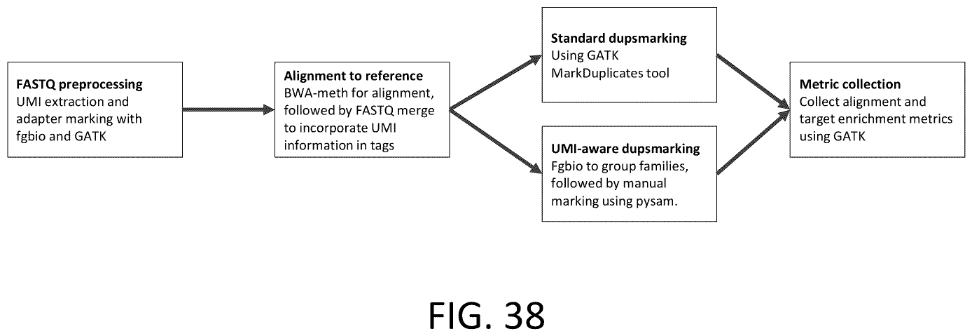

Analysis Workflow. The analysis workflow comprised a series of steps to align reads to a converted genome while preserving UMI information (FIG. 38). First, FASTQ files were pre-processed to extract UMI information, followed by alignment to a converted version of hg38 using bwa-meth. Sequencing the Methylome Panel to 100-250× raw coverage achieved uniform coverage with low off-bait Picard metrics. 6.59 million CpG sites were detected at a minimum depth of 10×. Methylome target capture allowed for informative CpG calling of up to 196 samples on a single Illumina Novaseq S4 flowcell. In contrast, performing traditional whole genome bisulfite sequencing (WGBS) without target enrichment, only 28 samples could be run on the same flowcell. CpG methylation specific controls are constructed of 48 unique contrived sequences that contain a total of 8 different levels of methylation, ranging from 100% to 0%. Including these controls allows for quantitation of methylation levels in the experimental samples and qualification of the enzymatic conversion process. After alignment, duplicates were marked either based on position alone, or based on both position and UMI sequence using custom Python scripts and the pysam library. Sequencing and enrichment metrics were collected using Picard to evaluate the success of the target enrichment portion. Plotting was done with custom Python scripts.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for detection of methylated nucleic acids, comprising:
(a) contacting a probe library comprising a plurality of polynucleotide probes with a plurality of genomic fragments comprising methylated nucleic acids and at least one blocker library having a C₀t value of no more than 2;
(b) enriching at least one genomic fragment that binds to at least one polynucleotide probe in the probe library to generate at least one enriched target polynucleotide; and
(c) sequencing the at least one enriched target polynucleotide.

2. The method of claim 1, further comprising deamination of cytosine in the plurality of genomic fragments prior to step (a).

3. The method of claim 2, wherein deamination comprises treatment with bisulfite or one or more enzymes.

4. The method of claim 3, wherein the enzyme is APOBEC (“apolipoprotein B mRNA editing enzyme, catalytic polypeptide-like”).

5. The method of claim 4, wherein the one or more enzymes are APOBEC and TET2.

6. The method of claim 1, wherein the probe library is configured to hybridize to at least one genomic fragment comprising a CpG island.

7. The method of claim 1, wherein the probe library is configured to hybridize to at least one genomic fragment comprising 5-methylcytosine or 5-hydroxymethlycytosine.

8. The method of claim 1, wherein the probe library comprises at least 5000 polynucleotide probes.

9. The method of claim 1, wherein the probe library comprises at least 1 million polynucleotide probes.

10. The method of claim 1, wherein the probe library comprises at least 10 million polynucleotide probes.

11. The method of claim 1, wherein the polynucleotide probes are 80-250 bases in length.

12. The method of claim 1, wherein the polynucleotide probes are double stranded.

13. The method of claim 1, wherein the polynucleotide probes are configured to bind to both sense and antisense regions of a target polynucleotide.

14. The method of claim 1, wherein the polynucleotide probes are configured to bind to both treated and untreated genomic fragments.

15. The method of claim 1, wherein the library is present in at least 5 fold molar excess over the plurality of genomic fragments.

16. The method of claim 1, wherein the polynucleotide probes comprise at least one detectable label.

17. The method of claim 1, wherein the polynucleotide probes collectively comprise at least 1 million bases.

18. The method of claim 17, wherein the polynucleotide probes collectively comprise at least 10 million bases.

19. The method of claim 17, wherein the polynucleotide probes collectively comprise at least 100 million bases.

20. The method of claim 1, wherein the polynucleotide probes collectively target one or more of CpG islands, CpG shores, CpG shelves and CpG open seas.

21. The method of claim 20, wherein the polynucleotide probes collectively target at least 3 million CpG sites.

22. The method of claim 21, wherein the polynucleotide probes collectively target at least 6 million CpG sites.

23. The method of claim 20, wherein the polynucleotide probes collectively target at least 80% of all CpG sites in the human genome.

24. The method of claim 20, wherein the polynucleotide probes collectively target at least 25 million bases comprising one or more regulatory features involved in methylation.

25. The method of claim 24, wherein the regulatory features comprise CTCF binding sites, enhancers, open chromatin regions, promoters or transcription factor binding sites.

26. The method of claim 1, wherein the polynucleotide probes are not attached to a solid support during enriching.

27. The method of claim 1, wherein binding comprises hybridization.

28. The method of claim 1, wherein at least some of the polynucleotide probes are configured to bind to BRCA1 proximal enhancer region.

29. The method of claim 1, wherein the polynucleotide probes are configured to bind to CpG sites associated with at least one cancer.

30. The method of claim 29, wherein the polynucleotide probes are configured to bind to CpG sites associated with at least 20 cancers.

31. The method of claim 1, wherein sequencing comprises sequencing by synthesis, nanopore sequencing, or SMRT sequencing.

32. The method of claim 31, wherein at least 5 million CpG sites are sequenced with at least 10× coverage.

33. The method of claim 1, wherein the method further comprises contacting the library with salmon sperm in step (a).

34. The method of claim 1, wherein contacting occurs for no more than 4 hours.

35. The method of claim 1, wherein contacting occurs at a temperature of 60-70 degrees C.

36. The method of claim 1, wherein at least some of genomic fragments comprise at least one polynucleotide adapter.

37. The method of claim 36, wherein the at least one polynucleotide adapter comprises at least one index sequence.

38. The method of claim 37, wherein the at least one index sequence is 8-16 bases in length.

39. The method of claim 1, wherein the method further comprises contacting the library with one or more universal blockers in step (a).

40. The method of claim 1, wherein the plurality of genomic fragments comprise cell-free DNA.

41. The method of claim 1, wherein the plurality of genomic fragments comprise ctDNA.

42. The method of claim 1, wherein the plurality of genomic fragments are 100-200 bases in length.

43. The method of claim 1, wherein the plurality of genomic fragments each comprise at least one adapter.

44. The method of claim 43, wherein the adapter comprises a unique molecular identifier (UMI).

45. The method of claim 44, wherein the adapter comprises at least one methylated base.

46. The method of claim 45, wherein all cytosines in the adapter are methylated.

47. A methylome detection system comprising:
(a) the probe library of any one of claims 1-46;
(b) one or more enzymes configured to distinguish methylated from unmethylated bases;
(c) a plurality of sample nucleic acids; and
(d) optionally a methylation control.

48. The system of claim 47, wherein the methylation control comprises a plurality of polynucleotides comprising at least eight different amounts of methylation.

49. The system of claim 47, wherein the methylation control comprises at least 24 different sequences.

50. The system of claim 47, wherein the methylation control comprises at least 24 different sequences having no homology to sequences in the Refseq database.

51. The system of claim 47, wherein the methylation control comprises polynucleotides having an average length of 150-175 bases.

52. The system of claim 47, wherein the methylation control comprises polynucleotides having an average length of about 167 bases.

53. The system of claim 47, wherein the sample nucleic acids comprise cell-free DNA.

54. The system of claim 53, wherein the plurality of genomic fragments comprise ctDNA.
